(19) United States
(12) Patent Application Publication
(10) Pub. No.: US 2023/0295292 A1
(43) Pub. Date: Sep. 21, 2023

(54) METHODS OF TREATING CANCERS AND ENHANCING EFFICACY OF GPRC5DXCD3 BISPECIFIC ANTIBODIES

(71) Applicant: Janssen Biotech, Inc., HORSHAM, PA (US)

(72) Inventors: Jenna Goldberg, Raritan, NJ (US); Shun Xin Wang Lin, Spring House, PA (US); Thomas J. Prior, Spring House, PA (US); Raluca Verona, Spring House, PA (US); Brendan Weiss, Spring House, PA (US)

(21) Appl. No.: 18/052,172

(22) Filed: Nov. 2, 2022

Related U.S. Application Data
(60) Provisional application No. 63/275,356, filed on Nov. 3, 2021.

(51) Int. Cl. C07K 16/28; A61P 35/00; A61K 31/454; A61K 31/573; A61K 39/395

(52) U.S. Cl. CPC C07K 16/28; A61K 31/454; A61K 31/573; A61K 39/3955; A61P 35/00; C07K 16/2809; C07K 16/2896; A61K 2039/545; C07K 2317/31

(57) ABSTRACT

Disclosed are methods of treating cancers and enhancing efficacy of T cell redirecting therapeutics. In particular, methods are disclosed of using a GPRC5DxCD3 bispecific antibody, an anti-CD38 antibody and/or pomalidomide to treat cancers.

### CROSS-REFERENCE TO RELATED APPLICATIONS

This application claims the benefit of U.S. Provisional Application Ser. No. 63/275.356 filed 03 November, 2021, the entire content of which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Nov. 1, 2022, is named “258199.060801_PRD4189USNP1_SL.xml” and is 64.2 kilobytes in size.

### FIELD OF THE INVENTION

Disclosed are methods of treating cancers and enhancing efficacy of GPRC5DxCD3 bispecific antibodies.

### BACKGROUND OF THE INVENTION

From 1990-2016 Multiple Myeloma (MM) incident cases increased by 126%, and deaths increased by 94% worldwide. In the United States in 2020. it is estimated that there will be 32,270 newly diagnosed cases of MM with 140,779 people living with the disease. In Europe, multiple myeloma is the second most common hematologic malignancy, with 35,842 new cases estimated in the 27 European countries in 2020 (European Cancer Information System 2020). Despite multiple therapeutic options, the disease most often recurs and remains incurable. With each successive relapse, symptoms return, quality of life worsens, and the chance and duration of response typically decreases.

Relapsed and refractory multiple myeloma constitutes a specific unmet medical need. Patients who progress after receiving standard therapies (such as proteasome inhibitors [PI] or immunomodulatory drugs [IMiDs]) are challenging to treat since they have already been exposed to 2 major drug classes, and new effective and convenient treatment options are needed. Despite myeloma remaining an incurable disease, <40% of patients receive a third line of therapy. Lenalidomide-containing regimens have increasingly become standard-of-care first-line therapies in the US and in most of Europe both in transplant-eligible and transplant-ineligible patients. As a result of these practice patterns, most patients with relapsed multiple myeloma are lenalidomide-exposed when they first relapse.

T cell redirected killing is a desirable mode of action in many therapeutic areas. In general T cell redirecting molecules are engineered to have at least two antigen binding sites wherein one site binds a surface antigen on a target cell and the other site binds a T cell surface antigen. Amongst T cell surface antigens, the human CD3 epsilon subunit from the TCR protein complex has been the most targeted to redirect T cell killing. Various bispecific antibody formats have been shown to mediate T cell redirection in both in pre-clinical and clinical investigations.

Tumors evade immune recognition through creating an immunosuppressive tumor microenvironment (TME). In the TME, under conditions of persistent antigen and inflammation, T cells become exhausted, or dysfunctional, and progressively lose their effector function and proliferative capacity. Impaired function and number of available T cells to engage therapeutics mediating T cell redirected killing may impair anti-tumor efficacy of the therapeutic. There is a need to enhance T cell functionality for optimal efficacy of the therapeutics mediating T cell redirected killing.

Talquetamab (Tal) is a bispecific antibody that binds to G Protein-Coupled Receptor Class C Group 5 Member D (GPRC5D) and CD3. Daratumumab (Dara) is a monoclonal antibody approved for MM treatment that targets CD38 on MM cells, resulting in direct cytotoxicity of MM cells. Novel agents are needed for treating cancer. particularly multiple myeloma (MM), which remains incurable with most patients (pts) relapsing or becoming refractory to standard therapies.

### SUMMARY OF THE INVENTION

It is now discovered that, using a dosage regimen described in this application, a combination of a GPRC5DxCD3 bispecific antibody and an anti-CD38 antibody DARZALEX® (daratumumab), each of which mediates killing of multiple myeloma cells upon target engagement on the same cell, can be safely administered to a subject to treat a cancer, particularly a relapsed/refractory MM.

In one general aspect, the application relates to a method of treating a cancer, such as multiple myeloma, in a subject in need thereof, comprising:
(1) administering to the subject a GPRC5DxCD3 bispecific antibody at a dose of 60 µg/kg to 1200 µg/kg every 1-2 weeks, and
(2) subcutaneously administering to the subject an anti-CD38 antibody at a dose of 1200 mg to 2400 mg every 1-4 weeks.

In some embodiment, the GPRC5DxCD3 bispecific antibody is administered to the subject at a dose of 60 µg/kg to 1200 µg/kg, such as 60 µg/kg, 70 µg/kg, 80 µg/kg, 90 µg/kg, 100 µg/kg, 200 µg/kg, 250 µg/kg, 300 µg/kg, 350 µg/kg, 400 µg/kg, 450 µg/kg, 500 µg/kg, 550 µg/kg, 600 µg/kg, 650 µg/kg, 700 µg/kg, 750 µg/kg, 800 µg/kg, 200 µg/kg, 250 µg/kg, 300 µg/kg, 400 µg/kg, 450 µg/kg, 500 µg/kg, 550 µg/kg, 600 µg/kg, 700 µg/kg, 750 µg/kg, 800 µg/kg, 850 µg/kg, 900 µg/kg, 950 µg/kg, 1000 µg/kg, 1050 µg/kg, 1100 µg/kg, 1150 µg/kg, or 1200 µg/kg, or any dose in-between every 1-2 weeks.

In some embodiments, the method comprises:
(1) subcutaneously administering to the subject the GPRC5DxCD3 bispecific antibody at a dose of 300 µg/kg to 1200 µg/kg every 1-2 weeks, and
(2) subcutaneously administering to the subject the anti-CD38 antibody at a dose of 1600 mg to 2000 mg every 1-4 weeks.

In some embodiment, the method further comprises subcutaneously administering to the subject the GPRCSDxCD3 bispecific antibody at a dose lower than the dose used in step (1) prior to step (1).

According to embodiments of the application, the GPRC5DxCD3 bispecific antibody is subcutaneously administered to the subject at a dose of about 300 µg/kg, 400 µg/kg. 450 µg/kg, 500 µg/kg, 550 µg/kg, 600 µg/kg, 700 µg/kg, 750 µg/kg, 800 µg/kg, 850 µg/kg, 900 µg/kg, 950 µg/kg, or 1000 µg/kg or any dose in-between, once every week or once every two weeks. For example, the GPRC5DxCD3 bispecific antibody can be subcutaneously administered to the subject at a dose of 400 µg/kg weekly or biweekly, or 800 µg/kg biweekly

In an embodiment of the application, the GPRC5DxCD3 bispecific antibody is subcutaneously administered to the subject at a dose of 400 µg/kg weekly or 800 µg/kg biweekly, and the anti-CD38 antibody is subcutaneously administered to the subject in the dose of 1800 mg once every week during week 1 to week 8 of the treatment, once every two weeks during week 9 to week 24 of the treatment, and once every four weeks after week 24 of the treatment. The anti-CD38 antibody is administered or provided for administration together with rHuPH20, such as about 30,000 U of rHuPH20.

Any suitable GPRC5DxCD3 bispecific antibody can be used in a method of the application. In some embodiments, a GPRC5DxCD3 bispecific antibody useful for the application comprises:
(i) a GPRC5D binding domain comprising a heavy chain variable region (VH) having heavy chain complementarity determining regions (HCDRs) HCDR1, HCDR2 and HCDR3 of the amino acid sequences of SEQ ID NO: 27, SEQ ID NO: 28, and SEQ ID NO: 29, respectively, and a light chain variable region (VL) having light chain complementarity determining regions (LCDRs) LCDR1, LCDR2 and LCDR3 of the amino acid sequences of SEQ ID NO: 30, SEQ ID NO: 31, and SEQ ID NO: 32. respectively, and
(ii) a CD3 binding domain comprising a VH having HCDR1, HCDR2 and HCDR3 of the amino acid sequences of SEQ ID NO: 17, SEQ ID NO: 18, and SEQ ID NO: 19, respectively, and a VL having LCDR1, LCDR2 and LCDR3 of the amino acid sequences of SEQ ID NO: 20, SEQ ID NO: 21, and SEQ ID NO: 22, respectively.

In some embodiment, the GPRC5D binding domain comprises the VH having the amino acid sequence of SEQ ID NO: 33 and the VL having the amino acid sequence of SEQ ID NO: 34. The CD3 binding domain comprises the VH having the amino acid sequence of SEQ ID NO: 23 and the VL having the amino acid sequence of SEQ ID NO: 24. Preferably, the GPRC5DxCD3 bispecific antibody comprises a first heavy chain (HC1) having the amino acid sequence of SEQ ID NO: 35, a first light chain (LC1) having the amino acid sequence of SEQ ID NO: 36. a second heavy chain (HC2) having the amino acid sequence of SEQ ID NO: 25, and a second light chain (LC2) having the amino acid sequence of SEQ ID NO: 26. More preferably, the GPRC5D xCD3 bispecific antibody is talquetamab.

In some embodiments, the anti-CD38 antibody comprises a VH having HCDR1, HCDR2 and HCDR3 of the amino acid sequences of SEQ ID NO: 7, SEQ ID NO: 8, and SEQ ID NO: 9, respectively, and a VL having LCDR1, LCDR2 and LCDR3 of the amino acid sequences of SEQ ID NO: 10, SEQ ID NO: 11, and SEQ ID NO: 12, respectively. Preferably, the CD38 antibody comprises the VH having the amino acid sequence of SEQ ID NO: 5, and the VL having the amino acid sequence of SEQ ID NO: 6. More preferably, the CD38 antibody is daratumumab.

In one embodiment, the application relates to a method of treating multiple myeloma in a subject in need thereof, comprising:
(1) subcutaneously administering to the subject a weight based treatment dose of 400 µg/kg of a GPRC5DxCD3 bispecific antibody weekly: or 800 µg/kg of a GPRC5DxCD3 bispecific antibody biweekly, and
(2) subcutaneously administering to the subject 1800 mg of an anti-CD38 antibody once every week during week 1 to week 8 of the treatment, once every two weeks during week 9 to week 24 of the treatment, and once every four weeks after week 24 of the treatment,
wherein the GPRC5DxCD3 bispecific antibody comprises a first heavy chain (HC1) of SEQ ID NO: 35, a first light chain (LC1) of SEQ ID NO: 36, a second heavy chain (HC2) of SEQ ID NO: 25, and a second light chain (LC2) of SEQ ID NO: 26, and the anti-CD38 antibody comprises the HC of SEQ ID NO: 13 and the LC of SEQ ID NO: 14.

In some embodiment, the method further comprises subcutaneously administering to the subject step-up doses of 10 µg/kg and 60 µg/kg of the GPRC5DxCD3 bispecific antibody prior to the initial subcutaneous administration of the weight based treatment dose of 400 µg/kg of the GPRC5DxCD3 bispecific antibody, or the method further comprises subcutaneously administering to the subject step-up doses of 10 µg/kg, 60 µg/kg and 300 µg/kg of the GPRC5DxCD3 bispecific antibody prior to the initial subcutaneous administration of the weight based treatment dose of 800 µg/kg of the GPRC5DxCD3 bispecific antibody.

In some embodiments of the application, the subject has received at least one prior treatment of multiple myeloma, preferably, the subject is relapsed or refractory to the at least one prior treatment, more preferably, the prior treatment comprises at least one of a proteasome inhibitor (PI) and an immunomodulatory agent (IMiD). The subject can be refractory or relapsed to a treatment, such as a treatment selected from the group consisting of an anti-CD38 antibody, lenalidomide, bortezomib, pomalidomide, carfilzomib, elotuzumab, ixazomib, melphalan and thalidomide, or any combination thereof. Preferably, if the subject has received only 1 prior line of therapy, the subject is lenalidomide refractory.

In some embodiments, a method of the application further comprises administering to the subject another treatment for the cancer, such as pomalidomide and/or dexamethasone.

In some embodiments, a method according to embodiments of the application results in T-cell activation, such as an increase in at least one of CD25, PD-1, CD38 on CD4 and CD8 T cells. A method according to embodiments of the application can also result in an increase in frequency of at least one of CD38+ CD8+ T cells, CD38+ CD4+ T cells and Tregs T cells.

Another general aspect of the application relates to an anti-CD38 antibody as described herein for use in a method of treating a cancer, such as MM, more particularly a relapsed or refractory MM, wherein the method comprises:
(1) administering to the subject a GPRC5DxCD3 bispecific antibody at a dose of 60 µg/kg to 1200 µg/kg or 300 µg/kg to 1200 µg/kg every 1-2 weeks, and
(2) subcutaneously administering to the subject the anti-CD38 antibody at a dose of 1200 mg to 2400 mg every 1-4 weeks.

Another general aspect of the application relates to a GPRC5DxCD3 bispecific antibody as described herein for use in treating a cancer, such as MM, more particularly a relapsed or refractory MM, wherein the treatment comprises:
(1) administering to the subject the GPRC5DxCD3 bispecific antibody at a dose of 60 µg/kg to 1200 µg/kg or 300 µg/kg to 1200 µg/kg every 1-2 weeks, and
(2) subcutaneously administering to the subject an anti-CD38 antibody at a dose of 1200 mg to 2400 mg every 1-4 weeks.

Yet another general aspect of the application relates to a combination or a kit of an anti-CD38 antibody as described herein and a GPRC5DxCD3 bispecific antibody as described herein for use in treating a cancer, such as MM, more particularly a relapsed or refractory MM, wherein the treatment comprises:
(1) administering to the subject a GPRC5DxCD3 bispecific antibody at a dose of 60 µg/kg to 1200 µg/kg or 300 µg/kg to 1200 µg/kg every 1-2 weeks, and
(2) subcutaneously administering to the subject the anti-CD38 antibody at a dose of 1200 mg to 2400 mg every 1-4 weeks.

The application further relates to use of a combination of an anti-CD38 antibody as described herein and a GPRC5DxCD3 bispecific antibody as described herein in the manufacture of a medicament for treating a cancer, such as MM, more particularly a relapsed or refractory MM, wherein the treatment comprises:
(1) administering to the subject a GPRC5DxCD3 bispecific antibody at a dose of 60 µg/kg to 1200 µg/kg or 300 µg/kg to 1200 µg/kg every 1-2 weeks, and
(2) subcutaneously administering to the subject the anti-CD38 antibody at a dose of 1200 mg to 2400 mg every 1-4 weeks.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 is a schematic of overview of Part 1 and Part 2 of a phase 1 study of talquetamab administered in combination with subcutaneous daratumumab for relapsed or refractory multiple myeloma.

FIG. 2 shows the response rates of patients treated with daratumumab and talquetamab (cut-off date for the analysis Sep. 20, 2021). “Patients who received ≥1 study treatment and had ≥1 post-baseline response evaluation. Dara 1800 mg plus Tal (400 µg/kg weekly + 400 µg/kg and 800 µg/kg biweekly). ^{b}Dara = daratumumab; Tal = talquetamab; sCR = stringent complete response; CR = complete response; PR = partial response; VGPR = very good partial response.

FIG. 3 shows that cytokine release syndrome (CRS) was limited to grade 1 or 2 in all patients and generally confined to step-up and first full doses.^{e} The CRS was graded according to ASTCT (American Society for Transplantation and Cellular Therapy) criteria. CRS resolved in all patients.

FIG. 4 shows an increase in the frequency of CD38+ T cells following talquetamab dosing in the presence of daratumumab in patients with relapsed or refractory multiple myeloma Daratumumab (Dara) was administered weekly starting at C1D1 (Cycle 1 Day 1). ^{A}Each line represents a different patient: all patients received Dara 1800 mg = Tal (talquetamab) SC; ^{b}Step-up doses of Tal.

FIG. 5 shows the log-fold change in number of CD8+ T cells and CD4+ T cells at the indicated time points in patients with RRMM treated with 400 µg/kg of talquetamab and Daratumumab. Data from one representative subject are shown.

FIG. 6 shows the percent of CD25+ CD4+ T cells and CD25+ CD8+ T cells at the indicated time points in patients with RRMM treated with 400 µg/kg of talquetamab and 1800 mg Daratumumab. Data from one representative subject are shown.

FIGS. 7A-7D show the level of select cytokines in patients with RRMM treated with daratumumab 1800 mg plus step-up doses of talquetamab SC (10 µg/kg and 60 µg/kg) followed by 400 µg/kg of talquetamab once weekly (QW). FIG. 7A illustrates interferon gamma (IFN-g) levels. FIG. 7B illustrates tumor necrosis factor-alpha (TNF-a) levels. FIG. 7C illustrates interleukin-6 (IL-6) levels. FIG. 7D illustrates interleukin-8 (IL-8)levels. For each panel, data from one representative subject are shown.

FIG. 8 shows the duration of response in patients treated with daratumumab 1800 mg plus talquetamab (400 µg/kg SC QW or 400 µg/kg SC Q2W or 800 µg/kg SC Q2W (once every other week)). +, penta-refractory; AE, adverse event; CD38E, anti-CD38 exposed; CD38RE, refractory to anti-CD38 therapy; CR, complete response; D/C, discontinued; MR, minimal response: PD, progressive disease; PR, partial response; SC, subcutaneous; sCR, stringent complete response; SD, stable disease; TR, triple-class refractory; VGPR, very good partial response.

### DETAILED DESCRIPTION OF THE INVENTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set forth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

“About” when used in reference to numerical ranges, cutoffs, or specific values means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of an assay, result or embodiment, “about” means within one standard deviation per the practice in the art, or a range of up to 5%, whichever is larger.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA. IgD, IgE, IgG and IgM, depending on the heavy chain constant domain amino acid sequence IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen binding fragment” or “antigen binding domain” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001. WO1988/01649, WO1994/13804 and WO1992/01047.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 1. SEQ ID NO: 2 shows the extracellular domain of CD3 epsilon.

“CD38” refers to the human CD38 protein (UniProt accession no. P28907) (synonyms: ADP-ribosyl cyclase 1, cADPr hydrolase 1, cyclic ADP-ribose hydrolase 1). Human CD38 has an amino acid sequence as shown in SEQ ID NO: 3. CD38 is a single pass type II transmembrane protein with amino acid residues 1-21 representing the cytosolic domain, amino acid residues 22-42 representing the transmembrane domain, and residues 43-300 representing the extracellular domain.

“GPRC5D” refers to human G-protein coupled receptor family C group 5member D having the amino acid sequence shown in SEQ ID NO: 4.

“CH3 region” or “CH3 domain” refers to the CH3 region of an immunoglobulin. The CH3 region of human IgG1 antibody corresponds to amino acid residues 341-446. However, the CH3 region may also be any of the other antibody isotypes as described herein.

“Chimeric antigen receptor” or “CAR” refers to engineered T cell receptors which graft a ligand or antigen specificity onto T cells (for example naïve T cells central memory T cells effector memory T cells or combinations thereof). CARs are also known as artificial T- cell receptors, chimeric T-cell receptors or chimeric immunoreceptors. CARs comprise an extracellular domain capable of binding to an antigen, a transmembrane domain and at least one intracellular domain. CAR intracellular domain comprises a polypeptide known to function as a domain that transmits a signal to cause activation or inhibition of a biological process in a cell. The transmembrane domain comprises any peptide or polypeptide known to span the cell membrane and that can function to link the extracellular and signaling domains. A chimeric antigen receptor may optionally comprise a hinge domain which serves as a linker between the extracellular and transmembrane domains.

“Combination” means that two or more therapeutics are administered to a subject together in a mixture, concurrently as single agents or sequentially as single agents in any order.

“Complementarity determining regions” (CDR) are antibody regions that bind an antigen. CDRs may be defined using various delineations such as Kabat (Wu et al. J Exp Med 132: 211-50. 1970) (Kabat et al., Sequences of Proteins of Immunological Interest. 5th Ed. Public Health Service, National Institutes of Health, Bethesda, Md., 1991), Chothia (Chothia et al. J Mol Biol 196: 901-17, 1987). IMGT (Lefranc et al. Dev Comp Immunol 27: 55-77, 2003) and AbM (Martin and Thornton J Bmol Biol 263: 800-15. 1996). The correspondence between the various delineations and variable region numbering are described (see e.g., Lefranc et al. Dev Comp Immunol 27: 55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g.. Kabat; Chothia, Martin; Lefranc et al.).

TABLE 1

Kabat, IMGT, AbM, and Chothia numbering systems

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

“Comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“Enhance” or “enhanced” refers to enhancement in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g., ADCC, CDC and/or ADCP) or binding to an Fcγ receptor (FcγR) or FcRn. “Enhanced” may be an enhancement of about 10%, 20%, 30%, 40%, 50%, 60%, 70%, 80%, 90%, 100% or more, or a statistically significant enhancement.

“Fc gamma receptor” (FcyR) refers to well-known FcγRI, FcγRIIa, FcγRIIb or FcγRIII. Activating FcγR includes FcγRI. FcyRIIa and FcγRIII.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al.. (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Mutation” refers to an engineered or naturally occurring alteration in a polypeptide or polynucleotide sequence when compared to a reference sequence. The alteration may be a substitution, insertion or deletion of one or more amino acids or polynucleotides.

“Non-fixed combination” refers to separate pharmaceutical compositions of the T cell redirecting therapeutic and the anti-CD38 antibody administered as separate entities either simultaneously, concurrently or sequentially with no specific intervening time limits, wherein such administration provides effective levels of the two compounds in the body of the subject.

“Multispecific” refers to an antibody that specifically binds at least two distinct antigens or at least two distinct epitopes within the same antigen. Multispecific antibody may bind for example two, three, four or five distinct antigens or distinct epitopes within the same antigen.

When referring to a dosage amount. “µg/kg” or “mglkg” refers to the amount of an active agent, such as a bispecific antibody or antibody, in microgram (µg) or milligram (mg) administered to a subject per kilogram (kg) body weight of the subject.

“Pharmaceutical composition” refers to composition that comprises an active ingredient and a pharmaceutically acceptable carrier.

“Pharmaceutically acceptable carrier” or “excipient” refers to an ingredient in a pharmaceutical composition, other than the active ingredient, which is nontoxic to a subject.

“Philadelphia chromosome” or “Ph” refers to a well-known chromosomal translocation between chromosomes 9 and 22. resulting in the oncogenic BCR-ABL gene fusion with constitutively active tyrosine kinase activity. The translocation results in a portion of the BCR gene from chromosome 22q11 becoming fused with a portion of the ABL gene from chromosome 9q34, and is designated as t(9:22)(q34:q1 1) under the International System for Human Cytogenetic Nomenclature (ISCN). Depending on the precise location of the fusion, the molecular weight of the resulting fusion protein can range from 185 to 210 kDa. “Philadelphia chromosome” refers to all BCR-ABL fusion proteins formed due the (9:22)(q34;q 1 1) translocation.

“Recombinant” refers to DNA, antibodies and other proteins that are prepared, expressed, created or isolated by recombinant means when segments from different sources are joined to produce recombinant DNA, antibodies or proteins.

“Reduce” or “reduced” refers to a reduction in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g., ADCC, CDC and/or ADCP) or binding to an Fcγ receptor (FcγR) or FcRn. “Reduced” may be a reduction of about 10%, 20%, 30%, 40%, 50%, 60%, 70%, 80%, 90%, 100% or more, or a statistically significant enhancement. “rHuPh20” refers to recombinant human hyalurodinase having the amino acid sequence of SEQ ID NO: 37, which is a recombinant hyaluronidase (HYLENEX® recombinant) described in Int′l Pat. Pub. No. WO2004/078140.

“Refractory” refers to a cancer that is not amendable to surgical intervention and is initially unresponsive to therapy.

“Relapsed” refers to a cancer that responded to treatment but then returns.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

“GPRC5D xCD3 bispecific antibody” refers to a molecule containing two or more binding regions, wherein one of the binding regions specifically binds the cell surface antigen G Protein-Coupled Receptor Class C Group 5 Member D antigen (GPRC5D) on a target cell or tissue and wherein a second binding region of the molecule specifically binds a T cell antigen CD3. This dual/multi-target binding ability recruit T cells to the target cell or tissue leading to the eradication of the target cell or tissue.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved wellbeing of the patient.

“Treat” or “treatment” refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

The substitutions in the CH3 region are expressed as modified position(s) in the first CH3 domain of the first heavy chain/ modified position(s) in the second CH3 domain of the second heavy chain. For example, F405L/K409R refers to a F405L mutation in the first CH3 region and K09R mutation in the second CH3 region. L351Y_F405A_Y407V/T394W refers to L351Y, F40FA and Y407V mutations in the first CH3 region and T394W mutation in the second CH3 region. D399FHKRQ/K409AGRH refers to mutation in which D399 may be replaced by F, H,K R or Q, and K409 may be replaced by A, G, R or H.

Conventional one and three-letter amino acid codes are used herein as shown in Table 2.

TABLE 2

Amino acid abbreviations

| | | |
|---|---|---|
| Amino acid | Three-letter code | One-letter code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glvcine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Pomalidomide” also termed “POMALY ST®” refers to an analog of thalidomide, which is a third generation IMiD (immunomodulatory drug) with antineoplastic activity. IMiDs, such as lenalidomide and pomalidomide, form the backbone of several current multiple myeloma treatment regimens. Their exact mechanism of action is not fully understood, but IMiDs have an immunomodulatory effect on the multiple myeloma tumor microenvironment and may affect expression of tumor suppressor genes, promote apoptosis of myeloma cells, and enhance NK mediated myeloma cell lysis. The combination of daratumumab with IMiDs has been evaluated in multiple studies and demonstrated significant improvement in efficacy.

In one general aspect, the application relates to a method of treating a cancer, such as MM. preferably a refractory or relapsed MM, comprising administering to the subject a GPRC5DxCD3 bispecific antibody at a dose of 60 µg/kg to 1200 µg/kg every 1-2 weeks, and subcutaneously administering to the subject an anti-CD38 antibody at a dose of 1200 mg to 2400 mg every 1-4 weeks.

The disclosure also provides a method of killing a tumor cell in a subject in need thereof, comprising administering to the subject a GPRC5DxCD3 bispecific antibody at a dose of 60 µg/kg to 1200 µg/kg every 1-2 weeks, and subcutaneously administering to the subject an anti-CD38 antibody at a dose of 1200 mg to 2400 mg every 1-4 weeks to thereby kill the tumor cell in the subject.

The disclosure further provides a method of enhancing the activity of at least one of a GPRC5DxCD3 bispecific antibody and an anti-CD38 antibody in a subject in need thereof, comprising administering to the subject the GPRC5DxCD3 bispecific antibody at a dose of 60 µg/kg to 1200 µg/kg every 1-2 weeks, and subcutaneously administering to the subject the anti-CD38 antibody at a dose of 1200 mg to 2400 mg every 1-4 weeks to thereby kill the tumor cell in the subject.

In some embodiments, the anti-CD38 antibody is administered prior to administering the GPRC5DxCD3 bispecific antibody. For example, the anti-CD38 antibody is administered about 1, 2, 3, 4, 5, 6, 7, 8. 9. 10 hours prior to administering the GPRC5DxCD3 bispecific antibody. In certain embodiments, the anti-CD38 antibody and the GPRC5DxCD3 bispecific antibody are administered on the same day, and the anti-CD38 antibody is administered about 3 hours before the subcutaneous administration of the GPRC5DxCD3 bispecific antibody.

In some embodiments, the GPRC5DxCD3 bispecific antibody is administered weekly or biweekly.

In some embodiments, the GPRC5DxCD3 bispecific antibody and the anti-CD38 antibody are each administered to a subject having cancer, such as multiple myeloma, in an amount sufficient to alleviate or at least partially arrest the disease being treated (“therapeutically effective amount”).

In some embodiments, the GPRC5DxCD3 bispecific antibody is administered at a dose of 60 µg/kg to 1200 µg/kg weekly or biweekly. For example, the GPRCD5xCD3 bispecific antibody can be administered intravenously at a dose of 60 to 100 µg/kg, such as 60 µg/kg, 70 µg/kg, 80 µg/kg, 90 µg/kg, 100 µg/kg, or any value in-between, and the administration can be weekly, biweekly or any frequency in-between. The GPRC5DxCD3 bispecific antibody can be also administered subcutaneously at a dose of 300 to 1200 µg/kg, such as 300 µg/kg, 350 µg/kg, 400 µg/kg, 450 µg/kg, 500 µg/kg, 550 µg/kg, 600 µg/kg, 650 µg/kg, 700 µg/kg, 750 µg/kg, 800 µg/kg, 850 µg/kg, 900 µg/kg, 950 µg/kg or 1200 µg/kg, or any value in-between, and the administration can be weekly, biweekly or any frequency in between.

In some embodiments, the GPRC5DxCD3 bispecific antibody is subcutaneously administered at a dose of 400 µg/kg weekly. In some embodiments, the GPRC5DxCD3 bispecific antibody is subcutaneously administered at a dose of 800 µg/kg weekly or biweekly.

In some embodiments, the GPRC5DxCD3 bispecific antibody is subcutaneously administered at a dose of 400 µg/kg biweekly.

In some embodiments, the GPRC5DxCD3 bispecific antibody is subcutaneously administered with at least one, two or three step-up doses of 10-300 µg/kg, such as 10, 20, 30, 40, 50, 60, 70, 80, 90, 100, 110, 120, 130, 140, 150, 160, 170, 180, 190, 200, 210, 220, 230, 240. 250, 260, 270, 280, 290, or 300 µg/kg, of the GPRC5DxCD3 bispecific antibody prior to administration of the first treatment.

In some embodiment, the method further comprises subcutaneously administering to the subject step-up doses of 10 µg/kg and 60 µg/kg of the GPRC5DxCD3 bispecific antibody prior to the initial subcutaneous administration of the weight based treatment dose of 400 µg/kg of the GPRC5DxCD3 bispecific antibody. In another embodiment, the method further comprises subcutaneously administering to the subject step-up doses of 10 µg/kg, 60 µg/kg and 300 µg/kg of the GPRC5DxCD3 bispecific antibody prior to the initial subcutaneous administration of the weight based treatment dose of 800 µg/kg of the GPRC5DxCD3 bispecific antibody.

In some embodiments, the anti-CD38 antibody is subcutaneously administered at a dose of 8 mg/kg to about 16 mg/kg every 1-4 weeks. For example, the anti-CD38 antibody can be subcutaneously administered at a dose of 8 mg/kg, 8.5 mg/kg, 9 mg/kg, 9.5 mg/kg, 10 mg/kg. 10.5 mg/kg, 11 mg/kg. 11.5 mg/kg, 12, mg/kg, 12.5 mg/kg, 13 mg/kg, 13.5 mg/kg, 14 mg/kg, 14.5 mg/kg, 15 mg/kg, 15.5 mg/kg, 16 mg/kg, or any value in-between, and the administration can be once every week, every 2 weeks, every 3 weeks, or every 4 weeks, or any frequency in between.

In some embodiments, the anti-CD38 antibody is administered subcutaneously in a fixed dose of 1200 to 2400 mg every 1-4 weeks. For example, the anti-CD38 antibody can be administered subcutaneously at a dose of 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, or any value in-between, and the administration can be once every week, every 2 weeks, every 3 weeks, or every 4 weeks, or any frequency in between. In some embodiments, the anti-CD38 antibody is administered subcutaneously in a dose range of 1600 to 2000 mg every 1-4 weeks. For example, the anti-CD38 antibody can be administered subcutaneously at a dose of 1600 mg, 1650 mg, 1700 mg, 1750 mg, 1800 mg, 1850 mg, 1900 mg, 1950 mg, 2000 mg, or any value in-between, and the administration can be once every week, every 2 weeks, every 3 weeks, or every 4 weeks, or any frequency in between.

In some embodiments, the anti-CD38 antibody is administered subcutaneously at a dose of 1800 mg weekly, biweekly, once every 2 weeks, or once every 4 weeks.

Step-up doses of the GPRC5DxCD3 bispecific antibody can be administered in the initial cycle. In some embodiments, the administration of the step-up doses of the GPRC5DxCD3 bispecific antibody can be repeated after a delay in time. One or more step-up doses of the GPRC5DxCD3 bispecific antibody at a lower dosage amount can be administered to the subject prior to the initial administration of the dosage level for the weekly or biweekly treatment according to an embodiment of the application. In some embodiments, the GPRC5DxCD3 bispecific antibody is subcutaneously administered to the subject with at least one, two or three step-up doses of 5-300 µg/kg, such as 5, 10, 20, 30, 40, 50, 60, 70, 80, 90, 100, 110, 120, 130, 140, 150, 160, 170, 180, 190, 200, 210, 220, 230, 240. 250, 260, 270, 280, 290, or 300 µg/kg, or any value in-between, of the GPRC5DxCD3 bispecific antibody prior to the administration of the first weekly or biweekly treatment.

In certain embodiments, a method according to the application further comprises subcutaneously administering to the subject 5 to 100 µg/kg, such as 5, 10, 20, 30, 40. 50, 60, 70, 80, 90 or 100 µg/kg or any value in-between, of the GPRC5DxCD3 bispecific antibody before the initial subcutaneous administration of 400 µg/kg of the GPRC5DxCD3 bispecific. In other embodiments, a method according to the application further comprises subcutaneously administering to the subject 5 to 350 µg/kg, such as 5, 10, 20, 30, 40, 50, 60, 70, 80, 90, 100, 150, 200, 250, 300, or 350 µg/kg, or any value in-between, of the GPRC5DxCD3 bispecific antibody before the initial administration of 800 µg/kg of the GPRC5DxCD3 bispecific antibody. In other embodiments, the method comprises administering the first step-up dose on Day 2, the second step-up dose on Day 4, and optionally, the third step-up dose on Day 8 of the treatment.

In certain embodiments, a method of the application comprises subcutaneously administering to the subject 10 µg/kg of the GPRC5DxCD3 bispecific antibody on Day 2 of the treatment and 60 µg/kg of the GPRC5DxCD3 bispecific antibody on Day 4 of the treatment, prior to the initial subcutaneous administration of 400 µg/kg GPRC5DxCD3 bispecific antibody weekly or biweekly. In certain other embodiments, a method of the application comprises subcutaneously administering to the subject 10 µg/kg of the GPRC5DxCD3 bispecific antibody on Day 2 of the treatment, 60 µg/kg of the GPRC5DxCD3 bispecific antibody on Day 4 of the treatment, and 300 µg/kg of the GPRC5DxCD3 bispecific antibody on Day 8 of the treatment, prior to the initial subcutaneous administration of the 800 µg/kg GPRC5DxCD3 bispecific antibody weekly or biweekly.

The administration of the GPRC5DxCD3 bispecific antibody and/or the anti-CD38 antibody can be administered in 28-day cycles, and the treatment can comprise multiple cycles, such as 2, 3, 4, 5, 6, 7, 8, 9, 10 or more cycles. Repeated courses of treatment are also possible as chronic administration. The repeated administration can be at the same dose or at a different dose. For example, the GPRC5DxCD3 bispecific antibody can be subcutaneously administered at 400 µg/kg or 800 µg/kg at weekly intervals for 8 weeks, and at 400 µg/kg or 800 µg/kg biweekly for an additional period. In another example, the GPRC5DxCD3 bispecific antibody can be subcutaneously administered at 400 µg/kg or 800 µg/kg in biweekly intervals for 8 weeks, followed by an additional period of biweekly administration at the same or different dose. In other embodiments, the GPRC5DxCD3 bispecific antibody is subcutaneously administered 200 µg/kg, 300 µg/kg, or 400 µg/kg weekly, preferably 400 µg/kg weekly, for 8 weeks, followed by subcutaneous administration of 800 µg/kg GPRC5DxCD3 bispecific antibody biweekly. In other embodiments, the GPRC5DxCD3 bispecific antibody is subcutaneously administered at 400 µg/kg weekly for 8 weeks, followed by subcutaneous administration of 400 µg/kg or 800 µg/kg GPRC5DxCD3 bispecific antibody biweekly.

According to embodiments of the application, the frequency of the administration of the anti-CD38 antibody can be decreased with the time of the treatment. For example, the anti-CD38 antibody is subcutaneously administered to the subject in the dose of 1800 mg once every week during week 1 to week 8 of the treatment, once every two weeks during week 9 to week 24 of the treatment, and once every four weeks after week 24 of the treatment.

The GPRC5DxCD3 bispecific antibody and the anti-CD38 antibody can be administered by maintenance therapy, such as, e.g., once a week, 2 weeks, 3 weeks or 4 weeks, for a period of 6 months or more.

The GPRC5DxCD3 bispecific antibody and the anti-CD38 antibody can also be administered prophylactically in order to reduce the risk of developing the cancer, such as the multiple myeloma, delay the onset of the occurrence of an event in cancer progression, and/or reduce the risk of recurrence when the cancer is in remission.

In some embodiments, the GPRC5DxCD3 bispecific antibody is administered to the subject after the subject has been administered the anti-CD38 antibody. The GPRC5DxCD3 bispecific antibody and the anti-CD38 antibody can be administered on the same day. The GPRC5DxCD3 bispecific antibody can also be administered one or more days, one week, two weeks, three weeks, one month, five weeks, six weeks, seven weeks, two months, three months, four months, five months, six months, or longer after administering the anti-CD38 antibody.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is the autologous stem cell transplant (ASCT). In some embodiments, the one or more anti-cancer therapies is radiation. In some embodiments, the one or more anti-cancer therapies is surgery. In some embodiments, the one or more anti-cancer therapies is a chemotherapeutic agent. In some embodiments, the one or more anti-cancer therapies is an immunomodulatory agent. In some embodiments, the one or more anti-cancer therapies is targeted cancer therapy. In some embodiments, the one or more anti-cancer therapies is selected from the group consisting of lenalidomide, thalidomide, pomalidomide, bortezomib, carfilzomib, elotuzumab, ixazomib, melphalan, isatuximab, CELMoDs, dexamethasone, vincristine, cyclophosphamide, hydroxydaunorubicin, prednisone, rituximab, imatinib, dasatinib, nilotinib, bosutinib, ponatinib, bafetinib, saracatinib, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

In some embodiments, the one or more anti-cancer therapies is selected from the group consisting of lenalidomide, thalidomide, pomalidomide, bortezomib, carfilzomib, elotuzumab, ixazomib, melphalan, prednisone or dexamethasone, or any combination thereof.

In some embodiments, the one or more anti-cancer therapies is pomalidomide.

In some embodiments, pomalidomide is orally administered at a dose of 2 mg or 4 mg.

In some embodiments, the one or more anti-cancer therapies are pomalidomide and dexamethasone.

In some embodiments, pomalidomide is administered in a delayed dosing schedule. The delayed dosing schedule may occur in cycle 1 day 15 (ClD15) or in cycle 2 day 1 (C2Dl).

In some embodiments, pomalidomide is administered concurrently with the GPRC5DxCD3 bispecific antibody and the anti-CD38 antibody.

In some embodiments, dexamethasone is administered during at least 3 full initial IMiD-containing cycles.

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J Immunol 145:2390-6, 1990; Terhorst et al., Cell 771-80, 1981; Guse et al., Nature 398:70-3, 1999). CD38, via its NAD glycohydrolase activity, also regulates extracellular NAD⁺ levels, which have been implicated in modulating the regulatory T-cell compartment (Adriouch et al., Microbes infect 14:1284-92, 2012; Chiarugi et al., Nature Reviews 12:741-52, 2012). In addition to signaling via Ca²⁺, CD38 signaling occurs via cross-talk with antigen-receptor complexes on T- and B-cells or other types of receptor complexes. e.g., major histocompatibility complex (MHC) molecules, involving CD38 in several cellular responses, but also in switching and secretion of IgGl.

Any suitable anti-CD38 antibody can be used in a method of the application.

In some embodiments, the anti-CD38 antibody comprises the HCDR1 of SEQ ID NO: 7, the HCDR2 of SEQ ID NO:8, the HCDR3 of SEQ ID NO: 9, the LCDR1 of SEQ ID NO: 10, the LCDR2 of SEQ ID NO: 11 and the LCDR3 of SEQ ID NO: 12.

The CDRs recited above are of the Kabat numbering system. However, as provided for herein, the CDRs of the present disclosure may be provided by any appropriate numbering system, such as Kabat, Chothia, IMGT, or AbM numbering systems. Table 3 provides exemplary CDRs utilizing the Kabat, Chothia, IMGT, and AbM numbering systems:

TABLE 3

Exemplary CDRs of the anti-CD38 antibody

| | | | | |
|---|---|---|---|---|
| Region | Kabat | Chothia | AbM | IMGT |
| HCDR 1 | SFAMS (SEQ ID NO: 7) | GFTENSF (SEQ ID NO: 46) | GFTFNSFAMS (SEQ ID NO: 48) | GFTFNSFA (SEQ ID NO: 50) |
| HCDR2 | AISGSGGGTY YADSVKG (SEQ ID NO: 8) | SGSGGG (SEQ ID NO: 47) | AISGSGGGTY (SEQ ID NO: 49) | ISGSGGGT (SEQ ID NO: 51) |
| HCDR3 | DKILWFGEPVFDY (SEQ ID NO: 9) | SEQ ID NO: 9 | SEQ ID NO: 9 | AKDKILWFGEPVFDY (SEQ ID NO: 52) |
| LCDR1 | RASQSVSSYLA (SEQ ID NO: 10) | SEQ ID NO: 10 | SEQ ID NO: 10 | QSVSSY (SEQ ID NO: 53) |
| LCDR2 | DASNRAT (SEQ ID NO: 11) | SEQ ID NO: 11 | SEQ ID NO: 11 | DAS |
| LCDR3 | QOQRSNWPPT (SEQ ID NO: 12) | SEQ ID NO: 12 | SEQ ID NO: 12 | SEQ ID NO: 12 |

In some embodiments, the anti-CD38 antibody comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the anti-CD38 antibody comprises the HCDR1 of SEQ ID NO: 46, the HCDR2 of SEQ ID NO: 47, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the anti-CD38 antibody comprises the HCDR1 of SEQ ID NO: 48, the HCDR2 of SEQ ID NO: 49, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the anti-CD38 antibody comprises the HCDR1 of SEQ ID NO: 50, the HCDR2 of SEQ ID NO: 51, the HCDR3 of SEQ ID NO: 52, the LCDR1 of SEQ ID NO: 53, a LCDR2 having the amino acid sequence DAS, and the LCDR3 of SEQ ID NO: 12.

In some embodiments, the anti-CD38 antibody comprises the VH of SEQ ID NO: 5 and the VL of SEQ ID NO: 6.

In some embodiments, the anti-CD38 antibody comprises the HC of SEQ ID NO: 13 and the LC of SEQ ID NO: 14.

Other anti-CD38 antibodies used in the methods of the invention may be known antibodies, such as mAb003 described in U.S. Pat. No. 7,829,673. The VH and the VL of mAb003 may be expressed as IgG1/κ; mAb024 described in U.S. Pat. No. 7,829,673. The VH and the VL of mAb024 may be expressed as IgG1/κ; MOR-202 (MOR-03087) comprising described in U.S. Pat. No. 8,088,896. The VH and the VL of MOR-202 may be expressed as IgG1/κ; or isatuximab: described in U.S. Pat. No. 8,153,765. The VH and the VL of isatuximab may be expressed as IgG1/κ. In some embodiments, the anti-CD38 antibody comprises a) the VH of SEQ ID NO: 38 and the VL of SEQ ID NO: 39: b) the VH of SEQ ID NO: 40 and the VL of SEQ ID NO: 41; c) the VH of SEQ ID NO: 42 and the VL of SEQ ID NO: 43; or d) the VH of SEQ ID NO: 44 and the VL of SEQ ID NO: 45.

In one embodiment, the anti-CD38 antibody is DARZALEX® (daratumumab).

In some embodiments, daratumumab comprises the VH of SEQ ID NO: 5 and the VL of SEQ ID NO: 6.

In some embodiments, daratumumab comprises the HC of SEQ ID NO: 13 and the LC of SEQ ID NO: 14.

In some embodiments, the anti-CD38 antibody is chimeric, humanized or human.

In some embodiments, the anti-CD38 antibody is an IgG1, an IgG2, an IgG3 or an IgG4 isotype.

In some embodiments, the anti-CD38 antibody is an IgG1 isotype.

G Protein-Coupled Receptor Class C Group 5 Member D (GPRC5D) is a 7 transmembrane receptor protein that is classified as a type C G protein-coupled receptor based on the sequence homology score, and is an orphan receptor whose ligand and signaling mechanisms are yet to be identified. GPRC5D messenger ribonucleic acid (mRNA) is predominantly expressed in cells with a plasma cell phenotype and also expressed in all malignant plasma cells from patients with multiple myeloma. The expression of GPRC5D on the plasma cell lineage makes it a target for T cell mediated therapy to treat plasma cell disorders like multiple myeloma. A GPRC5D xCD3 bispecific antibody targets the CD3 receptor complex on T cells and GPRC5D on plasma cells. The dual binding sites allow the GPRC5DxCD3 bispecific antibody to draw CD3+ T cells in close proximity to myeloma cells, without regard to T cell receptor specificity or reliance on MHC Class 1 molecules on the surface of antigen presenting cells for activation, leading to cell death of the GPRC5D-positive cells.

Any suitable GPRC5D xCD3 bispecific antibody can be used in a method of the application. Exemplary multispecific and/or bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech), Bispecific T Cell Engager (BITE) (Micromet). Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al.. (2015) Drug Discovery Today 20(5):588-594.

In some embodiments, the GPRC5D xCD3 bispecific antibody and the anti-CD38 antibody are antigen binding fragments. Exemplary antigen binding fragments are Fab, F(ab′)2, Fd and Fv fragments.

In some embodiments, the GPRC5DxCD3 bispecific antibody is chimeric, humanized or human.

In some embodiments, the GPRC5D xCD3 bispecific antibody comprises a GPRC5D binding domain comprising a VH having the HCDR1 of SEQ ID NO: 27, the HCDR2 of SEQ ID NO: 28, the HCDR3 of SEQ ID NO: 29, and a VL having the LCDR1 of SEQ ID NO: 30. the LCDR2 of SEQ ID NO: 31 and the LCDR3 of SEQ ID NO: 32. and a CD3 binding domain comprising a VH having the HCDR1 of SEQ ID NO: 17, the HCDR2 of SEQ ID NO: 18, the HCDR3 of SEQ ID NO: 19, and a VL having the LCDR1 of SEQ ID NO: 20, the LCDR2 of SEQ ID NO: 21 and the LCDR3 of SEQ ID NO: 22. The HCDRs and LCDRs of the GPRC5D x CD3 bispecific antibody are recited in Table 4 below:

TABLE 4

Exemplary CDRs of GPRC5D x CD3 bispecific antibody

| Binding Arm | Region | Sequence | SEQ ID NO |
|---|---|---|---|
| GPRC5D | HCDR1 | GYTMN | 27 |
| | HCDR2 | LINPYNSDTNYAQKLQG | 28 |
| | HCDR3 | VALRVALDY | 29 |
| | LCDR1 | KASQNVATHVG | 30 |
| | LCDR2 | SASYRYS | 31 |
| | LCDR3 | QQYNRYPYT | 32 |
| CD3 | HCDR1 | TYAMN | 17 |
| | HCDR2 | RIRSKYNNYATYYAASVKG | 18 |
| | HCDR3 | HGNFGNSYVSWFAY | 19 |
| | LCDR1 | RSSTGAVTTSNYAN | 20 |
| | LCDR2 | GTNKRAP | 21 |
| | LCDR3 | ALWYSNLWV | 22 |

The CDRs recited in the table above are of the Kabat numbering system. However, as provided for herein, the CDRs of the present disclosure may be provided by any appropriate numbering system, such as any of the Kabat, Chothia, IMGT, or AbM numbering systems. Tables 5-7 below provide exemplary CDRs utilizing the Chothia, AbM, and IMGT numbering systems:

TABLE 5

Exemplary CDRs of GPRC5D × CD3 bispecific antibody - Chothia numbering system

| Binding Arm | Region | Sequence | SEQ ID NO |
|---|---|---|---|
| GPRC5D | HCDR1 | GYSFTGY | 54 |
| | HCDR2 | NPYNSD | 55 |
| | HCDR3 | VALRVALDY | 29 |
| | LCDR1 | KASQNVATHVG | 30 |
| | LCDR2 | SASYRYS | 31 |
| | LCDR3 | QQYNRYPYT | 32 |
| CD3 | HCDR1 | GFTFNTY | 56 |
| | HCDR2 | RSKYNNYA | 57 |
| | HCDR3 | HGNFGNSYVSWFAY | 19 |
| | LCDR 1 | RSSTGAVTTSNYAN | 20 |
| | LCDR2 | GTNKRAP | 21 |
| | LCDR3 | ALWYSNLWV | 22 |

TABLE 6

Exemplary CDRs of GPRC5D × CD3 bispecific antibody - AbM numbering system

| Binding Arm | Region | Sequence | SEQ ID NO |
|---|---|---|---|
| GPRC5D | HCDR1 | GYSFTGYTMN | 58 |
| | HCDR2 | LINPYNSDTN | 59 |
| | HCDR3 | VALRVALDY | 29 |
| | LCDR1 | KASQNVATHVG | 30 |
| | LCDR2 | SASYRYS | 31 |
| | LCDR3 | QQYNRYPYT | 32 |
| CD3 | HCDR1 | GFTFNTYAMN | 60 |
| | HCDR2 | RIRSKYNNY ATY | 61 |
| | HCDR3 | HGNFGNSYVSWFAY | 19 |
| | LCDR1 | RSSTGAVTTSNYAN | 20 |
| | LCDR2 | GTNKRAP | 21 |
| | LCDR3 | ALWYSNLWV | 22 |

TABLE 7

Exemplary CDRs of GPRC5D × CD3 bispecific antibody - IMGT numbering system

| Binding Arm | Region | Sequence | SEQ ID NO |
|---|---|---|---|
| GPRC5D | HCDR1 | GYSFTGYT | 62 |
| | HCDR2 | INPYNSDT | 63 |
| | HCDR3 | ARVALRVALDY | 64 |
| | LCDR 1 | QNVATH | 65 |
| | LCDR2 | SAS | NA |
| | LCDR3 | QQYNRYPYT | 32 |
| CD3 | HCDR1 | GFTFNTYA | 66 |
| | HCDR2 | IRSKYNNYAT | 67 |
| | HCDR3 | ARHGNFGNSYVSWFAY | 68 |
| | LCDR1 | TGAVTTSNY | 69 |
| | LCDR2 | GTN | NA |
| | LCDR3 | ALWYSNLWV | 22 |

In some embodiments, the GPRC5D ×CD3 bispecific antibody comprises a GPRC5D binding domain comprising a VH having the HCDR1 of SEQ ID NO: 27, the HCDR2 of SEQ ID NO: 28, the HCDR3 of SEQ ID NO: 29, and a VL having the LCDR1 of SEQ ID NO: 30. the LCDR2 of SEQ ID NO: 31 and the LCDR3 of SEQ ID NO: 32, and a CD3 binding domain comprising a VH having the HCDR1 of SEQ ID NO: 17, the HCDR2 of SEQ ID NO: 18, the HCDR3 of SEQ ID NO: 19, and a VL having the LCDR 1 of SEQ ID NO: 20, the LCDR2 of SEQ ID NO: 21 and the LCDR3 of SEQ ID NO: 22.

In some embodiments, the GPRC5D xCD3 bispecific antibody comprises a GPRC5D binding domain comprising a VH having the HCDR1 of SEQ ID NO: 54, the HCDR2 of SEQ ID NO: 55, the HCDR3 of SEQ ID NO: 29. and a VL having the LCDR1 of SEQ ID NO: 30, the LCDR2 of SEQ ID NO: 31 and the LCDR3 of SEQ ID NO: 32, and a CD3 binding domain comprising a VH having the HCDR1 of SEQ ID NO: 56, the HCDR2 of SEQ ID NO: 57. the HCDR3 of SEQ ID NO: 19, and a VL having the LCDR1 of SEQ ID NO: 20, the LCDR2 of SEQ ID NO: 21 and the LCDR3 of SEQ ID NO: 22.

In some embodiments, the GPRC5D ×CD3 bispecific antibody comprises a GPRC5D binding domain comprising a VH having the HCDR1 of SEQ ID NO: 58, the HCDR2 of SEQ ID NO: 59. the HCDR3 of SEQ ID NO: 29, and a VL having the LCDR1 of SEQ ID NO: 30, the LCDR2 of SEQ ID NO: 31 and the LCDR3 of SEQ ID NO: 32, and a CD3 binding domain comprising a VH having the HCDR1 of SEQ ID NO: 60, the HCDR2 of SEQ ID NO: 61, the HCDR3 of SEQ ID NO: 19, and a VL having the LCDR1 of SEQ ID NO: 20, the LCDR2 of SEQ ID NO: 21 and the LCDR3 of SEQ ID NO: 22.

In some embodiments, the GPRC5D ×CD3 bispecific antibody comprises a GPRC5D binding domain comprising a VH having the HCDR1 of SEQ ID NO: 62, the HCDR2 of SEQ ID NO: 63, the HCDR3 of SEQ ID NO: 64, and a VL having the LCDR1 of SEQ ID NO: 65. a LCDR2 having the amino acid sequence SAS, and the LCDR3 of SEQ ID NO: 32. and a CD3 binding domain comprising a VH having the HCDR1 of SEQ ID NO: 66, the HCDR2 of SEQ ID NO: 67, the HCDR3 of SEQ ID NO: 68, and a VL having the LCDR1 of SEQ ID NO: 69, a LCDR2 having the amino acid sequence GTN, and the LCDR3 of SEQ ID NO: 22.

In some embodiments, the GPRC5D ×CD3 bispecific antibody comprises a GPRC5D binding domain comprising the VH of SEQ ID NO: 33 and the VL of SEQ ID NO: 34, and a CD3 binding domain comprising the VH of SEQ ID NO: 23 and the VL of SEQ ID NO: 24.

In some embodiments, the GPRC5D ×CD3 bispecific antibody that binds GPRC5D comprises a first heavy chain (HC1) of SEQ ID NO: 35. a first light chain (LC1) of SEQ ID NO: 36. a second heavy chain (HC2) of SEQ ID NO: 25, and a second light chain (LC2) of SEQ ID NO: 26.

In some embodiments, the CD3 binding arm of the GPRC5D×CD3 bispecific antibody and the GPRC5D binding arm of the GPRC5D×CD3 bispecific antibody comprise the amino acid sequences as provided for in Tables 8a and 8b.

TABLE 8a

Sequences of CD3 binding arm of a GPRC5D×CD3 bispecific antibody

| | | | |
|---|---|---|---|
| | Region | Sequence | SEQ ID NO |
| CD3B219 | HCDR1 | TYAMN | 17 |
| | HCDR2 | RIRSKYNNYATYYAASVKG | 18 |
| | HCDR3 | HGNFGNSYVSWFAY | 19 |
| | LCDR1 | RSSTGAVTTSNYAN | 20 |
| | LCDR2 | GTNKRAP | 21 |
| | LCDR3 | ALWYSNLWV | 22 |
| | VH | EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS | 23 |
| | VL | QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP | 24 |
| | HC | EVQLVESGGGLVQPGGSLRLSCAASGFTFNTYAMNWVRQAPGKGLEWVARIRSKYNTNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDOSFLLYSKLTVDKSRWQEGNVFSCSVMHEALHNHYTQKSLSLSLGK | 25 |
| | LC | QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKA | 26 |
| | | DSSPVKAGVETTTPSKQSNNKYAASSYLSLTPEQWKSHRSYSCQVTHEGSTVEKTVAPTECS | |

TABLE 8b

Sequences of GPRC5D binding arm of a GPRC5DxCD3 bispecific antibody

| PS3B27 | Region | Sequence | SEQ ID NO |
|---|---|---|---|
| GC5B596 | HCDR1 | GYTMN | 27 |
| | HCDR2 | LINPYNSDTNYAQKLQG | 28 |
| | HCDR3 | VALRVALDY | 29 |
| | LCDR1 | KASQNVATHVG | 30 |
| | LCDR2 | SASYRYS | 31 |
| | LCDR3 | QQYNRYPYT | 32 |
| | VH | QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS | 33 |
| | VL | DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK | 34 |
| | HC | QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLD | 35 |
| | | SDOSFFLYSRLTVDKSRWQEGNVFSCSVMHEALHNHYTQKSLSLSLGK | |
| | LC | DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS | 36 |

In some embodiments, the GPRC5D×CD3 bispecific antibody can be, but are not limited to, talquetamab (also named JNJ-564 or JNJ-64407564), a GPRC5D×CD3 bispecific antibody described in Kodama et al. Mol Cancer Ther. 2019. 18(9): 1555-1564, the entire content of which is incorporated herein by reference, or a bispecific antibody that uses a human GPRC5D binding domain described in U.S. Pat. No. 10,590,196. the entire content of which is incorporated herein by reference, or a GPRC5D binding domain that competes with talquetamab or the human GPRC5D binding domain described in U.S. Pat. No. 10,590,196 for binding to human GPRC5D.

In some embodiments, talquetamab comprises a first heavy chain (HC1), a first light chain (LC1), a second heavy chain (HC2), and a second light chain (LC2), wherein the HC1 is associated with LC1 and the HC2 is associated with LC2, wherein HC1 and LC1 form a first antigen-binding site that immunospecifically binds to GPRC5D and wherein HC2 and LC2 form a second antigen-binding site that immunospecifically binds to CD3. In some embodiments, talquetamab comprises a HC1 of SEQ ID NO: 35. a LC1 of SEQ ID NO: 36, a HC2 of SEQ ID NO: 25, and a LC2 of SEQ ID NO: 26. In some embodiments, the CD3 arm and the GPRC5D arm of talquetamab form a functional bispecific antibody through an interaction between their respective Fc domains.

In some embodiments, the GPRC5D×CD3 bispecific antibody comprises any one of GPRC5D binding domains described in U.S. Pat. No. 10,900,956 or WO2020/092854 the entire content of which is incorporated herein by reference, or a GPRC5D binding domain that competes with such GPRC5D binding domain for binding to human GPRC5D.

In some embodiments, the GPRC5D×CD3 bispecific antibody is an IgG1, an IgG2, an IgG3 or an IgG4 isotype.

In some embodiments, the GPRC5D×CD3 bispecific antibody is an IgG1 isotype.

In some embodiments, the GPRC5D×CD3 bispecific antibody is an IgG2 isotype.

In some embodiments, the GPRC5D×CD3 bispecific antibody is an IgG3 isotype.

In some embodiments, the GPRC5D×CD3 bispecific antibody is an IgG4 isotype.

The GPRC5D×CD3 bispecific antibody can be of any allotype. Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host may be determined in part by the allotype of the antibody (Stickler et al., (2011) Genes and Immunity 12:213-21). Antibody allotype is related to amino acid sequence variations at specific locations in the constant region sequences of the antibody. Table 9 shows select IgG1, IgG2 and IgG4 allotypes.

TABLE 9

IgG1, IgG2 and IgG4 allotypes

| Allotype | Amino acid residue at position of diversity (residue numbering: EU Index) | | | | | | | |
|---|---|---|---|---|---|---|---|---|
| | IgG2 | | IgG4 | | IgG1 | | | |
| | 189 | 282 | 309 | 422 | 214 | 356 | 358 | 431 |
| G2m(n) | T | M | | | | | | |
| G2m(n-) | P | V | | | | | | |
| G2m(n)/(n- | T | V | | | | | | |
| nG4m(a) | | | L | R | | | | |
| G1m(17) | | | | | K | E | M | A |
| G1m(17,1) | | | | | K | D | L | A |

In some embodiments, the multispecific antibody comprises one or more Fc substitutions that reduces binding of the multispecific antibody to a Fcγ receptor (FcγR). Substitutions that reduce binding of the multispecific antibody to the FcγR reduces the Fc effector functions such as ADCC, ADCP and/or CDC of the multispecific antibody. The specific substitutions can be made in comparison to the wild-type IgG1 of SEQ ID NO: 15 or the wild-type IgG4 of SEQ ID NO: 16.

In some embodiments, the one or more Fc substitutions is selected from the group consisting of F234A/L235A on IgG4, L234A/L235A on IgG1, V234A/G237A/ P238S/H268A/V309L/A330S/P331S on IgG2, F234A/L235A on IgG4, S228P/F234A/ L235A on IgG4, N297A on all Ig isotypes, V234A/G237A on IgG2, K214T/E233P/ L234V/L235A/G236-deleted/A327G/P331 A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4 and S228P/F234A/L235A/G236-deleted/G237A/P238S on IgG4, wherein residue numbering is according to the EU index.

In some embodiments, the one or more Fc substitutions is F234A/L235A on IgG4.

In some embodiments, the one or more Fc substitutions is L234A/L235A on IgG1.

In some embodiments, the one or more Fc substitutions is V234A/G237A/ P238S/H268A/V309L/A330S/P331S on IgG2.

In some embodiments, the one or more Fc substitutions is F234A/L235A on IgG4.

In some embodiments, the one or more Fc substitutions is S228P/F234A/ L235A on IgG4.

In some embodiments, the one or more Fc substitutions is N297A on all Ig isotypes.

In some embodiments, the one or more Fc substitutions is V234A/G237A on IgG2.

In some embodiments, the one or more Fc substitutions is K214T/E233P/ L234V/L235A/G236-deleted/A327G/P331A/D365E/L358M on IgG1.

In some embodiments, the one or more Fc substitutions is H268Q/V309L/A330S/P331S on IgG2.

In some embodiments, the one or more Fc substitutions is S267E/L328F on IgG1. In some embodiments, the one or more Fc substitutions is L234F/L235E/D265A on IgG1.

In some embodiments, the one or more Fc substitutions is L234A/L235A/G237A/P238S/H268A/A330S/P331S on IgG1.

In some embodiments, the one or more Fc substitutions is S228P/F234A/L235A/G237A/P238S on IgG4 and S228P/F234A/L235A/G236-deleted/G237A/P238S on IgG4.

In some embodiments, the multispecific antibody further comprises a S228P substitution.

In some embodiments, the multispecific antibody comprises one or more asymmetric substitutions in a first CH3 domain or in a second CH3 domain, or in both the first CH3 domain and the second CH3 domain.

In some embodiments, the one or more asymmetric substitutions is selected from the group consisting of F450L/K409R, wild-type/F409L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In some embodiments, the one or more asymmetric substitutions is F450L/K409R.

In some embodiments, the one or more asymmetric substitutions is wild-type/F409L_R409K.

In some embodiments, the one or more asymmetric substitutions is T366Y/F405A.

In some embodiments, the one or more asymmetric substitutions is T366W/F405W.

In some embodiments, the one or more asymmetric substitutions is F405W/Y407A.

In some embodiments, the one or more asymmetric substitutions is T394W/Y407T.

In some embodiments, the one or more asymmetric substitutions is T394S/Y407A.

In some embodiments, the one or more asymmetric substitutions is T366W/T394S.

In some embodiments, the one or more asymmetric substitutions is F405W/T394S.

In some embodiments, the one or more asymmetric substitutions is T366W/T366S_L368A_Y407V.

In some embodiments, the one or more asymmetric substitutions is L351Y_F405A_Y407V/T394W.

In some embodiments, the one or more asymmetric substitutions is T366I_K392M_T394W/F405A_Y407V.

In some embodiments, the one or more asymmetric substitutions is T366L_K392M_T394W/F405A_Y407V.

In some embodiments, the one or more asymmetric substitutions is L351Y_Y407A/T366A_K409F.

In some embodiments, the one or more asymmetric substitutions is L351Y_Y407A/T366V_K409F.

In some embodiments, the one or more asymmetric substitutions is Y407A/T366A_K409F.

In some embodiments, the one or more asymmetric substitutions is T350V_L351Y_F405A_Y407V/T350V_T366L_K392L_T394W.

In some embodiments, the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

In some embodiments, the GPRC5D×CD3 bispecific antibody further comprises proline at position 228, alanine at position 234 and alanine at position 235 in both the HC1 and the HC2.

In some embodiments, the cancer is a hematological malignancy or a solid tumor.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt’s lymphoma (BL), a follicular lymphoma (FL), a mantle-cell lymphoma (MCL), Waldenstrom’s macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin’s lymphoma, non-Hodgkin’s lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In some embodiments, the hematological malignancy is multiple myeloma.

In some embodiments, the multiple myeloma is a newly diagnosed multiple myeloma.

In some embodiments, the multiple myeloma is a relapsed or a refractory multiple myeloma (RRMM).

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4:14)(p16:q32), t(14;16)(q32;q23), del17p, 1qAmp, t(4:14)(p16;q32) and t(14;16)(q32;q23), t(4;14)(p16:q32) and del17p, t(14:16)(q32,q23) and del17p, or t(4:14)(p16;q32), t(14;16)(q32;q23) and del17p.

In some embodiments, the subject having the high-risk multiple myeloma has one or more chromosomal abnormalities comprising: t(4;14)(p16;q32), t(14:16)(q32:q23), del17p, 1qAmp, t(4:14)(p16:q32) and t(14;16)(q32;q23), t(4;14)(p16;q32) and del17p, t(14;16)(q32;q23) and del17p; or t(4;14)(p16;q32), t(14;16)(q32;q23) and del17p, or any combination thereof.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14)(p16:q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

In some embodiments, the multiple myeloma is relapsed or refractory to treatment with an anti-CD38 antibody (e.g., daratumumab, isatuximab, etc.), lenalidomide, bortezomib, pomalidomide, carfilzomib, elotuzumab, ixazomib, melphalan or thalidomide, or any combination thereof.

In some embodiments, the multiple myeloma is relapsed or refractory to treatment with the anti-CD38 antibody. In some embodiments, the multiple myeloma is relapsed or refractory to treatment with lenalidomide. In some embodiments, the multiple myeloma is relapsed or refractory to treatment with bortezomib. In some embodiments, the multiple myeloma is relapsed or refractory to treatment with pomalidomide. In some embodiments, the multiple myeloma is relapsed or refractory to treatment with carfilzomib. In some embodiments, the multiple myeloma is relapsed or refractory to treatment with elotuzumab. In some embodiments, the multiple myeloma is relapsed or refractory to treatment with ixazomib. In some embodiments, the multiple myeloma is relapsed or refractory to treatment with melphalan. In some embodiments, the multiple myeloma is relapsed or refractory to treatment with or thalidomide.

In some embodiments, the hematological malignancy is the AML.

In some embodiments, the AML is AML with at least one genetic abnormality. In some embodiments, the AML is AML with multilineage dysplasia. In some embodiments, the AML is therapy-related AML. In some embodiments, the AML is undifferentiated AML. In some embodiments, the AML is AML with minimal maturation. In some embodiments, the AML is AML with maturation. In some embodiments, the AML is acute myelomonocytic leukemia. In some embodiments, the AML is acute monocytic leukemia. In some embodiments, the AML is acute erythroid leukemia. In some embodiments, the AML is acute megakaryoblastic leukemia. In some embodiments, the AML is acute basophilic leukemia. In some embodiments, the AML is acute panmyelosis with fibrosis. In some embodiments, the AML is myeloid sarcoma.

In some embodiments, the at least one genetic abnormality is a translocation between chromosomes 8 and 21, a translocation or an inversion in chromosome 16, a translocation between chromosomes 15 and 17, changes in chromosome 11, or mutation in fms-related tyrosine kinase 3 (FLT3), nucleophosmin (NPM1), isocitrate dehydrogenase 1(IDH1), isocitrate dehydrogenase 2 (IDH2), DNA (cytosine-5)-methyltransferase 3 (DNMT3A), CCAAT/enhancer binding protein alpha (CEBPA), U2 small nuclear RNA auxiliary factor 1(U2AF1), enhancer of zeste 2 polycomb repressive complex 2 subunit (EZH2), structural maintenance of chromosomes 1A (SMC1A) or structural maintenance of chromosomes 3 (SMC3).

In some embodiments, the at least one genetic abnormality is the translocation between chromosomes 8 and 21. In some embodiments, the at least one genetic abnormality is the translocation or an inversion in chromosome 16. In some embodiments, the at least one genetic abnormality is the translocation between chromosomes 15 and 17. In some embodiments, the at least one genetic abnormality is changes in chromosome 11. In some embodiments, the at least one genetic abnormality is the mutation in fms-related tyrosine kinase 3 (FLT3). In some embodiments, the at least one genetic abnormality is the mutation in nucleophosmin (NPM1). In some embodiments, the at least one genetic abnormality is the mutation in isocitrate dehydrogenase 1(IDH1). In some embodiments, the at least one genetic abnormality is the mutation in isocitrate dehydrogenase 2 (IDH2). In some embodiments, the at least one genetic abnormality is the mutation in DNA (cytosine-5)-methyltransferase 3 (DNMT3A). In some embodiments, the at least one genetic abnormality is the mutation in CCAAT/enhancer binding protein alpha (CEBPA). In some embodiments, the at least one genetic abnormality is the mutation in U2 small nuclear RNA auxiliary factor 1(U2AF1). In some embodiments, the at least one genetic abnormality is the mutation in enhancer of zeste 2 polycomb repressive complex 2 subunit (EZH2). In some embodiments, the at least one genetic abnormality is the mutation in structural maintenance of chromosomes 1A (SMC1A). In some embodiments, the at least one genetic abnormality is the mutation in structural maintenance of chromosomes 3 (SMC3).

In some embodiments, the at least one genetic abnormality is a translocation t(8; 21)(q22; q22), an inversion inv(16)(p13; q22), a translocation t(16; 16)(p13; q22), a translocation t(15; 17)(q22; q12), a mutation FLT3-ITD, mutations R132H or R100Q/R104V/F108L/R119Q/I130V in IDH1 or mutations R140Q or R172 in IDH2.

In some embodiments, the at least one genetic abnormality is the translocation t(8; 21)(q22; q22). In some embodiments, the at least one genetic abnormality is the inversion inv(16)(p13; q22). In some embodiments, the at least one genetic abnormality is the translocation t(16; 16)(p13; q22). In some embodiments, the at least one genetic abnormality is the translocation t(15; 17)(q22; q12). In some embodiments, the at least one genetic abnormality is the mutation FLT3-ITD. In some embodiments, the at least one genetic abnormality is the mutation R132H in IDH1. In some embodiments, the at least one genetic abnormality is the mutation R100Q/R104V/F108L/R119Q/I130V in IDH1. In some embodiments, the at least one genetic abnormality is the mutation R140Q in IDH2. In some embodiments, the at least one genetic abnormality is the mutation R172 in IDH2.

In some embodiments, the hematological malignancy is the ALL.

In some embodiments, the ALL is B-cell lineage ALL, T-cell lineage ALL, adult ALL or pediatric ALL.

In some embodiments, the ALL is B-cell lineage ALL. In some embodiments, the ALL is T-cell lineage ALL, In some embodiments, the ALL is adult ALL, In some embodiments, the ALL is pediatric ALL.

In some embodiments, the subject with ALL has a Philadelphia chromosome or is resistant or has acquired resistance to treatment with a BCR-ABL kinase inhibitor.

In some embodiments, the subject with ALL has the Philadelphia chromosome. In some embodiments, the subject with ALL is resistant or has acquired resistance to treatment with a BCR-ABL kinase inhibitor.

The Ph chromosome is present in about 20% of adults with ALL and a small percentage of children with ALL and is associated with poor prognosis. At a time of relapse, patients with Ph4+ positive ALL may be on tyrosine kinase inhibitor (TKI) regimen and may have therefore become resistant to the TKI. The anti-CD38 antibodies may thus be administered to a subject who has become resistant to selective or partially selective BCR-ABL inhibitors. Exemplary BCR-ABL inhibitors are for example imatinib, dasatinib, nilotinib, bosutinib, ponatinib, bafetinib, saracatinib, tozasertib or danusertib.

Other chromosomal rearrangements identified in B-lineage ALL patients are t(v;11q23) (MLL rearranged), t(1;19)(q23;p13.3); TCF3-PBX1 (E2A-PBX1), t(12;21)(p13;q22): ETV6-RUNX1 (TEL-AML1) and t(5;14)(q31;q32); IL3-IGH.

In some embodiments, the subject has ALL with t(v;11q23) (MLL rearranged), t(1;19)(q23:p13.3); TCF3-PBX1 (E2A-PBX1), t(12;21)(p13;q22); ETV6-RUNX1 (TEL-AML1) or t(5;14)(q31;q32); IL3-IGH chromosomal rearrangement.

Chromosomal rearrangements can be identified using well known methods, for example fluorescent in situ hybridization, karyotyping, pulsed field gel electrophoresis, or sequencing.

In some embodiments, the hematological malignancy is the smoldering multiple myeloma.

In some embodiments, the hematological malignancy is the MGUS.

In some embodiments, the hematological malignancy is the ALL.

In some embodiments, the hematological malignancy is the DLBLC.

In some embodiments, the hematological malignancy is the BL.

In some embodiments, the hematological malignancy is the FL.

In some embodiments, the hematological malignancy is the MCL.

In some embodiments, the hematological malignancy is Waldenstrom’s macroglobulinemia.

In some embodiments, the hematological malignancy is the plasma cell leukemia.

In some embodiments, the hematological malignancy is the AL.

In some embodiments, the hematological malignancy is the precursor B-cell lymphoblastic leukemia.

In some embodiments, the hematological malignancy is the precursor B-cell lymphoblastic leukemia.

In some embodiments, the hematological malignancy is the myelodysplastic syndrome (MDS).

In some embodiments, the hematological malignancy is the CLL.

In some embodiments, the hematological malignancy is the B cell malignancy.

In some embodiments, the hematological malignancy is the CML.

In some embodiments, the hematological malignancy is the HCL.

In some embodiments, the hematological malignancy is the blastic plasmacytoid dendritic cell neoplasm.

In some embodiments, the hematological malignancy is Hodgkin’s lymphoma.

In some embodiments, the hematological malignancy is non-Hodgkin’s lymphoma.

In some embodiments, the hematological malignancy is the MZL.

In some embodiments, the hematological malignancy is the MALT.

In some embodiments, the hematological malignancy is the plasma cell leukemia.

In some embodiments, the hematological malignancy is the ALCL.

In some embodiments, the hematological malignancy is leukemia.

In some embodiments, the hematological malignancy is lymphoma.

In one embodiment, the disclosure provides a method of treating a cancer in a subject, comprising administering a therapeutically effective amount of a GPRC5DxCD3 bispecific antibody to the subject to treat the cancer, wherein the subject has been treated with an anti-CD38 antibody prior to administering the GPRC5DxCD3 bispecific antibody.

The disclosure also provides a method of treating a cancer in a subject, comprising administering a therapeutically effective amount of a GPRC5DxCD3 bispecific antibody to the subject to treat the cancer, wherein the subject is relapsed or refractory to treatment with a prior anti-cancer therapeutic.

In some embodiments, the subject administered the GPRC5DxCD3 antibody is resistant and/or refractory to treatment with the anti-CD38 antibody.

In some embodiments, the cancer is a GPRC5D expressing cancer.

In some embodiments, the cancer is a hematologic malignancy.

In some embodiments, the cancer is a multiple myeloma, a smoldering myeloma, a monoclonal gammopathy of undetermined significance (MGUS), a B-cell acute lymphoblastic leukemia, a diffuse large B-cell lymphoma, a Burkitt’s lymphoma, a follicular lymphoma, a mantle-cell lymphoma, Waldenstrom’s macroglobulinemia, plasma cell leukemia, light chain amyloidosis or non-Hodgkin’s lymphoma. An experienced physician makes the cancer diagnosis.

In some embodiments, the subject is relapsed or refractory to treatment with an anti-CD38 antibody or lenalidomide, or a combination thereof.

In some embodiments, the subject is relapsed or refractory to treatment with an anti-CD38 antibody. In some embodiments, the subject is relapsed or refractory to treatment with lenalidomide.

In some embodiments, the subject is relapsed or refractory to treatment with a prior anti-cancer therapeutic, such as a therapeutic used to treat multiple myeloma or other hematological malignancies.

In some embodiments, the subject is refractory or relapsed to treatment with THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), Empliciti® (elotuzumab), SARCLISA® (isatuximab), or Alkeran® (melphalan).

In some embodiments, the subject is relapsed to treatment with DARZALEX® (daratumumab).

In some embodiments, the anti-CD38 antibody is administered or provided for administration in a pharmaceutical composition comprising between about 20 mg/mL to about 120 mg/mL of the anti-CD38 antibody in about 25 mM acetic acid, about 60 mM sodium chloride, about 140 mannitol and about 0.04% w/v polysorbate-20 (PS-20); at pH about 5.5.

In some embodiments, the anti-CD38 antibody is administered or provided for administration in a pharmaceutical composition comprising about 1,800 mg of the anti-CD38 antibody and about 30,000 U of rHuPH20.

In some embodiments, the anti-CD38 antibody is administered or provided for administration in a pharmaceutical composition comprising about 120 mg/mL of the anti-CD38 antibody and about 2,000 U/mL of rHuPH20.

In some embodiments, the anti-CD38 antibody is administered or provided for administration in a pharmaceutical composition comprising
about 5 mM and about 15 mM histidine:
about 100 mM and about 300 mM sorbitol;
about 0.01% w/v and about 0.04% w/v PS-20; and
about 1 mg/mL and about 2 mg/mL methionine, at a pH of about 5.5-5.6.

In some embodiments, the anti-CD38 antibody is administered or provided for administration in a pharmaceutical composition comprising
about 1,800 mg of the anti-CD38 antibody;
about 30,000 U of rHuPH20;
about 10 mM histidine;
about 300 mM sorbitol:
about 0.04% (w/v) PS-20; and
about 1 mg/mL methionine, at a pH of about 5.6.

In some embodiments, the anti-CD38 antibody is administered or provided for administration in a pharmaceutical composition comprising
about 120 mg/mL of the anti-CD38 antibody;
about 2,000 U/mL of rHuPH20;
about 10 mM histidine;
about 300 mM sorbitol:
about 0.04% (w/v) PS-20; and
about 1 mg/mL methionine, at a pH of about 5.6.

The invention also provides a pharmaceutical composition comprising a GPRC5DxCD3 bispecific antibody and an anti-CD38 antibody as described herein. For example, the composition can comprise a GPRC5D binding domain comprising a VH of SEQ ID NO: 33 and a VL of SEQ ID NO: 34 and a CD3 binding domain comprising the VH of SEQ ID NO: 23 and the VL of SEQ ID NO: 24, and an anti-CD38 antibody comprising a VH of SEQ ID NO: 5 and the VL of SEQ ID NO: 6.

In some embodiments, the pharmaceutical composition comprises the GPRC5DxCD3 bispecific antibody comprising the HC1 of SEQ ID NO: 35, the LC1 of SEQ ID NO: 36, the HC2 of SEQ ID NO: 25 the LC2 of SEQ ID NO: 26, and the anti-CD38 antibody comprising the HC of SEQ ID NO: 13 and the LC of SEQ ID NO: 14. In some embodiments, the GPRC5DxCD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index. In some embodiments, the GPRC5DxCD3 bispecific antibody further comprises proline at position 228, alanine at position 234 and alanine at position 235 in both the HC1 and the HC2.

The disclosure also provides a kit or a combination comprising the GPRC5DxCD3 bispecific antibody and the anti-CD38 antibody for use in a method of the application.

### Methods of Generating Antibodies Used in the Methods of the Invention

The antibodies used in the methods of the invention binding specific antigens may be selected de novo from, for example, a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-14, 1996, Sheets et al., PITAS (USA) 95:6157-62, 1998; Hoogenboom and Winter, J Mol Biol 227:381. 1991; Marks et al., J Mol Biol 222:581. 1991). Phage display libraries expressing antibody heavy and light chain variable regions as fusion proteins with bacteriophage pIX coat protein as described in Shi et al (2010) J. Mol. Biol. 397:385-96 and Int′l Pat. Pub. No. WO2009/085462. The antibody libraries may be screened for binding to the desired antigen, such as GPRC5D and the obtained positive clones may be further characterized and the Fabs isolated from the clone lysates, and subsequently cloned as full-length antibodies. Such phage display methods for isolating human antibodies are established in the art. See for example: U.S. Pat. No. 5,223,409; U.S. Pat. No. 5,403,484; U.S. Pat. No. 5,571,698; U.S. Pat. No. 5,427,908; U.S. Pat. No. 5,580,717; U.S. Pat. No. 5,969,108; U.S. Pat. No. 6,172,197; U.S. Pat. No. 5,885,793; U.S. Pat. No. 6,521,404; U.S. Pat. No. 6,544,731; U.S. Pat. No. 6,555,313; U.S. Pat. No. 6,582,915; and U.S. Pat. No. 6,593,081.

T cell redirecting bispecific antibodies may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, two monospecific bivalent antibodies are engineered to have certain substitutions at the CH3 domain that promote heterodimer stability: the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2- mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8. for example at pH of 7.0 or at pH of 7.4 may be used.

Exemplary CH3 mutations that may be used in a first heavy chain and in a second heavy chain of the bispecific antibody are K409R and/or F405L.

Additional CH3 mutations that may be used include technologies such as Duobody® mutations (Genmab), Knob-in-Hole mutations (Genentech), electrostatically-matched mutations (Chugai, Amgen. NovoNordisk. Oncomed), the Strand Exchange Engineered Domain body (SEEDbody) (EMD Serono), and other asymmetric mutations (e.g., Zymeworks).

Duobody® mutations (Genmab) are disclosed for example in US9150663 and US2014/0303356 and include mutations F405L/K409R, wild-type/F405L_R409K, T3501K370TF405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH. F405IKLSTVW/K409AGRH and Y407LWQ/K409AGRH.

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

SEEDbody mutations involve substituting select IgG residues with IgA residues to promote heavy chai heterodimerization as described in US20070287170.

Other exemplary mutations that may be used are R409D_K370E/D399K_E357K, S354C_T3(d)W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/ E356K, K253E_D282K_K322D/D239K_E240K_K292D, K392D_K409D/D356K_D399K as described in WO2007/147901, WO 2011/143545, WO2013157954, WO2013096291 and US2018/0118849.

Additional bispecific or multispecific structures that can be used as GPRC5DxCD3 bispecific antibodies include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. No. 5,932,448; U.S. Pat. No. 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine--China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

### Fc Engineering of Antibodies

The Fc region of the GPRC5DxCD3 bispecific antibodies such as bispecific or multispecific antibodies or the anti-CD38 antibodies may comprise at least one substitution in the Fc region that reduces binding of the GPRC5DxCD3 bispecific antibodies to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/ L235A on IgG4, N297A on all Ig isotypes, V234A/G237A on IgG2, K214T/E233P/ L234V/L235A/G236-deleted/A327G/P331A/D365E/L358M on IgG1, H268Q/V309L/ A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4.

Fc substitutions that may be used to reduce CDC is a K322A substitution.

Well-known S228P substitution may further be made in IgG4 antibodies to enhance IgG4 stability.

An exemplary wild-type IgG1 comprises an amino acid sequence of SEQ ID NO: 16. An exemplary wild-type IgG4 comprises an amino acid sequence of SEQ ID NO: 17.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells. For example, NK cells express FcγRIIIa. whereas monocytes express FcγRI, FcγRII and FcγRIIIa. ADCC activity of the antibodies may be assessed using an in vitro assay using cells expressing the protein the antibody binds to as target cells and NK cells as effector cells. Cytolysis may be detected by the release of label (e.g., radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

“Antibody-dependent cellular phagocytosis” (“ADCP”) refers to a mechanism of elimination of antibody-coated target cells by internalization by phagocytic cells, such as macrophages or dendritic cells. ADCP may be evaluated by using monocyte-derived macrophages as effector cells and cells that express the protein the antibody binds to as target cells also engineered to express GFP or another labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods.

“Complement-dependent cytotoxicity”, or “CDC”, refers to a mechanism for inducing cell death in which the Fc effector domain of a target-bound antibody binds and activates complement component C1q which in turn activates the complement cascade leading to target cell death. Activation of complement may also result in deposition of complement components on the target cell surface that facilitate CDC by binding complement receptors (e.g., CR3) on leukocytes. CDC of cells may be measured for example by plating Daudi cells at 1 × 10⁵ cells/well (50 µL/well) in RPMI-B (RPMI supplemented with 1% BSA), adding 50 µL of test antibodies to the wells at final concentration between 0-100 µg/mL. incubating the reaction for 15 min at room temperature, adding 11 µL of pooled human serum to the wells, and incubation the reaction for 45 min at 37° C. Percentage (%) lysed cells may be detected as % propidium iodide stained cells in FACS assay using standard methods.

Binding of the antibody to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences. San Jose, USA) for 30 min at 4° C. Cells are incubated with a test antibody on ice for 1.5 hour at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 µL of Stain Buffer containing 1:200 diluted DRAQ7 live/dead stain (Cell Signaling Technology. Danvers, USA). PE and DRAQ7 signals of the stained cells are detected by Miltenyi MACSQuant flow cytometer (Miltenyi Biotec, Auburn, USA) using B2 and B4 channel, respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

### Chimeric Antigen Receptors (CAR)

Chimeric antigen receptors (CARs) are genetically engineered receptors. These engineered receptors can be readily inserted into and expressed by immune cells, including T cells in accordance with techniques known in the art. With a CAR. a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell.

CAR typically comprises an extracellular domain that binds the antigen (e.g., prostate neoantigen or B cell maturation antigen (BCMA)), an optional linker, a transmembrane domain, and a cytosolic domain comprising a costimulatory domain and/or a signaling domain.

The extracellular domain of CAR may contain any polypeptide that binds the desired antigen (e.g., prostate neoantigen). The extracellular domain may comprise a scFv, a portion of an antibody or an alternative scaffold. CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The transmembrane domain of CAR may be derived from the transmembrane domain of CD8, an alpha, beta or zeta chain of a T-cell receptor, CD28, CD3 epsilon. CD45, CD4, CD5, CD8, CD9, CD16, CD22, CD33, CD37, CD64, CD80, CD86, CD134, CD137, CD154, KIRDS2, OX40, CD2, CD27, LFA-1 (CDI1a, CD18), ICOS (CD278), 4-1 BB (CD137), 4-1 BBL, GITR, CD40, BAFFR, HVEM (LIGHTR), SLAMF7, NKp80 (KLRFI), CD160, CD1 9, IL2R beta, IL2R gamma, IL7Ra, IRGA1, VLA1, CD49a, ITGA4, IA4, CD49D, ITGA6, VLA-6, CD49f, ITGAD, CDI Id, ITGAE, CD103, ITGAL, CDI 1a, LFA-1, ITGAM, CDI1b, ITGAX, CDI1c, ITGB1, CD29, ITGB2, CD1 8, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

The intracellular costimulatory domain of CAR may be derived from the intracellular domains of one or more co-stimulatory molecules. Co-stimulatory molecules are well-known cell surface molecules other than antigen receptors or Fc receptors that provide a second signal required for efficient activation and function of T lymphocytes upon binding to antigen. Exemplary co-stimulatory domains that can be used in CARs are intracellular domains of 4-1BB, CD2, CD7. CD27. CD28. CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, and ZAP70.

The intracellular signaling domain of CAR may be derived from the signaling domains of for example O′O3ζ, CD3ε, CD22, CD79a, CD66d or CD39. “Intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

The optional linker of CAR positioned between the extracellular domain and the transmembrane domain may be a polypeptide of about 2 to 100 amino acids in length. The linker can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof The linker may also be derived from a hinge region or portion of the hinge region of any immunoglobulin.

Exemplary CARs that may be used are for example CAR that contains an extracellular domain that binds the prostate neoantigen of the invention. CD8 transmembrane domain and CD3ζ signaling domain. Other exemplary CARs contain an extracellular domain that binds the prostate neoantigen of the invention, CD8 or CD28 transmembrane domain, CD28, 41BB or OX40 costimulatory domain and CD3ζ signaling domain.

CARs are generated by standard molecular biology techniques. The extracellular domain that binds the desired antigen may be derived from antibodies or their antigen binding fragments generated using the technologies described herein.

### Outcomes

In some embodiments, the subject treated by the methods provided for herein has a partial response (PR) or better. In some embodiments, the subject treated by the methods provided for herein has a very good partial response (VGPR) or better. In some embodiments, the subject treated by the methods provided for herein has a complete response (CR) or better. In some embodiments, the subject treated by the methods provided for herein has a stringent complete response (sCR) or better. In some embodiments, PR, VGPR, CR. and sCR are as defined by the IMWG 2016 criteria. In some embodiments, PR is defined as having a greater than 50% reduction of serum M-protein and reduction in 24 hours urinary M-protein by >90% or to <200 mg/24 hours. In some embodiments, VGPR is defined as having a serum and urine M-protein level detectable by immunofixation but not on electrophoresis or > 90% reduction in serum M-protein plus urine M-protein level <100 mg/24 h. In some embodiments, CR is defined as having a negative immunofixation on serum and urine and disappearance of any soft tissue plasmacytomas and < 5% plasma cells in bone marrow. In some embodiments, sCR is defined as the CR definition as above plus normal FLC ratio and absence of clonal cells in bone marrow by immunohistochemistry or immunofluorescence.

In some embodiments, treatment via the methods provided for herein will result in T-cell activation. In some embodiments, the T-cell activation results in an increase in at least one of CD25, PD-1, CD38 on CD4+ T cells, CD38 on CD8+ T cells, or any combination thereof. In some embodiments, the T-cell activation results in an increase in CD25. In some embodiments, the T-cell activation results in an increase in PD-1. In some embodiments, the T-cell activation results in an increase in CD38 on CD4+ T cells. In some embodiments, the T-cell activation results in an increase in or CD38 on CD8+ T cells. In some embodiments, treatment via the methods provided for herein will result in an increase in the frequency of at least one of CD38+ CD8+T cells. CD38+ CD4+ T cells, Tregs T cells, or any combination thereof. In some embodiments, treatment via the methods provided for herein will result in an increase in the frequency of CD38+ CD8+ T cells. In some embodiments, treatment via the methods provided for herein will result in an increase in the frequency of CD38+ CD4+ T cells. In some embodiments, treatment via the methods provided for herein will result in an increase in the frequency of Tregs T cells.

In some embodiments, the methods provided for herein result in an enhanced activity of, or results in an increased efficacy of the components of the method when administered as monotherapies. In some embodiments, treatment via the methods provided for herein results in enhanced activity of the GPRC5DxCD3 bispecific antibody as compared to a treatment without the anti-CD38 antibody. In some embodiments, treatment via the methods provided for herein results in enhanced activity of the anti-CD38 antibody as compared to a treatment without the GPRC5DxCD3 bispecific antibody.

### Numbered Embodiments

The present disclosure also provides the following numbered embodiments:
1. A method of treating a cancer in a subject in need thereof, comprising:
   (1) administering to the subject a GPRC5DxCD3 bispecific antibody at a dose of 60 µg/kg to 1200 µg/kg every 1-2 weeks, and
   (2) subcutaneously administering to the subject an anti-CD38 antibody at a dose of 1200 mg to 2400 mg every 1-4 weeks.
   1a. A method of treating a cancer in a subject in need thereof, comprising:
      (1) intravenously administering to the subject a GPRC5DxCD3 bispecific antibody at a dose of 60 µg/kg to 1200 µg/kg every 1-2 weeks, and
      (2) subcutaneously administering to the subject an anti-CD38 antibody at a dose of 1200 mg to 2400 mg every 1-4 weeks.
      1a1. The method of embodiment 1a, wherein the GPRC5DxCD3 bispecific antibody is intravenously administered to the subject at a dose of 60 µg/kg weekly.
      1a2. The method of embodiment 1a or 1a1, wherein the anti-CD38 antibody is subcutaneously administered at a dose of 1800 mg weekly, biweekly, every three weeks or every four weeks.
      1a3. The method of embodiment 1a1, wherein the anti-CD38 antibody is subcutaneously administered at a dose of 1800 mg once every week during week 1 to week 8 of the treatment, once every two weeks during week 9 to week 24 of the treatment, and once every four weeks after week 24 of the treatment.
2. The method of embodiment 1, comprising:
   (1) subcutaneously administering to the subject the GPRC5DxCD3 bispecific antibody at a dose of 300 µg/kg to 1200 µg/kg every 1-2 weeks, and
   (2) subcutaneously administering to the subject the anti-CD38 antibody at a dose of 1600 mg to 2000 mg every 1-4 weeks.
3. The method of embodiment 2. further comprising subcutaneously administering to the subject the GPRC5DxCD3 bispecific antibody at a dose lower than that used in step (1) prior to step (1).
   3a. The method of embodiment 3. wherein the GPRC5DxCD3 bispecific antibody at the dose lower than that used in step (1) is subcutaneously administered to the subject after the initial administration of the anti-CD38 antibody, preferably the GPRC5DxCD3 bispecific antibody at the dose lower than that used in step (1) is initially administered at least 20 hours after the initial administration of the anti-CD38 antibody.
   3b. The method of embodiment 3 or 3a, wherein the GPRC5DxCD3 bispecific antibody is subcutaneously administered to the subject at a dose of 5 to 100 µg/kg, such as 5, 10, 20, 30, 40, 50, 60, 70, 80, 90 or 100 µg/kg or any value in-between, prior to the initial subcutaneous administration of 400 µg/kg of the GPRC5DxCD3 bispecific antibody in step (1).
   3c. The method of embodiment 3b, wherein at least two step-up doses of the GPRC5DxCD3 are administered, preferably the first step-up dose is administered on Day 2 of the treatment, and the second step-up dose is administered on Day 4 of the treatment.
   3d. The method of embodiment 3c, wherein the GPRC5DxCD3 bispecific antibody is subcutaneously administered to the subject at a dose of 10 µg/kg, such as on day 2 of the treatment, and at a dose of 60 µg/kg, such as on day 4 of the treatment, prior to the initial subcutaneous administration of 400 µg/kg of the GPRC5DxCD3 bispecific antibody in step (1).
   3e. The method of any one of embodiments 3 to 3d, wherein the GPRC5DxCD3 bispecific antibody is subcutaneously administered to the subject at a dose of 5 to 350 µg/kg, such as 5, 10, 20, 30, 40, 50, 60, 70, 80, 90, 100, 150, 200, 250, 300, or 350 µg/kg, or any value in-between, prior to the initial subcutaneous administration of 800 µg/kg of the GPRC5DxCD3 bispecific antibody in step (1).
   3f. The method of embodiments 3e, wherein at least three step-up doses of the GPRC5DxCD3 are administered, preferably the first step-up dose is administered on Day 2 of the treatment, the second step-up dose is administered on Day 4 of the treatment, and the third step-up dose is administered on Day 8 of the treatment.
   3g. The method of embodiment 3f, wherein the GPRC5DxCD3 bispecific antibody is subcutaneously administered to the subject at a dose of 10 µg/kg. such as on day 2 of the treatment, at a dose of 60 µg/kg, such as on day 4 of the treatment, and at a dose of 300 µg/kg, such as on day 8 of the treatment, prior to the initial subcutaneous administration of 800 µg/kg of the GPRC5DxCD3 bispecific antibody in step (1).
4. The method of any one of embodiments 2 to 3g, wherein the GPRC5DxCD3 bispecific antibody is subcutaneously administered to the subject at a dose of 300 µg/kg, 400 µg/kg. 450 µg/kg. 500 µg/kg, 550 µg/kg, 600 µg/kg, 700 µg/kg, 750 µg/kg, 800 µg/kg. 850 µg/kg. 900 µg/kg. 950 µg/kg, 1000 µg/kg, 1050 µg/kg, 1200 µg/kg, or any dose in-between, once every week or once every two weeks.
   4a. The method of embodiment 4, wherein the GPRC5DxCD3 bispecific antibody is subcutaneously administered to the subject at a dose of 300 µg/kg, 400 µg/kg, 450 µg/kg, 500 µg/kg. 550 µg/kg. 600 µg/kg. 700 µg/kg, 750 µg/kg, 800 µg/kg, 850 µg/kg, 900 µg/kg, 950 µg/kg, 1000, 1050 µg/kg, or 1200 µg/kg weekly.
   4b. The method of embodiment 4, wherein the GPRC5DxCD3 bispecific antibody is subcutaneously administered to the subject at a dose of 300 µg/kg, 400 µg/kg, 450 µg/kg, 500 µg/kg, 550 µg/kg, 600 µg/kg, 700 µg/kg, 750 µg/kg, 800 µg/kg, 850 µg/kg, 900 µg/kg, 950 µg/kg, 1000 µg/kg, 1050 µg/kg, or 1200 µg/kg biweekly.
5. The method of embodiment 4. wherein the GPRCSDxCD3 bispecific antibody is subcutaneously administered to the subject at a dose of 400 µg/kg weekly, or 400 µg/kg biweekly, or 800 µg/kg biweekly.
6. The method of embodiment 5, wherein the GPRC5DxCD3 bispecific antibody is subcutaneously administered to the subject at a dose of 400 µg/kg weekly.
   6a. The method of embodiment 5, wherein the GPRC5DxCD3 bispecific antibody is subcutaneously administered to the subject at a dose of 400 µg/kg biweekly.
   6b. The method of embodiment 5. wherein the GPRC5DxCD3 bispecific antibody is subcutaneously administered to the subject in a dose 400 µg/kg biweekly, or 800 µg/kg biweekly.
   6c. The method of embodiment 5, wherein the GPRC5DxCD3 bispecific antibody is subcutaneously administered to the subject in a dose 800 µg/kg biweekly.
   6d. The method of embodiment 5, wherein the GPRC5DxCD3 bispecific antibody is subcutaneously administered to the subject at a dose of 400 µg/kg weekly for the first 8 weeks, followed by biweekly administration of the GPRC5DxCD3 bispecific antibody at a dose of 800 µg/kg.
   6e. The method of embodiment 5, wherein the GPRC5DxCD3 bispecific antibody is subcutaneously administered to the subject at a dose of 400 µg/kg weekly for 8 weeks, followed by biweekly administration of the GPRC5DxCD3 bispecific antibody at a dose of 400 µg/kg.
7. The method of any one of embodiments 1 to 6e, wherein the anti-CD38 antibody is subcutaneously administered to the subject at the dose of 1800 mg once every week during week 1 to week 8 of the treatment, once every two weeks during week 9 to week 24 of the treatment, and once every four weeks after week 24 of the treatment.
   7a. The method of any one of embodiments 1 to 6e, wherein the anti-CD38 antibody is subcutaneously administered to the subject at the dose of 1800 mg once every week.
   7b. The method of any one of embodiments 1 to 6e, wherein the anti-CD38 antibody is subcutaneously administered to the subject at the dose of 1800 mg once every two weeks.
   7c. The method of any one of embodiments 1 to 6e, wherein the anti-CD38 antibody is subcutaneously administered to the subject at the dose of 1800 mg once every three weeks.
   7d. The method of any one of embodiments 1 to 6e, wherein the anti-CD38 antibody is subcutaneously administered to the subject at the dose of 1800 mg once every four weeks.
8. The method of any one of embodiments 1 to 7d. wherein the anti-CD38 antibody is administered or provided for administration together with rHuPH20, such as about 30,000 U of rHuPH20.
   8a. The method of any one of embodiments 1 to 7d. further comprising administering to the subject rHuPH20 to decrease the injection volume required, facilitating the subcutaneous administration of the anti-CD38 antibody.
   8b. The method of embodiment 8a, wherein the rHuPH20 is subcutaneously administered together with the anti-CD38 antibody.
   8c. The method of embodiment 8a. wherein the rHuPH20 is subcutaneously administered separately from the anti-CD38 antibody.
   8d. The method of any one of embodiments 8a to 8d. wherein the rHuPH20 is subcutaneously administered at a dose of 10,000 - 50,000 U, such as 10,000, 20,000, 30,000, 40,000 or 50,000 U, or any value in-between.
   8e. The method of any one of embodiments 8a to 8d, wherein the rHuPH20 is subcutaneously administered at a dose of 30,000U.
   8d. The method of any one of embodiments 8 to 8e, wherein the rHuPH20 and the anti-CD38 antibody are administered together in the same pharmaceutical composition.
9. The method of any one of embodiments 1 to 8d, wherein the GPRC5DxCD3 bispecific antibody comprises:
   (i) a GPRC5D binding domain comprising a heavy chain variable region (VH) having heavy chain complementarity determining regions (HCDRs) HCDR1, HCDR2 and HCDR3 of the amino acid sequences of SEQ ID NO: 27, SEQ ID NO: 28, and SEQ ID NO: 29. respectively, and a light chain variable region (VL) having light chain complementarity determining regions (LCDRs) LCDR1, LCDR2 and LCDR3 of the amino acid sequences of SEQ ID NO: 30, SEQ ID NO: 31, and SEQ ID NO: 32, respectively, and
   (j) a CD3 binding domain comprising a VH having HCDR1, HCDR2 and HCDR3 of the amino acid sequences of SEQ ID NO: 17, SEQ ID NO: 18, and SEQ ID NO: 19, respectively, and a VL having LCDR1, LCDR2 and LCDR3 of the amino acid sequences of SEQ ID NO: 20, SEQ ID NO: 21, and SEQ ID NO: 22, respectively.
   9a. The method of embodiment 9, wherein the GPRC5D binding domain comprises the VH having the amino acid sequence of SEQ ID NO: 33 and the VL having the amino acid sequence of SEQ ID NO: 34; the CD3 binding domain comprises the VH having the amino acid sequence of SEQ ID NO: 23 and the VL having the amino acid sequence of SEQ ID NO: 24.
10. The method of embodiment 9a. wherein the GPRC5DxCD3 bispecific antibody comprises a first heavy chain (HC1) having the amino acid sequence of SEQ ID NO: 35, a first light chain (LC1) having the amino acid sequence of SEQ ID NO: 36, a second heavy chain (HC2) having the amino acid sequence of SEQ ID NO: 25, and a second light chain (LC2) having the amino acid sequence of SEQ ID NO: 26.
11. The method of any one of embodiments 1 to 8d, wherein the GPRC5DxCD3 bispecific antibody comprises talquetamab (also named JNJ-564 or JNJ-64407564), a GPRC5DxCD3 bispecific antibody described in Kodama et al. Mol Cancer Ther. 2019. 18(9): 1555-1564, or a bispecific antibody that uses a GPRC5D human binding domain described in U.S. Pat. No. 10,590,196, 10,906,956 or WO2020/092854, or a GPRC5D binding domain that competes with talquetamab or such GPRC5D binding domain for binding to human GPRC5D.
   11a. The method of any one of embodiments 1 to 8d, wherein the GPRCSDxCD3 bispecific antibody comprises an antigen binding fragment, such as Fab. F(ab′)2, Fd or Fv fragment.
   11b. The method of any one of embodiments 1 to 8d, wherein the GPRC5DxCD3 bispecific antibody is chimeric, humanized or human.
   11c. The method of any one of embodiments 1 to 8d, wherein the GPRC5DxCD3 bispecific antibody is an IgG1, an IgG2, an IgG3 or an IgG4 isotype.
   11d. The method of any one of embodiments 1 to 8d, wherein the GPRC5DxCD3 bispecific antibody is an IgG4 isotype.
12. The method of any one of embodiments 1 to 11d, wherein the anti-CD38 antibody comprises a VH having HCDR1, HCDR2 and HCDR3 of the amino acid sequences of SEQ ID NO: 7. SEQ ID NO: 8, and SEQ ID NO: 9, respectively, and a VL having LCDR1, LCDR2 and LCDR3 of the amino acid sequences of SEQ ID NO: 10, SEQ ID NO: 11, and SEQ ID NO: 12, respectively.
   12a. The method of any one of embodiments 1 to 11d, wherein the anti-CD38 antibody comprises a) the VH of SEQ ID NO: 38 and the VL of SEQ ID NO: 39; b) the VH of SEQ ID NO: 40 and the VL of SEQ ID NO: 41; c) the VH of SEQ ID NO: 42 and the VL of SEQ ID NO: 43; or d) the VH of SEQ ID NO: 44 and the VL of SEQ ID NO: 45.
   12b. The method of any one of embodiments 1 to 11d, wherein the CD38 antibody comprises the VH having the amino acid sequence of SEQ ID NO: 5, and the VL having the amino acid sequence of SEQ ID NO: 6.
13. The method of any one of embodiments 1 to 11d, wherein the anti-CD38 antibody is selected from the group consisting of mAb003 described in U.S. Pat. No. 7,829,673; mAb024 described in U.S. Pat. No. 7,829,673; MOR-202 (MOR-03087) described in US. Pat. No. 8,088,896; or isatuximab described in U.S. Pat. No. 8,153,765, and daratumumab.
   13a. The method of any one of embodiments 1 to 11d, wherein the anti-CD38 antibody comprises the HC of SEQ ID NO: 13 and the LC of SEQ ID NO: 14.
   13b. The method of any one of embodiments 1 to 11d, wherein the anti-CD38 antibody is chimeric, humanized or human.
   13c. The method of any one of embodiments 1 to 11d, wherein the anti-CD38 antibody is an IgG1, an IgG2, an IgG3 or an IgG4 isotype.
   13d. The method of any one of embodiments 1 to 11a, wherein the anti-CD38 antibody is an IgG1 isotype.
   13e. The method of any one of embodiments 1 to 13d, wherein the GPRC5DxCD3 bispecific antibody and/or the anti-CD38 antibody comprises one or more Fc substitutions described herein.
   13f. The method of embodiment 13e, wherein the Fc substitution is selected from the group consisting of F234A/L235A on IgG4, L234A/L235A on IgG1, V234A/G237A/ P238S/H268A/V309L/A330S/P331S on IgG2, F234A/L235A on IgG4, S228P/F234A/L235A on IgG4, N297A on all Ig isotypes, V234A/G237A on IgG2, K214T/E233P/ L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4 and S228P/F234A/L235A/G236-deleted/G237A/P238S on IgG4, wherein residue numbering is according to the EU index.
   13g. The method of embodiment 13e, wherein the Fc substitution is selected from the group consisting of:
      (1) F234A/L235A on IgG4;
      (2) L234A/L235A on IgG1;
      (3) V234A/G237A/ P238S/H268A/V309L/A330S/P331S on IgG2;
      (4) F234A/L235A on IgG4;
      (5) S228P/F234A/ L235A on IgG4;
      (6) N297A on all Ig isotypes;
      (7) V234A/G237A on IgG2;
      (8) K214T/E233P/ L234V/L235A/G236-deleted/A327G/P331A/D365E/L358M on IgG1;
      (9) H268Q/V309L/A330S/P331S on IgG2;
      (10) S267E/L328F on IgG1;
      (11) L234F/L235E/D265A on IgG1;
      (12) L234A/L235A/G237A/P238S/H268A/A330S/P331S on IgG1;
      (13) S228P/F234A/L235A/G237A/P238S on IgG4 and S228P/F234A/L235A/G236-deleted/G237A/P238S on IgG4; and/or
      (14) S228P substitution.
   13h. The method of any one of embodiments 1 to 13e, wherein the GPRC5DxCD3 bispecific antibody comprises one or more asymmetric substitutions in a first CH3 domain or in a second CH3 domain, or in both the first CH3 domain and the second CH3 domain.
   13i. The method of embodiment 13h, wherein the one or more asymmetric substitutions is selected from the group consisting of F450L/K409R, wild-type/F409L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.
   13j. The method of any one of embodiments 1 to 13e. wherein the GPRC5DxCD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.
   13k. The method of embodiment 13j, wherein the GPRC5DxCD3 bispecific antibody further comprises proline at position 228, alanine at position 234 and alanine at position 235 in both the HC1 and the HC2.
14. The method of any one of embodiments 1 to 13k, wherein the cancer comprises a hematological malignancy or a solid tumor.
   14a. The method of embodiment 14, wherein the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt’s lymphoma (BL), a follicular lymphoma (FL), a mantle-cell lymphoma (MCL), Waldenstrom’s macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin’s lymphoma, non-Hodgkin’s lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma
   14b. The method of embodiment 14a, wherein the hematological malignancy comprises multiple myeloma.
   14c. The method of embodiment 14b, wherein the multiple myeloma is a newly diagnosed multiple myeloma.
   14d. The method of embodiment 14b, wherein the multiple myeloma is a relapsed or a refractory multiple myeloma (RRMM).
   14e. The method of embodiment 14b, wherein the multiple myeloma is a high-risk multiple myeloma.
   14f. The method of embodiment 14b, wherein the subject having the high-risk multiple myeloma has one or more chromosomal abnormalities comprising: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p; or t(4;14)(p16;q32), t(14;16)(q32;q23) and del 17p, or any combination thereof.
   14g. The method of any one of embodiments 1 to 14f, wherein the cancer comprises multiple myeloma that is relapsed or refractory to a treatment.
   14h. The method of embodiment 14g, wherein the cancer comprises multiple myeloma that is relapsed or refractory to a treatment with the anti-CD38 antibody, lenalidomide, bortezomib, pomalidomide, carfilzomib, isatuximab, elotuzumab, ixazomib, melphalan or thalidomide, or any combination thereof.
   14i. The method of any one of embodiments 1 to 14, wherein the cancer comprises AML.
   14j. The method of embodiment 14i, wherein the AML is AML with at least one genetic abnormality, AML with multilineage dysplasia, therapy-related AML, undifferentiated AML. AML with minimal maturation, AML with maturation, acute myelomonocytic leukemia, acute monocytic leukemia, acute erythroid leukemia, acute megakaryoblastic leukemia, acute basophilic leukemia, acute panmyelosis with fibrosis or myeloid sarcoma.
   14k. The method of any one of embodiments 1 to 14, wherein the cancer comprises any GPRC5D expressing cancer, such as multiple myeloma, a smoldering myeloma, a monoclonal gammopathy of undetermined significance (MGUS), a B-cell acute lymphoblastic leukemia, a diffuse large B-cell lymphoma, a Burkitt’s lymphoma, a follicular lymphoma, a mantle-cell lymphoma, Waldenstrom’s macroglobulinema, plasma cell leukemia, light chain amyloidosis or non-Hodgkin’s lymphoma.
   14l. The method of any one of embodiments 1 to 14. wherein the subject is relapsed or refractory to treatment with the anti-CD38 antibody or lenalidomide, or a combination thereof.
   14m. The method of any one of embodiments 1 to 14, wherein the subject is refractory or relapsed to treatment with THALOMID® (thalidomide), REVLIMID® (lenalidomide), POMALYST® (pomalidomide), VELCADE® (bortezomib), NINLARO (ixazomib), KYPROLIS®(carfilzomib). FARADYK® (panobinostat), AREDIA® (pamidronate), ZOMETA® (zoledronic acid), DARZALEX® (daratumumab), Empliciti® (elotuzumab), SARCLISA® (isatuximab), or Alkeran® (melphalan).
   14m. The method of any one of embodiments 1 to 14, wherein the subject is refractory or relapsed to treatment with THALOMID® (thalidomide), REVLIMID® (lenalidomide), POMALYST® (pomalidomide), VELCADE® (bortezomib), NINLARO (ixazomib), KYPROLIS®(carfilzomib). FARADYK® (panobinostat), AREDIA® (pamidronate), ZOMETA® (zoledronic acid), DARZALEX® (daratumumab), Empliciti® (elotuzumab), SARCLISA® (isatuximab), or Alkeran® (melphalan).
   14n. The method of any one of embodiments 1 to 14, wherein the subject is relapsed to treatment with DARZALEX® (daratumumab).
15. A method of treating multiple myeloma in a subject in need thereof, comprising:
   (1) subcutaneously administering to the subject at least one of 400 µg/kg of a GPRC5DxCD3 bispecific antibody weekly or 800 µg/kg of a GPRC5DxCD3 bispecific antibody biweekly, and
   (2) subcutaneously administering to the subject 1800 mg of an anti-CD38 antibody once every week during week 1 to week 8 of the treatment, once every two weeks during week 9 to week 24 of the treatment, and once every four weeks after week 24 of the treatment,
   wherein the GPRC5DxCD3 bispecific antibody comprises a first heavy chain (HC1) of SEQ ID NO: 35, a first light chain (LC1) of SEQ ID NO: 36, a second heavy chain (HC2) of SEQ ID NO: 25, and a second light chain (LC2) of SEQ ID NO: 26, and the anti-CD38 antibody comprises the HC of SEQ ID NO: 13 and the LC of SEQ ID NO: 14.
16. The method of embodiment 15, further comprising subcutaneously administering to the subject 10 to 300 µg/kg of the GPRC5DxCD3 bispecific antibody, prior to the initial subcutaneous administration of the 400 µg/kg or 800 µg/kg GPRC5DxCD3 bispecific antibody.
17. The method of any one of embodiments 14 to 16, wherein the subject has received at least one prior treatment of multiple myeloma, preferably, the subject is relapsed or refractory to the at least one prior treatment, more preferably, the prior treatment comprises at least one of a proteasome inhibitor (PI) and an immunomodulatory agent (IMiD).
18. The method of embodiment 17, wherein the subject is refractory or relapsed to a treatment selected from the group consisting of an anti-CD38 antibody, lenalidomide, bortezomib, pomalidomide, carfilzomib, elotuzumab, ixazomib, isatuximab, melphalan and thalidomide, or any combination thereof, preferably, the subject is lenalidomide refractory.
19. The method of any one of embodiments 1 to 18, further comprising administering to the subject another treatment, such as pomalidomide and/or dexamethasone.
20. The method of any one of embodiments 1 to 19, wherein the treatment results in T-cell activation, such as an increase in at least one of CD25, PD-1, CD38 on CD4+ and CD8+ T cells.
21. The method of any one of embodiments 1 to 19, wherein the treatment results in an increase in frequency of at least of CD38+ CD8+ T cells, CD38+ CD4+ T cells and Tregs T cells.
22. The method of any one of embodiments 1 to 19, wherein the treatment results in enhanced activity of the GPRC5DxCD3 bispecific antibody compared to a treatment without the anti-CD38 antibody.
23. The method of any one of embodiments 1 to 19, wherein the treatment results in enhanced activity of the anti-CD38 antibody compared to a treatment without the GPRC5DxCD3 bispecific antibody.
24. A GPRC5DxCD3 bispecific antibody for use in treating a cancer in a subject in need thereof using a method of any one of embodiments 1 to 23.
25. An anti-CD38 antibody for use in treating a cancer in a subject in need thereof using a method of any one of embodiments 1 to 23.
26. A combination of a GPRC5DxCD3 bispecific antibody and an anti-CD38 antibody for use in treating a cancer in a subject in need thereof using a method of any one of embodiments 1 to 23.
27. Use of a GPRC5DxCD3 bispecific antibody in the manufacture of a medicament for treating a cancer in a subject in need thereof using a method of any one of embodiments 1 to 23.
28. Use of an anti-CD38 antibody in the manufacture of a medicament for treating a cancer in a subject in need thereof using a method of any one of embodiments 1 to 23.
29. Use of a combination of a GPRC5DxCD3 bispecific antibody and an anti-CD38 antibody in the manufacture of a medicament for treating a cancer in a subject in need thereof using a method of any one of embodiments 1 to 23.
30. A kit comprising a GPRC5DxCD3 bispecific antibody, an anti-CD38 antibody and instructions on using the antibodies in treating a cancer in a subject in need thereof using a method of any one of embodiments 1 to 23.

While having described the invention in general terms, the embodiments of the invention will be further disclosed in the following examples that should not be construed as limiting the scope of the claims.

### EXAMPLES

The following examples are provided to further describe some of the embodiments disclosed herein. The examples are intended to illustrate, not to limit, the disclosed embodiments.

### General Materials and Methods

### Antibodies and Reagents

Anti-GPRC5D/anti-CD3 antibody JNJ-564 (also termed JNJ-64407564, talquetamab) and daratumumab were made by Janssen Pharmacetuicals. 3930 (IgG isotype control), GPFC5DxNull control, and 7008 (NullxCD3 control) were all made by Janssen Pharmaceuticals and were used as control antibodies.

JNJ-564 comprises a CD3 binding arm CD3B219 and a GPRC5D binding arm GC5B596, the amino acid sequences of which are shown in Table 8a and Table 8b, respectively.

### Bone Marrow and Peripheral Blood Mononuclear Cells

Peripheral blood mononuclear cells (PBMCs) from healthy donors and MM patients, and bone marrow mononuclear cells (BM-MNCs) from MM patient BM aspirates were isolated by Ficoll-Hypaque density-gradient centrifugation.

### Flow Cytometric Analysis of Bone Marrow and Blood Samples From MM Patients

BM-localized MM cells were identified and analysed for cell surface marker expression levels by staining 1.0×10⁶ cells/mL with HuMax-003 (CD38) FITC (this antibody binds to an epitope distinct from the epitope bound by daratumumab, Janssen Pharmaceuticals), CD138 PE, CD56 PC7, CD45 Krome Orange (all Beckman Coulter), CD269 (BCMA) APC (Biolegend), CD274 (PD-L1) BV421 and CD19 APC-H7 (both Becton Dickinson). BM or PB immune cell subsets were identified and analysed for cell surface marker expression levels by staining 1.0×10⁶ cells/mL with CD45 Krome Orange, CD56 PC7 (both Beckman Coulter), CD14 APC-H7, CD19 APC-H7, CD3 V450, CD4 APC-H7 or PE, CD8 FITC, CD45-RA APC. CD127 PE.Cy7, CD62L PE, CD274 (PD-1) BV421, CD16 APC, HLA-DR APC-H7 (all Becton Dickinson) and CD25 PE (Dako) or with CD4 BUV395 (BD Biosciences), CD8 BUV737 (BD

Biosciences), PD-1 BV421 (BD Biosciences), TIM-3 BV650 (BD Biosciences), CD3 BV711 (BD Biosciences), CD45RO BV786 (BD Biosciences). CD38 Humab-003-FITC (Janssen). CD45RA PerCP-Cy5.5 (BD Biosciences). HLA-DR PE (BD Biosciences), LAG-3 PE-eF610 (ThermoFisher), CD25 PE-Cy7 (BD Biosciences), CCR7 AF647 (BD Biosciences), CD127 APC-eF780 (ThermoFisher). All BM samples were analysed within 24 hours from the time the sample was collected.

Flow cytometry was performed using a 7-laser LSRFORTESSA (Becton Dickinson). Fluorescent labeled beads (CS&T beads, Becton Dickinson) were used daily to monitor the performance of the flow cytometer and verify optical path and stream flow. This procedure enables controlled standardized results and allows the determination of long-term drifts and incidental changes within the flow cytometer. No changes were observed which could affect the results. Compensation beads were used to determine spectral overlap, and compensation was automatically calculated using Diva software. Flow cytometry data were analyzed using FACS Diva software.

### Cytogenetic Analysis

Cytogenetic abnormalities were assessed in purified MM cells by fluorescence in situ hybridization (FISH) and single nucleotide polymorphism (SNP) array. High-risk disease was defined by the presence of del(17p), del(1p), ampl(1q), t(4;14) or t(14;16)².

### Multiplex Cytokine Assay

Cytokines [interferon-gamma (IFN-γ), interleukin (IL)-2, IL-6, IL-8, IL-10, and tumor necrosis factor-alpha (TNF-α)] in the cell culture supernatants were analyzed using V-Plex proinflammatory Panel 1 Human Kit (Meso Scale Diagnostics), according to the manufacturer’s protocol.

### Statistics

Comparisons between variables were performed using two-tailed (paired) Student’s t-test, or Mann-Whitney U test or Wilcoxon matched-pairs signed-rank test in case the data do not follow a normal distribution. Correlations between variables were made using the Spearman’s rank correlation coefficient. P-values below 0.05 were considered significant. In case of combinatorial treatment of JNJ-564 and daratumumab, the expected lysis values were calculated to test the null hypothesis that there is only an additive effect between JNJ-564 and daratumumab, using the following formula: % expected lysis = (% lysis with JNJ-564 + % lysis with daratumumab) - (% lysis with JNJ-564 x % lysis with daratumumab). The null hypothesis of “additive effects” was rejected, if the observed values were significantly higher (P<0.05) than the expected values.

### Example 1 Phase 1 Study of Talquetamab (JNJ-564) Administered in Combination with Subcutaneous Daratumumab for Relapsed or Refractory Multiple Myeloma (RRMM) (TRIMM-2)

A phase 1b, open-label, multicenter, multi-cohort study of talquetamab in combination with subcutaneous (SC) dosing regimens of daratumumab administered to adult subjects with multiple myeloma was carried out. Two treatment combinations also included pomalidomide (and concomitant dexamethasone for at least the initial cycles). In the pomalidomide-containing treatment combinations, dexamethasone administration was required through the first 3 full immunomodulatory agent (IMiD)containing cycles to enhance IMiD-driven antimyeloma effects and serve as pretreatment medication for daratumumab and talquetamab. An overall aim of the study was to evaluate the safety of daratumumab in combination with talquetamab with or without pomalidomide (and concomitant dexamethasone at least in the initial cycles), and to evaluate preliminary antitumor activity. Safety was monitored by a Study Evaluation Team (SET).

### Objectives and Endpoints

TABLE 10

Objectives and endpoints of the phase 1 study of talquetamab (JNJ-564) administered in combination with subcutaneous daratumumab for RRMM

| Objectives | Endpoints |
|---|---|
| Primary | |
| Part 1: To identify RP2Ds for each treatment combination | Frequency and severity of dose-limiting toxicities |
| Part 2: To characterize the safety of each RP2D for selected treatment combination(s) | Frequency and severity of adverse events and serious adverse events |
| Secondary | |
| • To characterize the pharmacokinetics and pharmacodynamics of each study treatment | • Serum concentrations and pharmacodynamic markers |
| • To assess the immunogenicity of each study treatment | • Presence of anti-drug antibodies |
| • To evaluate the antitumor activity of eachtreatment comzbination | • ORR |
| | • Clinical benefit rate (MR or better) |
| | • Duration of and time to response |

Exploratory
• To explore relationships between pharmacokinetics, pharmacodynamics, adverse event profile, and clinical activity
• To investigate predictive biomarkers of response or resistance
• To investigate the immunoregulatory activity of each treatment combination
• To evaluate MRD negativity rates
• To evaluate PFS
• To evaluate the exposure-response relationship
Abbreviations: MR=minimal response; MRD=minimal residual disease; ORR=overall response rate; PFS=progression-free survival; P2D=recommended Phase 2 dose

### Study Design

The study was conducted in 2 parts:
Part 1: dose escalation to establish the RP2D(s) of each treatment combination
Part 2: dose expansion at the RP2D(s) for selected treatment combination(s).

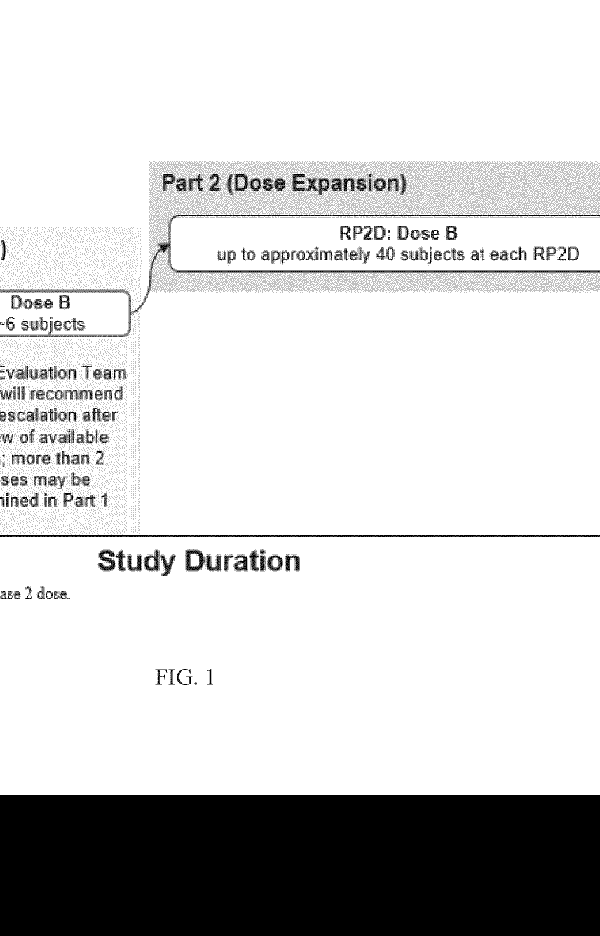

A schematic overview of Part 1 and Part 2 is provided in FIG. 1. The following treatment combinations were studied:
SC daratumumab and IV talquetamab
SC daratumumab and SC talquetamab
SC daratumumab, SC talquetamab, and pomalidomide.

### Part 1 (Dose Escalation Part)

In Part 1 (dose escalation), all subjects would receive daratumumab at the approved dose in multiple myeloma. Subjects who receive pomalidomide would receive it at its approved dose or at a lower modified dose, as applicable. Dosing Schedule B was approved for use in the study, in which step-up dosing begins on Cycle 1 Day 2. For subsequent cohorts, the SET would determine the step-up dosing regimen and treatment dose based on a statistical model using all available safety, pharmacokinetic, and pharmacodynamic data to identify safe and tolerable RP2D(s). Step-up and treatment doses examined in this study would not exceed those previously cleared by the SET in the monotherapy studies of talquetamab. The relevant treatment dose was planned to be administered on a weekly basis in 28-day cycles following the step-up dose(s); however, other schedules, including biweekly administration, were studied in cohorts as well. At least 30 subjects would be evaluated in Part 1. The total number of subjects enrolled would depend on the number of dose levels explored to identify the RP2D(s) and the number of subjects enrolled at each dose level.

### Part 2 (Dose Expansion Part)

The RP2D(s) for each treatment combination selected for further study in Part 2 (NCT04108195CTX) would be based on the dose recommended by BLRM (Bayesian Logistic Regression Model) based on the findings from Part 1. Additionally, the SET would review all available safety, pharmacokinetic, pharmacodynamic, and efficacy data for each treatment combination in Part 1 before determining the RP2D(s) for that treatment combination in Part 2, if applicable. The SET may select 1 or more RP2D(s) for each treatment combination. Up to approximately 40 subjects would be evaluated in each of the RP2Ds for each treatment combination selected for study in Part 2.

### Subject Population

The study was conducted on subjects ≥18 years of age with multiple myeloma who had received at least 3 prior lines of therapy, including a proteasome inhibitor (PI) and an IMiD, or who have disease that is double refractory to a PI and an IMiD. Subjects who had received anti-CD38 therapy for ≤ 90 days were excluded. For subjects who were to be enrolled in a treatment combination that includes pomalidomide, prior IMiD therapy should include lenalidomide.

The inclusion and exclusion criteria for enrolling subjects in this study are described below. All study enrollment criteria have been met at screening and prior to first dose of study drug.

### Inclusion Criteria

Each subject was required to satisfy all of the following criteria for enrollment in the study:

1. ≥18 years of age.

2. Documented initial diagnosis of multiple myeloma according to IMWG diagnostic criteria.

3. Must have either of the following:
Received at least 3 prior lines of therapy (see definition below) including a PI (≥2 cycles or 2 months of treatment) and an IMiD (≥2 cycles or 2 months of treatment) in any order during the treatment (except for subjects who discontinued either of these treatments due to a severe allergic reaction within the first 2 cycles/months).
   Undergone at least 1 complete cycle of treatment for each line of therapy, unless progressive disease was the best response to the line of therapy.
   For prior lines of therapy without a PI and/or IMiD, to meet the criteria for at least three prior lines of therapy the subject must have undergone at least 1 complete cycle or month of treatment, unless progressive disease was the best response to the line of therapy, or unless the subject discontinued due to an adverse reaction: or
Disease that is double refractory to a PI and an IMiD. For subjects who have received more than 1 type of PI, the disease must be refractory to the most recent one. Similarly, for those who have received more than 1 type of IMiD, the disease must be refractory to the most recent one.

Note: Subject must have documented evidence of progressive disease based on investigator’s determination of response by the IMWG 2016 criteria as described by Kumar et al. 2016 (Lancet Oncol. 2016:17(8):e328-346.) on or within 12 months of their last line of therapy. Confirmation may be from either central or local testing. Also, subjects with documented evidence of progressive disease within the previous 6 months and who are refractory or non-responsive to their most recent line of therapy afterwards are eligible. For subjects who are to be enrolled in a treatment combination that includes pomalidomide, prior IMiD therapy should include lenalidomide. A single line of therapy may consist of 1 or more agents, and may include induction, hematopoietic stem cell transplantation, and maintenance therapy. Specifically, a line of therapy consists of ≥1 complete cycle of a single agent, a regimen consisting of a combination of several drugs, or a planned sequential therapy of various regimens (e.g.. 3 to 6 cycles of initial therapy with bortezomib-dexamethasone followed by stem cell transplantation, consolidation, and lenalidomide maintenance is considered 1 line). Radiotherapy, bisphosphonate, or a single short course of steroids (i.e., less than or equal to the equivalent of dexamethasone 40 mg/day for 4 days) would not be considered prior lines of therapy.

4. Measurable disease at screening as defined by any of the following:
Serum monoclonal protein (M-protein) level ≥1.0 g/dL (in non-IgG myeloma, an M-protein level ≥0.5 g/dL); or
Urine M-protein level ≥200 mg/24 hours; or
Light chain multiple myeloma: Serum Ig free light chain (FLC) ≥10 mg/dL and abnormal serum Ig kappa lambda FLC ratio.

5. Eastern Cooperative Oncology Group (ECOG) performance status grade of 0 or 1 at screening and at Cycle 1, Day 1 predose.

6. Clinical laboratory values meeting the following criteria prior to administration of daratumumab on Cycle 1 Day 1:

TABLE 11

Criteria for clinical laboratory values
Hematology

| Hemoglobin | ≥8.0 g/dL (≥5 mmol/L) (without RBC transfusion in the prior 7 days; recombinant human erythropoietin use is permitted) |
|---|---|
| Platelets | ≥50× 10⁹/L (without transfusion support in the prior 7 days) |
| Absolute Neutrophil Count (ANC) | ≥1.0× 10⁹/L (prior growth factor support is permitted but must be without support for 7 days for G-CSF or GM-CSF or 14 days for pegvlated-G-CSF) |
| Chemistry | |
| Aspartate aminotransferase (AST) and alanine aminotransferase (ALT) | ≤2.5×ULN |
| Creatinine clearance | ≥30 mL/min/1.73 m² based upon Modified Diet in Renal Disease formula calculation |
| Total bilirubin | ≤1.5 × ULN; except in subjects with congenital bilirubinemia, such as Gilbert syndrome (in which case direct bilirubin ≤1.5×ULN is required) |
| Serum calcium corrected for albumin | ≤14 mg/dL (≤3.5 mmol/L) or free ionized calcium <6.5 mg/dL (<1.6 mmol/L) |

1. Abbreviations: G-CSF=granulocyte colony-stimulating factor; GM-CSF=granulocyte-macrophage colony-stimulating factor, ULN=upper level of normal; RBC=red blood cell

7. Women of childbearing potential must have a negative highly-sensitive serum β human chorionic gonadotropin (β-hCG) pregnancy test (<5 IU/mL) at screening and a negative urine or serum pregnancy test within 1 day before the first dose of study drug and must agree to further serum or urine pregnancy tests during the study.

8. Women must be either of the following:
a. Not of childbearing potential
b. Of childbearing potential and
   practicing true abstinence;
   or have a sole partner who is vasectomized;
   or practicing at least 1 highly effective user independent method of contraception (e.g., intrauterine device (IUD), intrauterine hormone-releasing system (IUS), bilateral tubal ligation/occlusion, or implantable progestogen-only hormone contraception associated with inhibition of ovulation). If hormonal contraception is used (e.g., oral estrogen/progestin), a male or female condom with or without spermicide (e.g. spermicidal foam/gel/film/cream/suppository) must also be used.
   For subjects receiving pomalidomide, women of childbearing potential must be on 2 methods of reliable birth control simultaneously while receiving study treatment and until 100 days after last dose of study treatment: one highlight effective form of contraception (tubal ligation, intrauterine device, hormonal [oral, injectable, transdermal patches, vaginal rings, or implants], or partner’s vasectomy), and 1 additional effective contraceptive method (male latex or synthetic condom, diaphragm, or cervical cap).
   For subjects not receiving pomalidomide, a women of childbearing potential using oral contraceptives must use an additional contraceptive method.
Subject must agree to continue the above while receiving study drug and until 100 days after last dose. Women of childbearing potential must agree to pregnancy testing (serum or urine) within 100 days after the last study drug administration.

Note: If a woman becomes of childbearing potential after start of the study the woman must comply with point (b.) as described above.

A woman using oral contraceptives must use an additional contraceptive method in addition to the requirements listed above.

9. Men must wear a condom (with or without spermicidal foam/gel/film/cream/suppository) when engaging in any activity that allows for passage of ejaculate to another person, during the study and for 100 days after the last dose of study drug. His female partner, if of childbearing potential, must also be practicing a highly effective method of contraception (e.g., intrauterine device (IUD), intrauterine hormone-releasing system (IUS), combined (estrogen- and progestogen-containing) hormonal contraception associated with inhibition of ovulation, etc.).

If the male subject is vasectomized, he still must wear a condom (with or without spermicidal foam/gel/film/cream/suppository), but his female partner is not required to use contraception.

10. Women must agree not to donate eggs (ova, oocytes) or freeze for future use, for the purposes of assisted reproduction during the study and for at least 100 days after the last dose of study drug.

11. Men must agree not to donate sperm for the purpose of reproduction during the study and for at least 100 days after receiving the last dose of study drug.

12. Sign an informed consent form (ICF) indicating that he or she understands the purpose of and procedures required for the study and is willing to and able to participate in the study. Consent is to be obtained prior to the initiation of any study related tests or procedures that are not part of standard of care for the subject’s disease.

13. Willing and able to adhere to the prohibitions and restrictions specified in this protocol.

### Exclusion Criteria

Any potential subject who meets any of the following criteria will be excluded from participating in the study:

1. Treatment in the prior 90 days with an anti-CD38 therapy (e.g., daratumumab), or discontinuation of a prior anti-CD38 therapy at any time due to an adverse event related to the anti-CD38 therapy.

2. Prior antitumor therapy as follows, before the first dose of study drug:
Targeted therapy, epigenetic therapy, or treatment with an investigational drug or an invasive medical device within 21 days or at least 5 half-lives, whichever is less.
Monoclonal antibody treatment within 21 days (anti-CD38 treatment cannot be used within the prior 90 days).
Cytotoxic therapy within 21 days.
PI therapy within 14 days.
IMiD therapy within 7 days.
Radiotherapy within 21 days. However, if the radiation portal covered ≤5% of the bone marrow reserve, the subject is eligible irrespective of the end date of radiotherapy.
Gene modified adoptive cell therapy (e.g., chimeric antigen receptor modified T cells, NK cells) within 90 days.

3. A cumulative dose of corticosteroids equivalent to ≥140 mg of prednisone within the 14-day period before the first dose of study drugs.

4. Live, attenuated vaccine within 4 weeks prior to the first dose of study drug unless approved by sponsor.

5. Toxicity from previous anticancer therapy that has not resolved to baseline levels or to Grade ≤1 (except alopecia [any grade] or peripheral neuropathy Grade ≤3).

6. Stem cell transplantation:
Subjects who received an allogeneic transplant must be off all immunosuppressive medications for ≥42 days without signs of graft-versus-host disease
Autologous stem cell transplantation ≤12 weeks before the first dose of study drug

7. Active central nervous system involvement or exhibits clinical signs of meningeal involvement of multiple myeloma. If either is suspected, brain magnetic resonance imaging (MRI) and lumbar cytology are required.

8. Active plasma cell leukemia, Waldenström’s macroglobulinemia, POEMS syndrome (polyneuropathy, organomegaly, endocrinopathy, M-protein, and skin changes), or primary amyloid light chain amyloidosis.

9. Known to be seropositive for human immunodeficiency virus.

10. Seropositive for hepatitis B (defined by a positive test for hepatitis B surface antigen [HBsAg]). Subjects with resolved infection (i.e., subjects who are HBsAg negative with antibodies to total hepatitis B core antigen [Anti-HBc] with or without the presence of hepatitis B surface antibodies [Anti-HBs]) must be screened using real-time polymerase chain reaction (PCR) measurement of HBV DNA levels. Those who are PCR positive will be excluded. EXCEPTION: Subjects with serologic findings suggestive of HBV vaccination (Anti-HBs positivity as the only serologic marker) AND a known history of prior HBV vaccination, do not need to be tested for HBV DNA by PCR.

11. Active hepatitis C infection as measured by positive hepatitis C virus (HCV)-RNA testing. Subjects with a history of Hepatitis C virus antibody positivity must undergo HCV-RNA testing

12. Either of the following:
Chronic obstructive pulmonary disease (COPD) with forced expiratory volume in 1 second (FEV1) <50% of predicted normal. NOTE: FEV1 testing is required for subjects suspected of having COPD and subjects must be excluded if FEV1 is <50% of predicted normal.
Moderate or severe persistent asthma within the past 2 years, or uncontrolled asthma of any classification. NOTE: subjects who currently have controlled intermittent asthma or controlled mild persistent asthma are allowed to participate in the study.

13. Allergies, hypersensitivity, or intolerance to any study intervention or its excipients (refer to Investigator’s Brochures and package inserts).

14. Any serious underlying medical condition, such as:
Evidence of serious active viral, bacterial, or uncontrolled systemic fungal infection
Active autoimmune disease requiring systemic immunosuppressive therapy within 6 months before start of study treatment. EXCEPTION: Participants with vitiligo, type I diabetes, and prior autoimmune thyroiditis that is currently euthyroid based on clinical symptoms and laboratory testing are eligible regardless of when these conditions were diagnosed.
Disabling psychiatric conditions (e.g., alcohol or drug abuse), severe dementia, or altered mental status.
Any other issue that would impair the ability of the subject to receive, absorb, or tolerate the planned treatment at the investigational site, to understand informed consent or any condition for which, in the opinion of the investigator, participation would not be in the best interest of the subject (e.g., compromise the well-being) or that could prevent, limit, or confound the protocol-specified assessments.

15. The following cardiac conditions:
New York Heart Association stage III or IV congestive heart failure
Myocardial infarction or unstable angina ≤6 months prior to enrollment
History of clinically significant ventricular arrhythmia or unexplained syncope, not believed to be vasovagal in nature or due to dehydration
History of severe non-ischemic cardiomyopathy
Screening 12-lead electrocardiogram (ECG) showing an average baseline QT interval as corrected by Fridericia’s formula (QTc) of >470 msec

16. Pregnant or breastfeeding or planning to become pregnant while enrolled in this study or within 100 days after the last dose of study drug.

17. Plans to father a child while enrolled in this study or within 100 days after the last dose of study drug.

18. Major surgery within 2 weeks of the first dose, will not have fully recovered from surgery, or has surgery planned during the time the subject is expected to be treated in the study.

Note: Subjects with planned surgical procedures to be conducted under local anesthesia may participate.

Any potential subject who meets any of the following criteria will be excluded from participating in a treatment combination containing pomalidomide:

19. Subject previously experienced an adverse event related to pomalidomide that required discontinuation of treatment.

### Study Interventions

The treatments were administered in 28-day cycles. Daratumumab was administered to all subjects by SC injection at a dose of 1800 mg as follows: weekly in Cycles 1-2, every 2 weeks (Q2W) in Cycles 3-6, and every 4 weeks thereafter. Note that a recombinant human hyaluronidase PH20 (rHuPH20) was used to decrease the injection volume required, facilitating the SC administration of daratumumab.

Daratumumab SC (Dara) was administered in combination with different dosage levels of talquetamab (Tal), including Dara 1800 mg + Tal 60 µg/kg SC weekly, Dara 1800 mg + Tal 400 µg/kg SC weekly. Dara 1800 mg + Tal 400 µg/kg SC biweekly starting Cycle 3 Day 1 (Tal 400 µg/kg SC weekly in Cycles 1-2), and Dara 1800 mg + Tal 800 µg/kg SC biweekly. Some subjects in the Dara 1800 mg + Tal 400 µg/kg SC weekly cohort switched to Tal 800 µg/kg biweekly SC dosing after Cycle 3 Day 1. Except for the 60 µg/kg talquetamab SC weekly cohort in which step-up dosing began on Cycle 1 Day 9, step-up doses for talquetamab began on Cycle 1 Day 2. When daratumumab and talquetamab were administered on the same day, daratumumab was administered first. Step-up Dose 1 of talquetamab was administered at least 20 hours after SC daratumumab. Subsequent step-up dose(s), if applicable, and first treatment dose of talquetamab were administered approximately 3 hours after SC daratumumab. Subsequent treatment doses of talquetamab was administered approximately 1 hour after SC daratumumab (when both study drugs are administered on the same day).

For treatments involving a combination of daratumumab SC, talquetamab SC and pomalidomide, pomalidomide was orally self-administered once per day at 2 mg, 4 mg, or a combination thereof. Pomalidomide cohorts included 2 mg starting at C2D1, 4 mg starting at treatment onset, and 2 mg starting at C2D1 elevating to 4 mg starting at C4D1. Pomalidomide can be taken before or after study drugs in the treatment combination. To minimize the potential for increased risk of cytokine release syndrome (CRS) with concurrent administration of a bispecific antibody and pomalidomide, the initial cohort of subjects would receive a delayed dosing schedule of pomalidomide. If deemed appropriate by the SET, a decreased starting dose or later start date (e.g., Cycle 2 Day 1 start) could be implemented for pomalidomide for future cohorts, based on a review of safety data for the regimen. Dexamethasone will be given concurrently with the first 3 full IMiD-containing cycles. During the first week of Cycle 1, dexamethasone 20 mg would be given on Cycle 1 Day 1 prior to daratumumab SC and 2 additional doses of dexamethasone 16 mg would be given, 1 each prior to Step-up Dose 1 and Step-up Dose 2 of the bispecific antibody. For the remainder of Cycle 1 and subsequent required cycles, dexamethasone would be given at 40 mg (oral or IV) weekly (except for subjects >75 years of age or who have body mass index [BMI] <18.5, who should receive 20 mg of dexamethasone prior to daratumumab SC administration only). Dexamethasone is given approximately 1 to 3 hours prior to daratumumab SC (or the bispecific antibody on days on which daratumumab SC is not administered). After the required dexamethasone cycles indicated above, the continuation of and the administration schedule for dexamethasone to enhance IMiD-driven antimyeloma effects would be based on the clinical judgment of the investigator. If pomalidomide is permanently discontinued due to toxicity or intolerance, then high dose dexamethasone may also be discontinued based on the clinical judgment of the investigator.

To minimize the potential for increased risk of cytokine release syndrome (CRS) with concurrent administration of a bispecific antibody and pomalidomide, the initial cohort of subjects received a delayed dosing schedule of pomalidomide (C2D1 (cycle 2, day 1) or C1D15 (cycle 1, day 15)). The first subject in each cohort in Part 1 was observed for at least 36 hours after the first administration of talquetamab or before treating subsequent subjects at the treatment dose.

The treatment also includes required and optional pretreatment and posttreatment medication associated with SC daratumumab and pomalidomide. The required and optional pretreatment and posttreatment medications for daratumumab can be 2-week Glucocorticoid Taper. It can be, for example, the required treatment with IV or oral glucocorticoid (e.g., methylprednisolone 20 to 100 mg, dexamethasone 4 to 12 mg) before and after daratumumab administration for subjects not receiving pomalidomide; the required IV or oral antihistamine (e.g., diphenhydramine 25 to 50 mg or equivalent) or antipyretic (acetaminophen 650 to 1000 mg) before daratumumab administration for all subjects; and the optional IV or oral glucocorticoid (methylprednisolone 60 mg (or dexamethasone 12 mg), or oral leukotriene inhibitor (e.g., montelukast 10 mg) before daratumumab administration for all subjects. IV or oral glucocorticoid (e.g., dexamethasone, 8 to 16 mg), antihistamine (e.g., diphenhydramine 25 to 50 mg or equivalent) or antipyretic (acetaminophen 650 to 1000 mg) could also be required as pretreatments for talquetamab, e.g., before all step-up doses and first treatment dose, or for subjects who experience Grade ≥2 CRS/IRR for the next 2 subsequent doses of talquetamab.

For subjects in any treatment combination with a higher risk of respiratory complications (e.g., subjects with mild asthma or subjects with COPD who have an FEV1 <80% at screening or developed FEV1 <80% during the study without any medical history), the following postinjection medications should be considered: antihistamine, short-acting β2 adrenergic receptor agonist such as salbutamol, control medications for lung disease (e.g., inhaled corticosteroids ± long-acting β2 adrenergic receptor agonists for subjects with asthma; long-acting bronchodilators such as tiotropium or salmeterol ± inhaled corticosteroids for subjects with COPD).

### Study Assessments

Safety, pharmacokinetics, immunogenicity, biomarkers, efficacy, and other measurements would be performed.

Safety would be assessed by, e.g., physical examinations (including neurological assessment), Eastern Cooperative Oncology Group (ECOG) performance status, clinical laboratory tests, vital signs, adverse event monitoring, and concomitant medication usage. All adverse events and special reporting situations, whether serious or nonserious, will be reported from the time a signed and dated ICF is obtained until 100 days after the last dose of study drug or until the start of subsequent systemic anticancer therapy, if earlier, and may include contact for follow-up of safety. Adverse events (AEs) are assessed by NCI-CTCAE v5.0, except for cytokine release syndrome (CRS) and immune effector cell-associated neurotoxicity syndrome (ICANS), which were graded per American Society for Transplantation and Cellular Therapy (ASTCT) guidelines. Events of CRS (any grade) must be followed until recovery or until there is no further improvement.

Blood and serum or plasma samples were collected for assessments of pharmacokinetics and immunogenicity (e.g., antibodies to daratumumab, rHuPH20, or talquetamab). Selection of the dose regimen (dose level and frequency) for dose expansion was determined based on the pharmacokinetic and pharmacodynamic information obtained during dose escalation. A sample for the pharmacokinetic and immunogenicity analysis was collected at scheduled time and any time a suspected IRR or CRS event (in case of a CRS event, samples were collected at onset, 24 hours and 72 hours) was observed during the study.

Each serum sample was evenly divided into 3 aliquots (1 for pharmacokinetics and immunogenicity of daratumumab, 1 for pharmacokinetics and immunogenicity of talquetamab, and 1 backup). Each plasma sample for anti-rHuPH20 antibodies were divided into 5 aliquots (3 for anti-rHuPH20 antibodies and 2 for neutralizing antibodies against rHuPH20). Samples collected for analyses of pharmacokinetics and immunogenicity can be used to evaluate a marker of disease, such as sBCMA, or to evaluate safety or efficacy aspects that address concerns arising during or after the study period for further characterization of immunogenicity. For the pharmacokinetics analysis, serum samples were analyzed to determine concentrations of daratumumab and talquetamab using validated, specific, and sensitive assay methods. Pharmacokinetic parameters include, but are not limited to, area under the curve (AUC)₍₀₋ₜ₎, AUCₜₐᵤ, Cₘₐₓ, and Tₘₐₓ would be calculated if sufficient data were available for estimation. For immunogenicity analysis, the detection and characterization of antibodies to daratumumab, rHuPH20, and talquetamab were performed using validated assay methods. Positive samples for binding antibodies were tested for neutralizing antibodies to daratumumab or talquetamab. For the rHuPH20 immunogenicity assessments, plasma samples were screened for antibodies binding to rHuPH20 and were assessed in confirmatory and titer assays as necessary.

Biomarker assessments were conducted in both Part 1 and Part 2. The biomarker assessments focused on several main objectives: (1) immune responses indicative of T cell redirection for potential contributions to response to study drug; (2) the ability of each treatment combination to induce MRD negativity in subjects with multiple myeloma who have achieved a CR: (3) serum proteomic profiling of cytokines (such as IL-6, IL-2, and IL-10) or other serum proteins indicative of immune response; (4) biomarkers of response/resistance on myeloma cells (such as GPRC5D and PD-L1); (5) the clinical benefit (ORR, duration of response [DOR], and time to response) of each treatment combination in subjects with cytogenetic modifications (del17p, t(4;14), t(14;16), or other high-risk molecular subtypes); and (6) immunophenotypes of immune cells subsets such as CD4+ and CD8+ T cells, and regulatory T cells that could directly impact the mechanisms of action. Additional biomarker samples could be collected to help understand an unexplained adverse event. Additional sample(s) for cytokines could also be collected any time a suspected IRR or CRS event was observed or reported during the study.

Disease evaluations would be performed by a central laboratory (additional samples may be collected for analysis by the local laboratory) until disease progression. This study used the IMWG-based response criteria (2016) as described by Kumar et al. (Lancet Oncol. 2016:17(8):e328-346.) For subjects with suspected daratumumab interference on serum immunofixation electrophoresis (IFE), a second reflex assay using the anti-idiotype monoclonal antibody was used to confirm daratumumab migration on the IFE. Subjects that meet all other IMWG criteria for CR, and whose positive IFE is confirmed to be daratumumab interference, are considered complete responders. For subjects with light chain multiple myeloma, both serum and urine IFE and serum FLC assay were performed every 4 weeks. Additional serum samples may be utilized to monitor for potential daratumumab interference with the IFE and response adjudicated per IMWG-based response criteria. Quantitative immunoglobulin (QIg, e.g., IgG, IgA, IgM, IgE, and IgD), M-protein by electrophoresis (SPEP), FLC and IFE measurements in serum and urine and serum β-microglobulin were analyzed by the central laboratory. Disease progression based on one of the laboratory tests alone must be confirmed by at least 1 repeat investigation performed 1 to 3 weeks later. Disease evaluations would continue beyond relapse from CR until disease progression was confirmed. Serum and urine IFE and serum FLC assays would be performed at screening and thereafter when a CR or sCR is suspected (when serum or 24-hour urine M-protein electrophoresis [by SPEP or UPEP] are 0 or non-quantifiable). Development of hypercalcemia (corrected serum calcium >11 mg/dL) may indicate disease progression or relapse if it is not attributable to any other cause. Thus, corrected serum calcium or free ionized calcium was also analyzed in blood samples until the development of confirmed disease progression.

Bone marrow aspirate or biopsy would be performed for clinical assessments and biomarker evaluations. Clinical staging (morphology, cytogenetics, and immunohistochemistry or immunofluorescence or flow cytometry) may be done by a local laboratory. A portion of the bone marrow aspirate was used for immunophenotyping and to monitor GPRC5D, CD38, and checkpoint ligand expression in CD138-positive multiple myeloma cells, and checkpoint expression on T cells. A bone marrow aspirate sample was required to confirm CR and sCR before the next scheduled dose of study drug. MRD negativity is being evaluated in the field as a potential surrogate for progression-free survival (PFS) and OS. Baseline bone marrow aspirates was used to define the myeloma clones, and posttreatment samples will be used evaluate MRD negativity in those subjects who experience a CR/sCR. Bone marrow aspirate DNA can be used to monitor MRD using next generation sequencing, while serum MRD negativity will be assessed via mass spectrometry.

A complete skeletal survey (including skull, entire vertebral column, pelvis, chest, humeri, femora, and any other bones for which the investigator suspects involvement by disease) was performed during the screening period and evaluated by either roentgenography or low-dose computed tomography (CT) scans (or positron emission tomography [PET]/CT) without the use of IV contrast. The same methodology that was used at baseline should be used when assessing for progression during the treatment period. MRI may also be included for evaluation of bone disease.

### Statistical Analysis

No formal statistical hypothesis testing was conducted in this study. Part 1 (dose escalation) was supported by a modified continual reassessment method (mCRM) based on a statistical model, Bayesian Logistic Regression Model (BLRM), with Escalation with Overdose Control (EWOC) principle. One or more RP2D(s) for each treatment combination may be identified. In Part 2 (dose expansion), subjects were treated at each RP2D(s) to further assess the safety and antitumor activity of selected treatment combination(s).

### Endpoint Definitions

ORR is defined as the proportion of subjects who have a PR or better according to the IMWG criteria. Response to treatment will be evaluated by investigator.
Clinical benefit rate (ORR + MR) is defined as the proportion of subjects who have a MR or better according to the IMWG criteria, as evaluated by investigator.
MRD negativity rate is defined as the proportion of subjects who achieve MRD negative status.
DOR is defined as the time from the date of initial documentation of a response (PR or better) to the date of first documented evidence of progressive disease, as defined in the IMWG criteria or death due to progressive disease, whichever occurs first. Relapse from CR is not considered as disease progression. For subjects who have not progressed, data will be censored at the last disease evaluation before the start of any subsequent antimyeloma therapy.
Time to response is defined as the time between date of first dose of study drug and the first efficacy evaluation that the subject has met all criteria for PR or better.
PFS is defined as the time from the date of first dose of study drug to the date of first documented disease progression, as defined in the IMWG criteria, or death due to any cause, whichever occurs first. For subjects who have not progressed and are alive, data will be censored at the last disease evaluation before the start of any subsequent antimyeloma therapy.

### Preliminary Results (Cut-off Date for Analyses Apr. 6, 2022)

The study is ongoing and as of the cut-off date for the analysis, 129 patients treated with talquetamab and daratumumab +/- pomalidomide were evaluated.

### Talquetamab SC + Daratumumab SC Cohorts

SC treatment with daratumumab and talquetamab was administered in 28-day cycles (with step-up dosing for talquetamab). Data were pooled into daratumumab 1800 mg plus talquetamab (400 µg/kg weekly + 400 µg/kg or 800 µg/kg biweekly +/pomalidomide).

Preliminary data as of Apr. 6, 2022 included results for 14 participants treated with SC talquetamab treatment doses of 400 µg/kg weekly, in combination with daratumumab 1800 mg SC. Five participants were treated with SC talquetamab treatment doses of 400 µg/kg every other week starting on C2D1 (Cycle 2 Day 1), in combination with daratumumab 1800 mg SC. An additional cohort of 44 participants received SC talquetamab treatment doses of 800 µg/kg every other week starting on C1D15 (Cycle 1 Day 15), in combination with daratumumab 1800 mg SC. For the cohorts that included pomalidomide, participants received SC talquetamab 400 µg/kg weekly (n=26) or 800 µg/kg every other week (n=32) in combination with daratumumab 1800 mg SC and 2 mg pomalidomide starting C2D1 (Cycle 2 Day 1; n=18) or 4 mg pomalidomide starting C1D15 (Cycle 1 Day 15; n=8). In addition, 8 participants received SC talquetamab 800 µg/kg every other week in combination with daratumumab 1800 mg SC and 2 mg pomalidomide starting C2D1 (Cycle 2 Day 1) with a planned dose increase of pomalidomide 4 mg C4D1 (Cycle 4 Day 1). All participants received talquetamab in combination with 1800 mg daratumumab SC. Dexamethasone was given as pretreatment medication during step-up dosing and for the first treatment dose of talquetamab SC, as well as for the first 2 doses of daratumumab SC. after which time dexamethasone was discontinued. A summary of the talquetamab and daratumumab dosing cohorts is shown in Table 12.

TABLE 12

Talquetamab and daratumumab dosing cohorts (n=129)

| Talquetamab^{a} | Daratumumab | Number of Patients |
|---|---|---|
| 400 µg/kg SC QW | 1800 mg SC Cycles 1-2: QW Cycles 3-6: Q2W Cycles 7+: monthly | 14 |
| 400 µg/kg SC Q2W | | 5 |
| 800 µg/kg SC Q2W | | 44 |
| 400 µg/kg SC QW + 2 mg Pom C2D1 or 4 mg Pom on a delayed schedule | | 26 |
| 800 µg/kg SC Q2W + 2 mg pom C2D1 | | 32 |
| 800 µg/kg SC Q2W + 2 mg pom C2D1, 4 mg C4D1 | | 8 |

SC= subcutaneous, QW =once weekly, Q2W= once every 2 weeks, pom= pomalidomide; C2D1 = Cycle 2, Day 1; C4D1 = Cycle 4 Day 1 a. 1-3 step-up doses were given within 1 week before a full dose. Note that premedications (e.g., glucocorticoid, antihistamine, and antipyretic) were limited to step-up doses and first full dose (no steroid requirement after first full dose).

Median age range for the 129 subjects evaluated was 63 years (range 33-81) and 58 subjects were female (45.0%). Median number of prior therapies was 5 (range 2-18), 79.1% of subjects were refractory to last line of therapy, 65.1% triple-class refractory, 67.4% penta-class exposed, and 33.3% penta-class refractory. A summary of the subjects’ demographics and baseline characteristics is shown in Table 13.

TABLE 13

Patient demographics and baseline characteristics

| Characteristic | Dara + Tal^{a} (n=63) | Dara + Tal + Pom^{b} (n=66) | Dara + Tal (n=129) |
|---|---|---|---|
| Age, median (range), years | 64 (44-81) | 63 (33-81) | 63 (33-81) |
| Female, n % | 33 (52.4) | 25 (37.9) | 58 (45.0) |
| Race, n (%) | | | |
| White | 49 (77.8) | 49 (74.2) | 98 (76.0) |
| Black/African American | 9 (14.3) | 7 (10.6) | 16 (12.4) |
| Not Reported | 5 (7.9) | 9 (13.6) | 14 (12.9) |
| Time since diagnosis, years, median (range) | 6.53 (1.6-18.0) | 6.78 (0.3-18.3) | 6.6 (0.3-18.3) |
| Baseline ISS Stage ^{c}, n (%) | | | |
| I | 23 (46.0) | 34 (56.7) | 57 (51.8) |
| II | 19 (38.0) | 14 (23.3) | 33 (30.000) |
| III | 8 (16.0) | 12 (20.0) | 20 (18.2) |
| High cytogenetic risk ^{d}, n (%) | 8 (19.5) | 16 (27.1) | 24 (24.0) |
| Extramedullary plasmacytomas ≥ 1^{e}, n (%) | 15 (23.8) | 12 (18.2) | 27 (20.9) |
| Prior lines of therapy, n, median (range) | 5 (2-18) | 6 (3-17) | 5 (2-18) |
| Prior transplantation | 49 (77.8) | 52 (78.8) | 101 (78.3) |
| Exposure status, n (%) | | | |
| Anti-CD38^{f} | 53 (84.1) | 58 (87.9) | 111 (86.0) |
| IMiD^{g} | 62 (98.4) | 65 (98.5) | 127 (98.4) |
| Prior BCMA therapy ^{h} | 31 (49.2) | 40 (60.6) | 71 (55.0) |
| Triple-class ⁱ | 52 (82.5) | 58 (87.9) | 110 (85.3) |
| Penta-drug^{j} | 41 (65.1) | 46 (69.7) | 87 (67.4) |
| Refractory status, n (%) | | | |
| Anti-CD38 ^{f} | 48 (76.002) | 48 (72.7) | 96 (74.4) |
| IMiD^{g} | 58 (92.1) | 58 (87.9) | 116 (89.9) |
| Triple-class¹ | 40 (63.5) | 44 (66.7) | 84 (65.1) |
| Penta-drug¹ | 20 (31.7) | 23 (34.8) | 43 (33.3) |
| To last time of therapy | 46 (73.0) | 56 (84.8) | 102 (79.1) |

^{a}Dara 1800 mg plus Tal (400 µg/kg SC QW or 400 µg/kg SC Q2W or 800 µg/kg SC Q2W);
^{b}Dara 1800 mg plus Tal (400 µg/kg SC or 800 µg/kg SC) plus pomalidomide
^{c}Percentages calculated from n=50 for Dara + Tal, n=60 for Dara + Tal + Pom, n= 110 for total;
^{d}del(17p), t(4:14), and/or t(14;16); percentages calculated from n=41 for Dara + Tal, n=50 for Dara + Tal + Pom, n=100 for total;
^{e}Soft tissue plasmacytomas not associated with the bone were included;
^{f}Dara or Isa;
^{g}Thalidomide, len, and/or pom;
^{h}BCMA CAR-T therapy or BCMA non-CAR-T therapy;
ⁱ≥1 PI, ≥1 IMiD. and 1 anti-CD38 mAb;
^{j}≥2 PI, ≥2 IMiD, and 1 anti-CD38 mAb.
BCMA, B-cell maturation antigen; CAR-T, chimeric antigen T cell; Dara, daratumumab; IMiD, immunomodulatory drug; Isa, isatuximab; ISS, International Staging System: Len, lenalidomide; mAb, monoclonal antibody; PI, proteasome inhibitor; Pom, pomalidomide: Q2W, every other week; QW, weekly: SC, subcutaneous: Tal, talquetamab.

Among all daratumumab plus talquetamab cohorts (1800 mg daratumumab plus talquetamab (400 µg/kg SC QW or 400 µg/kg SC Q2W or 800 µg/kg SC Q2W)), the combination therapy was tolerable and exhibited a comparable safety profile to both monotherapies. No new toxicities were identified and the majority of adverse events (AEs) were grade 1 or 2. There were 62 patients (98.4%) who experienced AEs and 49 patients (77.8%) experienced grade 3 or 4 AEs. Infections were reported in 34 (54%) patients (grade ≥3: 19%). Skin-related AEs (e.g., SOC for “skin and subcutaneous disorders” with nail disorder, nail ridging, onychomadesis, onychomadesis, and nail dystrophy excluded) occurred in 51 (81%) patients and were mostly grade 1 or 2. Note that 3 patients (10.3%) had grade 3 maculopapular rashes and 31% of patients experienced nail disorders (includes nail disorder, nail ridging, onychomadesis, onychomadesis, and nail dystrophy). Three (4.8%) patients had ICANS events (grade ≥3: 1 (1.6%)) and all events resolved. The grade 3 ICANS event in one patient led to the talquetamab-related treatment discontinuation. A summary of the adverse events (AEs) for patients treated with talquetamab and daratumumab is shown in Table 14.

TABLE 14

Adverse events (AEs)

| Most common AEs (≥20%), n (%) | Tal + Dara^{a} (N=63) | |
|---|---|---|
| | Any Grade | Grade ¾ |
| Hematologic | | |
| Anemia | 29 (46) | 14 (22.2) |
| Thrombocytopenia | 23 (36.5) | 13 (20.6) |
| Neutropenia | 22 (34.9) | 16 (25.4) |
| Lymphopenia | 17 (27.0) | 17 (27.0) |
| Nonhematologic | | |
| CRS | 45 (71.4) | 0 (0) |
| Dysgeusia | 37 (58.7) | NA |
| Dry mouth | 28 (44.4) | 0 (0) (G4 NA) |
| Skin exfoliation | 24 (38.1) | 0 (0) |
| Fatigue | 20 (31.7) | 1 (1.6) |
| Pyrexia | 16 (25.4) | 1 (1.6) |
| Headache | 15 (23.8) | 0 (0) |
| Nausea | 14 (22.2) | 0 (0) |
| Weight decreased | 14 (22.2) | 0 (0) |
| Back pain | 14 (22.2) | 1 (1.6) |
| Decreased appetite | 13 (20.6) | 0 (0) |
| Arthralgia | 13 (20.6) | 0 (0) |
| Dizziness | 13 (20.6) | 0 (0) |
| Cough | 13 (20.6) | 0 (0) |
| Oropharyngeal pain | 13 (20.6) | 0 (0) |

^{a}Dara 1800 mg plus Tal (400 µg/kg SC QW or 400 µg/kg SC Q2W or 800 µg/kg SC Q2W) AE, adverse event; CRS, cytokine release syndrome; Dara, daratumumab; ICANS, immune effector cell-associated neurotoxicity syndrome; N/A, not applicable; PD, progressive disease; Q2W. every other week; QW, weekly; SC, subcutaneous; SOC, system organ class; Tal, talquetamab.

Among all daratumumab plus talquetamab cohorts (1800 mg daratumumab plus talquetamab (400 µg/kg SC QW or 400 µg/kg SC Q2W or 800 µg/kg SC Q2W)), there were no grade 3 or 4 Cytokine Release Syndrome (CRS) events. All CRS events were limited to grade 1 and 2 and were mostly confined to step-up and full doses of talquetamab. All CRS events resolved. Table 15 summarizes the CRS events.

TABLE 15

Cytokine Release Syndrome (CRS) events for all daratumumab plus talquetamab cohorts

| Parameter | Tal + Dara^{a} (n=63) |
|---|---|
| Patients with CRS, n (%) | 45 (71.4) |
| Time to onset, days^{b}, median (range) | 2 (1-4) |
| Duration, days, median (range) | 2 (1-28) |
| Supportive measures^{c}, n (%) | 40 (63.5) |
| Tocilizumab^{d} | 20 (31.7) |
| Steroids | 2 (3.2) |
| Oxy gen | 3 (4.8) |

^{a}Dara 1800 mg plus Tal (400 µg/kg QW + 400 µg/kg and 800 µg/kg Q2W);
^{b}Relative to the most recent dose;
^{c}A patient could receive > 1 supportive therapy;
^{d}Tocilizumab was allowed for all CRS events Q2W, every other week; QW, weekly.

In the daratumumab plus talquetamab 400 µg/kg weekly cohort, six (42.9%) participants discontinued talquetamab treatment; four (28.6%) participants due to progressive disease and 2 (14.3%) participant refused further study treatment. Four of the 5 participants in the daratumumab plus talquetamab 400 µg/kg biweekly cohort discontinued treatment due to death (n= 2 [40%]), TEAE (n=1 [20%]) (treatment-emergent adverse events) and physician decision (n=1 [20%]). In the daratumumab plus talquetamab 800 µg/kg biweekly cohort, five (11.4%) participants discontinued talquetamab treatment due to progressive disease, one (2.3%) each due to death, TEAE, and physician decision.

Participants in each cohort of daratumumab plus 400 µg/kg weekly (n=10 [71.4%]), 800 µg/kg biweekly (n=34 [77.3%]) SC talquetamab and 400 µg/kg biweekly (n=1 [20%]) experienced events of treatment-emergent CRS, all of which were non-serious, Grade 1-2 events and resolved. No participants experienced Grade 3 or 4 treatment-emergent symptoms of CRS. All participants who received SC talquetamab, except one in the daratumumab plus talquetamab 400 µg/kg weekly cohort, experienced ≥1 TEAE.

TEAEs were reported in 14 (100%), 5 (100%) and 43 (97.7%) of participants in the daratumumab plus talquetamab 400 µg/kg weekly, 400 µg/kg biweekly and 800 µg/kg biweekly cohorts, respectively. The highest proportions of TEAEs were dysgeusia, lymphopenia, neutropenia, thrombocytopenia, nail disorder, rash (3 participants had Grade ≥3), skin exfoliation, and CRS. Most common all-grade hematologic AEs were anemia (46% at any grade, 22.2% at grade 3 or 4), thrombocytopenia (36.5% at any grade, 20.6% at grade 3 or 4), and neutropenia (34.9% at any grade, 25.4% at grade 3 or 4). Most common all-grade nonhematologic AEs were CRS (71.4% at any grade, 0 at grade 3 or 4), dysgeusia (58.7% at any grade, grade 3 or 4 not applicable), and dry mouth (44.4% at any grade, 0 at grade 3, grade 4 not applicable). 34 (54%) participants experienced an infection-related TEAE; 19% participants had Grade ≥3 infection-related TEAE. The TEAEs of infection were pneumonia, Covid-19, and upper respiratory tract infection. TEAEs of injection-related reaction have been reported and all were Grade 1-2. One subject in the cohort treated biweekly with 800 µg/kg of talquetamab exhibited a DLT (dose-limiting toxicity).

Fifty-six participants across all talquetamab dose levels and daratumumab had ≥1 post dose disease evaluation as of 6 Apr. 2022 (i.e., were evaluable for efficacy). The responses included 7 subjects (12.5%) with a stringent complete response (sCR), 9 subjects (16.1%) with a complete response (CR), 20 subjects (35.7%) with a very good partial response (VGPR), and 9 subjects (16.1%) with a partial response (PR). Additionally, 8 subjects (14.3%) had stable disease and 3 subjects (5.4%) had progressive disease as best response. The overall response rate for patients treated with daratumumab and talquetamab was 80.4% (45 subjects). A summary of the overall response rate for patients treated with 400 and 800 µg/kg of talquetamab is shown in Table 10.

For subjects who received 1800 mg SC daratumumab and 400 µg/kg SC talquetamab weekly treatment dose (n=14 evaluable subjects), the responses included 2 subjects (14.3%) with a sCR, 2 subjects (14.3%) with a CR, 4 subjects (28.6%) with a VGPR, 2 subjects (14.3%) with a PR, and 4 subjects (28.6%) with stable disease. For subjects who received 1800 mg SC daratumumab and 400 µg/kg SC talquetamab biweekly treatment dose (n=5 evaluable subjects), the responses included 1 subject (20%) with a CR, 3 subjects (60%) with a VGPR, and 1 subject (20%) with progressive disease. For subjects who received 1800 mg SC daratumumab and 800 µg/kg SC talquetamab biweekly treatment dose (n=37 evaluable subjects), the responses included 5 (13.5%) sCR, 6 (16.2%) CR, 13 subjects (35.1%) with a VGPR, 7 subjects (18.9%) with a PR, and 4 subjects (28.6%) with stable disease. See Table 11 for additional information regarding patients treated with daratumumab and talquetamab (400 µg/kg weekly or 400 µg/kg and 800 µg/kg biweekly).

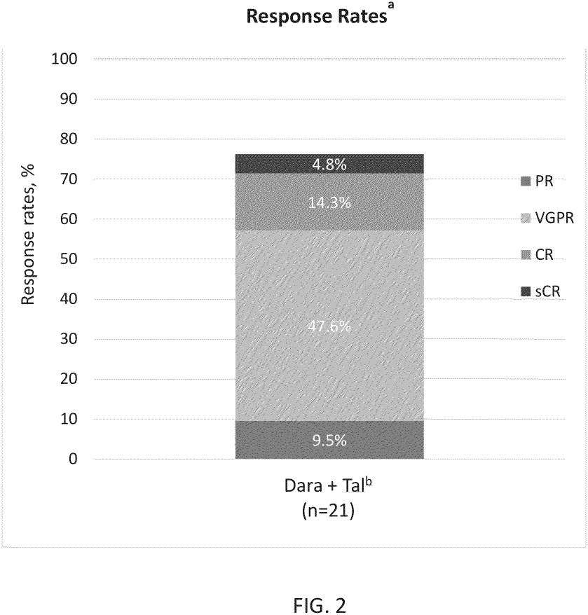

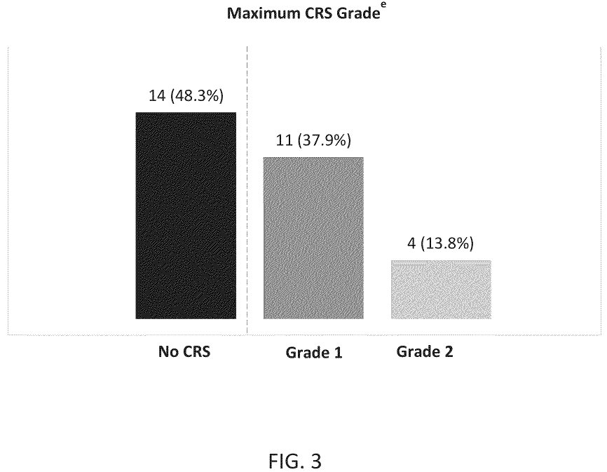

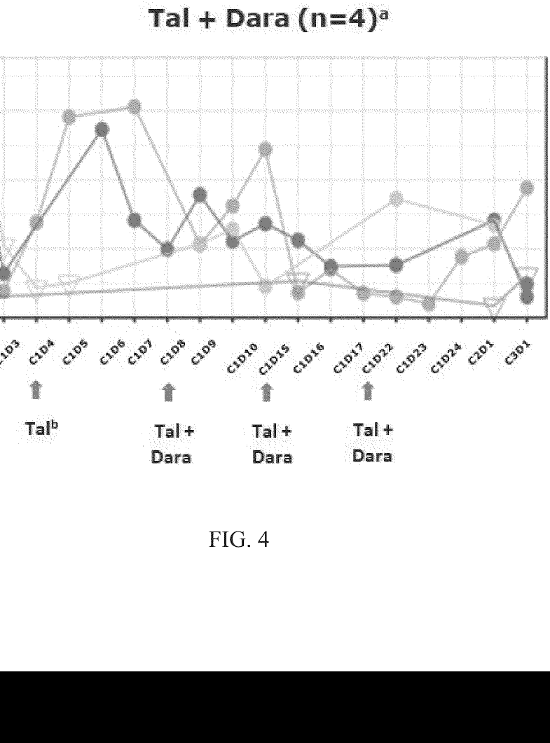

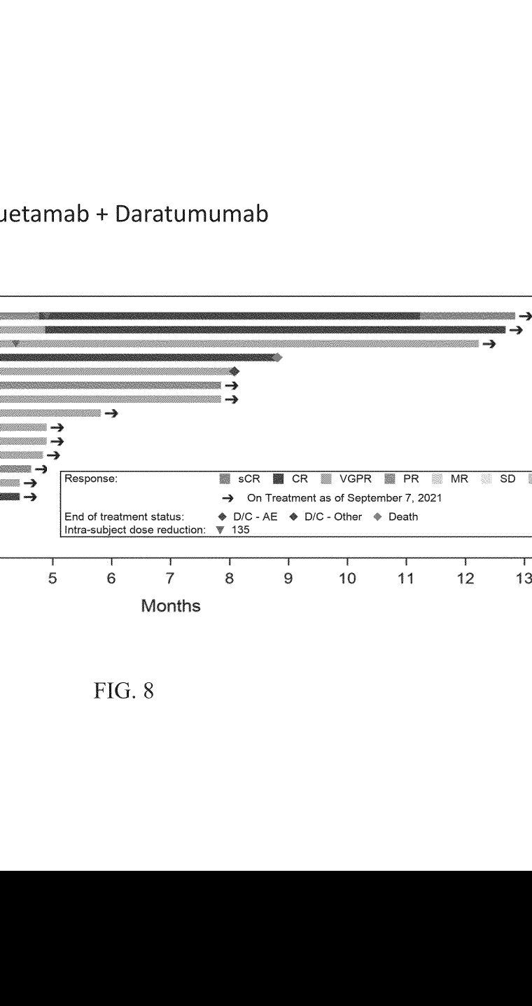

Consequently, treatment with daratumumab (1800 mg) and talquetamab (400 µg/kg weekly or 400 µg/kg or 800 µg/kg biweekly) exhibited preliminary efficacy in heavily pretreated patients (including patients with prior anti-CD38 treatment) with multiple myeloma. The median follow-up time was 5.59 months (range: 0.2-19.6). Responses occurred early (within 1 month) and continued to deepen over time. The response rates for patients who received ≥ 1 study treatment and had ≥1 post-baseline response evaluation is shown in FIG. 2. The median time to first confirmed response was 1 month (range: 0.9-6.5). The responses were durable and deepened over time (FIG. 8). No deaths occurred due to progressive disease (PD) in the talquetamab and daratumumab dosing cohorts. At a median follow-up of 6.5 months (range 1.6-19.6) for responders, 37/41 responders (90.2%) were continuing on treatment. Note that 31 out of 41 responders (75.6%) had prior anti-CD38 exposure.

Biomarker data indicated pharmacodynamic changes that were characteristic of the mechanism of action for talquetamab and daratumumab that were consistent to talquetamab monotherapy. These included T cell redistributions, as indicated by changes in absolute counts of CD3+ T cells, and increased expression of several T cell activation markers such as PD-1, LAG-3, TIM-3, HLA-DR, CD38, and CD25 on CD3+ T cells. In particular, the proportion of CD38+/CD8+ T cells declined after initial daratumumab dosing on C1D1, consistent with previous data with daratumumab, but notably, talquetamab administration led to induction of CD38+ CD8+T cells after the initial doses of talquetamab despite the concurrent daratumumab dosing. Increases in cytokines were also observed after the administration of talquetamab and daratumumab consistent with talquestamab monotherapy; these included IL-10, IL-6, and IL-2Rα. Additionally, the pharmacokinetic profile of talquetamab in the presence of daratumumab was consistent with the profile of observed in the phase 1 talquetamab monotherapy.

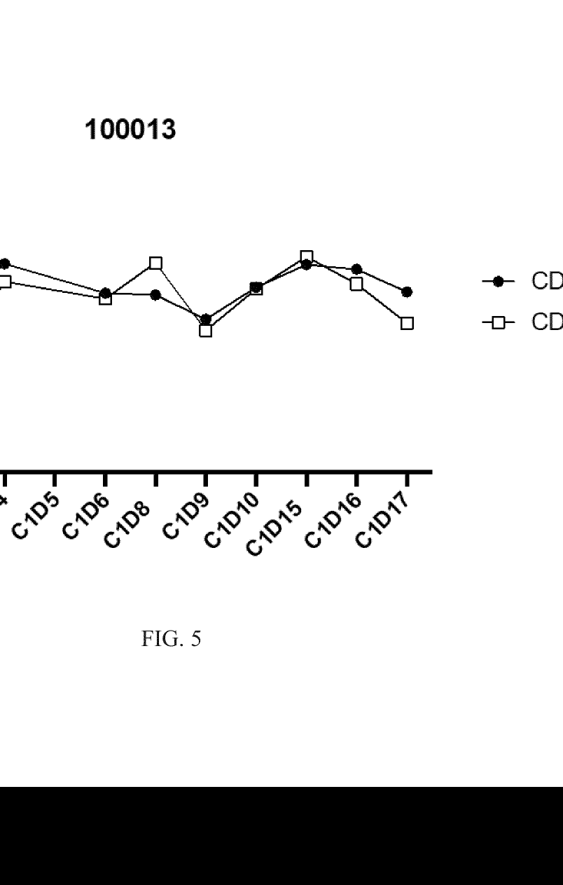

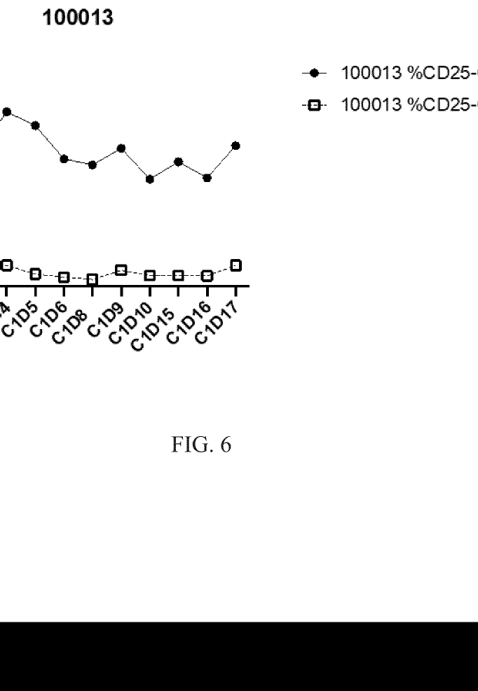

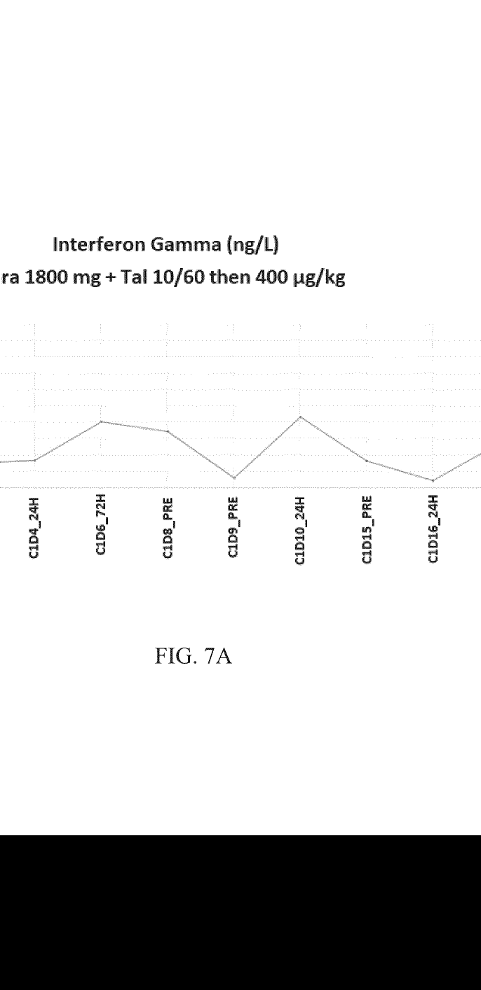

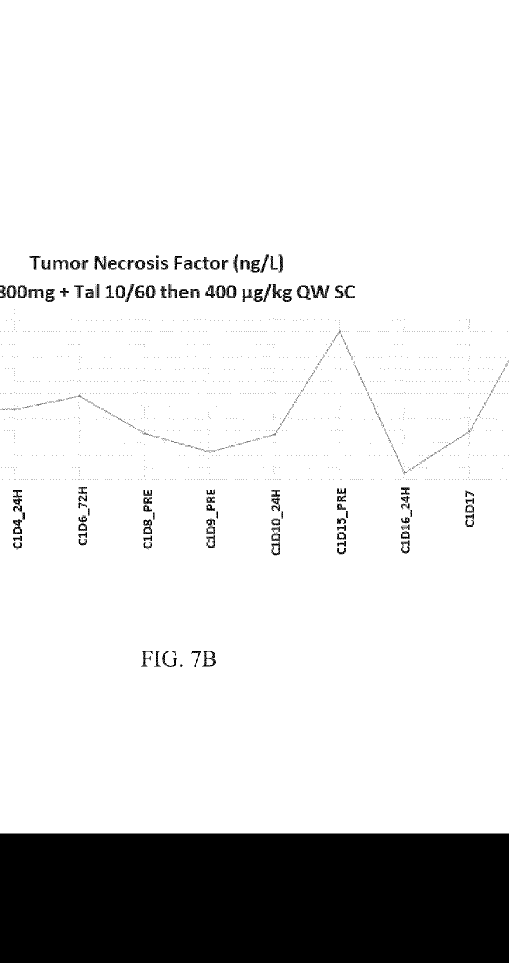

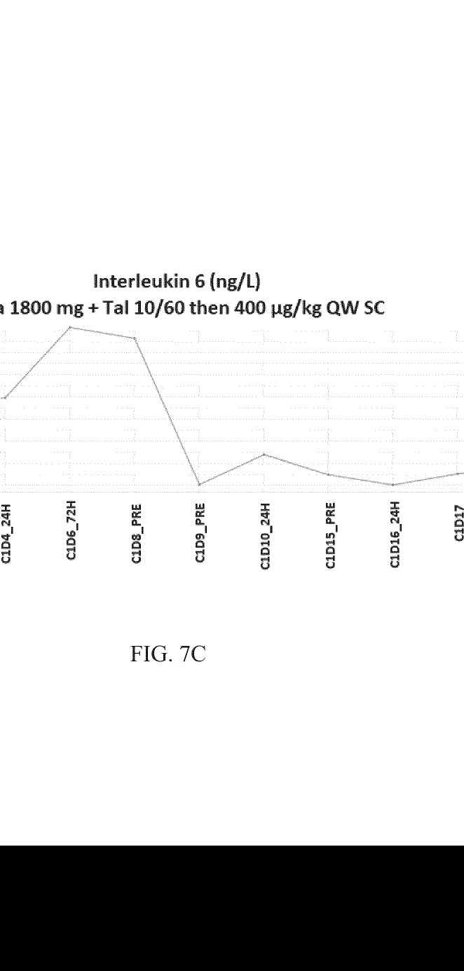

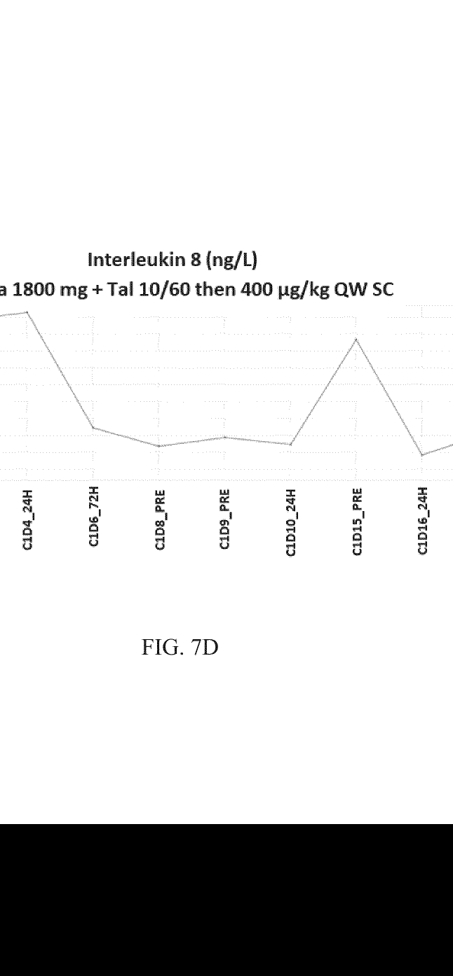

There was a transient reduction in T cell numbers early after talquetamab and daratumumab dosing, which was followed by recovery in T cell numbers within one week (FIG. 5). There was also an expansion in T cell numbers by Cycle 3 Day 1 (C3D1). Moreover, T cell activation was induced by talquetamab combination with daratumumab (FIG. 6). Induction of pro-inflammatory cytokines occurred following daratumumab and talquetamab treatment (FIGS. 7A-7D).

The preliminary data also included results for: 8 participants treated with 400 µg/kg SC talquetamab weekly, in combination with 1800 mg daratumumab SC and 4 mg pomalidomide (Tal400qwDaraPom4): 18 participants treated with 400 µg/kg SC talquetamab weekly, in combination with 1800 mg daratumumab SC and 2 mg pomalidomide (Tal400qwDaraPom2); and 60 participants treated with 800 µg/kg SC talquetamab biweekly, in combination with 1800 mg daratumumab SC and 2 mg pomalidomide (Tal800q2wDaraPom2). In the 400 µg/kg and 800 µg/kg biweekly cohorts pomalidomide was administered starting on Day 1 of Cycle 2. Dexamethasone was given as pretreatment medication during step up dosing and for the first treatment dose of talquetamab SC, as well as through Cycle 4. Participant demographics are detailed in Table 13. A detailed summary of the response rates for patients treated with talquetamab, daratumumab and pomalidomide is shown in Table 16.

The safety data is presented in 3 separate populations for talquetamab with pomalidomide subjects in combination with daratumumab 1800 mg: talquetamab 800 µg/kg and 2 mg pomalidomide starting at Cycle 2 Day 1 followed by an increase to 4 mg on C4D1 if certain criteria were met (Cohort 19, n=8), talquetamab 400 µg/kg weekly and pomalidomide 4 mg starting Cycle 1 Day 15 (Cohort 8, n=8) and pooled -talquetamab 400 µg/kg weekly or talquetamab 800 µg/kg every week with Pom 2 mg starting at Cycle 2 Day 1 (Cohorts 12, 13 and 18, n=50).

Of eight participants with talquetamab 800 µg/kg and pom starting at Cycle 2 Day 1, in combination with daratumumab 1800 mg, five (62.5%) participants reported serious adverse events. Five (62.5%) participants experienced CRS, all of which were Grade 1. Six participants (75%) had ≥1 TEAEs, most common events were lymphopenia (50%), headache (37.5%), skin exfoliation (25%) and dysgeusia (25%).

Of eight subjects in daratumumab 1800 mg with talquetamab 400 µg/kg weekly and Pom 4 mg starting Cycle 4 Day 1, one DLTs of thrombocytopenia was reported. Four (50%) participants reported serious adverse events. Five (62.5%) participants experienced CRS, all of which were Grade 1-2. No participants reported neurotoxicity related to talquetamab SC. Eight participants (100%) had ≥1 TEAEs, most common events were neutropenia (75%), dysgeusia (75%), pyrexia (75%), fatigue (62.5%), anemia (50%), diarrhea (50%), headache (50%), dry mouth (37.5%), decreased appetite (37.5%), pruritus (37.5%), nail disorder (37.5%), insomnia (37.5%) and Covid-19 (37.5%).

Of 50 participants in the pooled population of talquetamab 400 µg/kg weekly or talquetamab 800 µg/kg every week with Pom 2 mg in combination of daratumumab 1800 mg, one DLT of neutropenia was reported. 22 (44%) participants reported serious adverse events. 34 (68%) participants experienced CRS, all of which were Grade 1-2. 48 participants (96%) had ≥1 TEAEs. Most common events were dysgeusia (72%), neutropenia (64%), dry mouth (62%), skin exfoliation (44%), fatigue (42%), lymphopenia (36%), headache (34%), thrombocytopenia (30%) and anemia (30%).

TABLE 16

Summary of overall best response based on Investigator Assessment for indicated cohorts
TEFRESP02A1_ASH: Summary of Overall Best Response based on Investigator Assessment; Response Evaluable Subjects by Investigators (Study 64407564MMY1002)

| | talquetamab |
|---|---|
| | 400 µg/kg (SC3 and SC6) + 800 µg/kg (SC7 and SC15) |
| Analysis set: Response evaluable subjects by investigators | 56 |
| Response category | |
| Stringent complete response (sCR) | 7 (12.5%) |
| Unconfirmed | 0 |
| Complete response (CR) | 9 (16.1%) |
| Unconfirmed | 3 |
| Very good partial response (VGPR) | 20 (35.7%) |
| Unconfirmed | 5 |
| Partial response (PR) | 9 (16.1%) |
| Unconfirmed | 4 |
| Minimal response (MR) | 0 |
| Unconfirmed | - |
| Stable disease (SD) | 8 (14.3%) |
| Progressive disease (PD) | 3 (5.4%) |
| Not evaluable (NE) | 0 |

TEFRESP02A1_ASH: Summary of Overall Best Response based on Investigator Assessment; Response Evaluable Subjects by Investigators (Study 64407564MMY1002)

| | talquetamab |
|---|---|
| | 400 µg/kg (SC3 and SC6) + |
| | 800 µg/kg (SC7 and SC15) |
| Overall response (sCR+CR+VGPR+PR) | 45 (80.4%) |
| Clinical benefit (Overall response + MR) | 45 (80.4%) |
| VGPR or better (sCR + CR + VGPR) | 36 (64.3%) |
| CR or better (sCR + CR) | 16 (28.6%) |

Response evaluable subjects by investigators: Subjects have received at least one study treatment and have at least one post-baseline response evaluation by investigator.
Note: Response was assessed by investigators, based on IMWG Criteria. Confirmed response require at least two consecutive identical investigators’ response assessments.
Unconfirmed responders do not have two consecutive investigators’ response assessments to confirm the response at the time of this data review.
Percentages are calculated with the number of subjects in each group as denominator.
[TEFRESP02A1_ASH.RTF][JNJ-64407564\MMY1002\DBR_TAL_EOP2\RE_TAL_EOP2\PROD\TEFRESP02A1_ASH.SAS ] 20SEP2021, 15:36

TABLE 17

Summary of overall best response based on Investigator Assessment (response evaluable subjects by investigators)
TEFRESP02A1: Summary of Overall Best Response based on Investigator Assessment; Response Evaluable Subjects by Investigators (Study 64407564MMY1002)
talquetamab

| SC Weekly | | | | SC Bi-weekly | | | | |
|---|---|---|---|---|---|---|---|---|
| | Dara 1800 mg + Tal 400 µg/kg | Pom 4 mg del + Dara 1800 mg + Tal 400 µg/kg | Pom 2 mg + Dara 1800 mg + Tal 400 µg/kg C2D1 | Dara 1800 mg + Tal 400 µg/kg C2D1 | Dara 1800 mg + Tal 800 µg/kg C1D15 | Pom 2 mg + Dara 1800 mg + Tal 800 µg/kg C2D1 | Tal Total | Overall Total |
| Analysis set: Response evaluable subjects by investigators | 14 | 8 | 18 | 5 | 37 | 26 | 108 | 186 |
| Response category Stringent complete response (sCR) | 2 (14.3%) | 1 (12.5%) | 3 (16.7%) | 0 | 5 (13.5%) | 3 (11.5%) | 14 (13.0%) | 18 (9.7%) |
| Complete response (CR) | 2 (14.3%) | 2 (25.0%) | 0 | 1 (20.0%) | 6 (16.2%) | 3 (11.5%) | 14 (13.0%) | 28 (15.1%) |
| Very good partial response (VGPR) | 4 (28.6%) | 4 (50.0%) | 8 (44.4%) | 3 (60.0%) | 13 (35.1%) | 11 (42.3%) | 43 (39.8%) | 78 (41.9%) |
| Unconfirmed | 2 | 1 | 2 | 0 | 2 | 2 | 13 | 11 |
| Partial response (PR) | 2 (14.3%) | 1 (12.5%) | 7 (27.8%) | 0 | 7 (18.9%) | 3 (11.5%) | 18 (16.7%) | 22 (11.8%) |
| Minimal response (MR) | 0 | 0 | 0 | 0 | 0 | 0 | 0 | 0 |
| Unconfirmed | - | - | - | - | - | - | - | - |
| Stable disease (SD) | 4 (28.6%) | 0 | 2 (11.1%) | 0 | 4 (10.8%) | 5 (19.2%) | 15 (13.9%) | 29 (15.6%) |
| Progressive disease (PD) | 0 | 0 | 0 | 1 (20.0%) | 2 (5.4%) | 1 (25.0%) | 4 (3.7%) | 11 (5.9%) |

TEFRESP02A1: Summary of Overall Best Response based on Investigator Assessment; Response Evaluable Subjects by Investigators (Study 64407564MMY1002)
talquetamab

| | SC Weekly | | | SC Bi-weekly | | | | |
|---|---|---|---|---|---|---|---|---|
| | Dara 1800 mg + Tal 400 µg/kg | Pom 4 mg del + Dara 1800 mg + Tal 400 µg/kg | Pom 2 mg + Dara 1800 mg + Tal 400 µg/kg C2D1 | Dara 1800 mg + Tal 400 µg/kg C2D1 | Dara 1800 mg + Tal 800 µg/kg C1D15 | Pom 2 mg + Dara 1800 mg + Tal 800 µg/kg C2D1 | Tal Total | Overall Total |
| Not evaluable (NE) | 0 | 0 | 0 | 0 | 0 | 0 | 0 | 1 (1.2%) |
| Overall response (sCR+CR+VGPR+P R) | 10 (71.4%) | 8 (100%) | 16 (88.9%) | 4 (80.0%) | 31 (83.8%) | 20 (76.9%) | 89 (82.4%) | 146 (78.5%) |
| Clinical benefit (Overall response + MR) | 10 (71.4%) | 8 (100%) | 16 (88.9%) | 4 (80.0%) | 31 (83.8%) | 20 (76.9%) | 89 (82.4%) | 146 (78.5%) |
| VGPR or better (sCR + CR + VGPR) | 8 (57.1%) | 7 (87.5%) | 11 (61.1%) | 3 (60.0%) | 24 (64.9%) | 17 (65.4%) | 71 (65.7%) | 124 (66.7%) |
| CR or better (sCR + CR) | 4 (28.6%) | 3 (37.5%) | 3 (16.7%) | 1 (20.0%) | 11 (29.7%) | 6 (23.1%) | 28 (25.9%) | 46 (24.7%) |

TEFRESP02A1: Summary of Overall Best Response based on Investigator Assessment; Response Evaluable Subjects by Investigators (Study 64407564MMY1002)
talquetamab

| | | | | | | | |
|---|---|---|---|---|---|---|---|
| SC Weekly | | | SC Bi-weekly | | | | |
| Dara 1800 mg + Tal 400 µg/kg | Pom 4 mg del + Dara 1800 mg + Tal 400 µg/kg | Pom 2 mg + Dara 1800 mg + Tal 400 µg/kg C2D1 | Dara 1800 mg + Tal 400 µg/kg C2D1 | Dara 1800 mg + Tal 800 µg/kg C1D15 | Pom 2 mg + Dara 1800 mg + Tal 800 µg/kg C2D1 | Tal Total | Overall Total |

Response evaluable subjects by investigators: Subjects have received at least one study treatment and have at least one post- baseline response evaluation by investigator.
Note: Response was assessed by investigators, based on IMWG Criteria. Confirmed response require at least two consecutive identical investigators’ response assessments.
Unconfirmed responders do not have two consecutive investigators’ response assessments to confirm the response at the time of this data review. Percentages are calculated with the number of subjects in each group as denominator.
[TEFRESP02A1.RTF] [JNJ-64407564\MMY1002\DBR_TAL_EOP2\RE_TAL_EOP2\PROD\TEFRESP02A1.SAS] 20SEP2021, 15:36

Forty-four participants across all talquetamab, daratumumab and pomalidomide dose levels had ≥1 post dose disease evaluation as of Apr. 6, 2022 (i.e., were evaluable for efficacy). The responses included 6 subjects (13.6%) with a stringent complete response (sCR), 3 subjects (6.8%) with a CR, 19 subjects (43.2%) with a very good partial response (VGPR), and 8 subjects (18.2%) with a partial response (PR). Additionally, 7 subjects (15.9%) had stable disease and 1 subject (2.3%) had progressive disease as best response. See also FIG. 2.

For subjects who received 1800 mg SC daratumumab, 400 µg/kg SC talquetamab weekly and 4 mg pomalidomide treatment dose (n=8 evaluable subjects), the responses included 1 subject (12.5%) with a sCR, 2 subjects (25%) with a CR, 4 subjects (50%) with a VGPR, and 1 subject (12.5%) with partial response. For subjects who received 1800 mg SC daratumumab, 400 µg/kg SC talquetamab weekly and 2 mg pomalidomide treatment dose (n=18 evaluable subjects), the responses included 3 subjects (16.7%) with a sCR, 8 subject (44.4%) with VGPR, 5 subjects (27.8%) with a PR, and 2 subjects (11.1%) with stable. For subjects who received 1800 mg SC daratumumab, 800 µg/kg SC talquetamab biweekly and 2 mg pomalidomide treatment dose (n=26 evaluable subjects), the responses included 3 subjects (11.5%) with sCR, 3 subjects (11.5%) with CR, 11 subject (542.3%) with a VGPR, 3 subjects (11.5%) with PR, 5 subjects (19.2%) with stable disease and 1 subject (3.8%) with progressive disease. No efficacy data was available for the cohort of eight subjects with talquetamab 400 µg/kg weekly and pomalidomide, 2 mg and then 4 mg starting Cycle 4 Day 1, as of Apr. 6, 2022.

Overall, the data was promising and warranted further investigation. Consequently, a phase 3 study will be conducted that examines administration of talquetamab, daratumumab SC, and pomalidomide (Tal-Dara-Pom) vs. talquetamab and daratumumab SC (Tal-Dara) vs. daratumumab SC, pomalidomide, and dexamethasone (DPd) in patients with RRMM.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

What is claimed:

1. A method of treating a cancer in a subject in need thereof, comprising:
(1) administering to the subject a GPRC5DxCD3 bispecific antibody at a dose of 60 µg/kg to 1200 µg/kg every 1-2 weeks; and
(2) administering to the subject an anti-CD38 antibody at a dose of 1200 mg to 2400 mg every 1-4 weeks.

2. The method of claim 1, wherein the method comprises:
(1) subcutaneously administering to the subject the GPRC5DxCD3 bispecific antibody at a dose of 300 µg/kg to 1200 µg/kg every 1-2 weeks; and
(2) subcutaneously administering to the subject the anti-CD38 antibody at a dose of 1600 mg to 2000 mg every 1-4 weeks.

3. The method of claim 2, further comprising subcutaneously administering to the subject the GPRC5DxCD3 bispecific antibody at a dose lower than that used in step (1) prior to step (1).

4. The method of claim 2, wherein the GPRC5DxCD3 bispecific antibody is subcutaneously administered to the subject at a dose of about 300 µg/kg, 400 µg/kg, 450 µg/kg, 500 µg/kg, 550 µg/kg, 600 µg/kg, 700 µg/kg, 750 µg/kg, 800 µg/kg, 850 µg/kg, 900 µg/kg, 950 µg/kg, or 1000 µg/kg, or any dose from about 300 µg/kg to about 1000 µg/kg, once every week or once every two weeks.

5. The method of claim 4, wherein the GPRC5DxCD3 bispecific antibody is subcutaneously administered to the subject at a dose of 400 µg/kg weekly or biweekly, or 800 µg/kg biweekly.

6. The method of claim 5, wherein the GPRC5DxCD3 bispecific antibody is subcutaneously administered to the subject at a dose of 400 µg/kg weekly or 800 µg/kg biweekly.

7. The method of claim 1, wherein the anti-CD38 antibody is subcutaneously administered to the subject at the dose of 1800 mg once every week during week 1 to week 8 of the treatment, once every two weeks during week 9 to week 24 of the treatment, and once every four weeks after week 24 of the treatment.

8. The method of claim 1, wherein the anti-CD38 antibody is administered or provided for administration together with rHuPH20 .

9. The method of claim 1, wherein the GPRC5DxCD3 bispecific antibody comprises:
(1) a GPRC5D binding domain comprising a heavy chain variable region (VH) comprising heavy chain complementarity determining regions (HCDRs) HCDR1, HCDR2 and HCDR3 of the amino acid sequences of SEQ ID NO: 27, SEQ ID NO: 28, and SEQ ID NO: 29, respectively, and a light chain variable region (VL) comprising light chain complementarity determining regions (LCDRs) LCDR1, LCDR2 and LCDR3 of the amino acid sequences of SEQ ID NO: 30, SEQ ID NO: 31, and SEQ ID NO: 32, respectively, and
(2) a CD3 binding domain comprising a VH comprising HCDR1, HCDR2 and HCDR3 of the amino acid sequences of SEQ ID NO: 17, SEQ ID NO: 18, and SEQ ID NO: 19, respectively, and a VL comprising LCDR1, LCDR2 and LCDR3 of the amino acid sequences of SEQ ID NO: 20, SEQ ID NO: 21, and SEQ ID NO:22, respectively.

10. The method of claim 9, wherein the GPRC5D binding domain comprises the VH comprising the amino acid sequence of SEQ ID NO: 33 and the VL comprising the amino acid sequence of SEQ ID NO: 34; the CD3 binding domain comprises the VH comprising the amino acid sequence of SEQ ID NO: 23 and the VL comprising the amino acid sequence of SEQ ID NO: 24.

11. The method of claim 10, wherein the GPRC5DxCD3 bispecific antibody comprises a first heavy chain (HC1) comprising the amino acid sequence of SEQ ID NO: 35, a first light chain (LC1) comprising the amino acid sequence of SEQ ID NO: 36, a second heavy chain (HC2) comprising the amino acid sequence of SEQ ID NO: 25, and a second light chain (LC2) comprising the amino acid sequence of SEQ ID NO: 26.

12. The method of claim 1, wherein the anti-CD38 antibody comprises a VH comprising HCDR1, HCDR2 and HCDR3 of the amino acid sequences of SEQ ID NO: 7, SEQ ID NO: 8, and SEQ ID NO: 9, respectively, and a VL comprising LCDR1, LCDR2 and LCDR3 of the amino acid sequences of SEQ ID NO: 10, SEQ ID NO: 11, and SEQ ID NO: 12, respectively.

13. The method of claim 12, wherein the anti-CD38 antibody comprises the VH comprising the amino acid sequence of SEQ ID NO: 5, and the VL comprising the amino acid sequence of SEQ ID NO: 6.

14. The method of claim 1, wherein the cancer is multiple myeloma.

15. A method of treating multiple myeloma in a subject in need thereof, comprising:
(1) subcutaneously administering to the subject 400 µg/kg of a GPRC5DxCD3 bispecific antibody weekly; or 800 µg/kg of a GPRC5DxCD3 bispecific antibody biweekly, and
(2) subcutaneously administering to the subject 1800 mg of an anti-CD38 antibody once every week during week 1 to week 8 of the treatment, once every two weeks during week 9 to week 24 of the treatment, and once every four weeks after week 24 of the treatment, wherein the GPRC5DxCD3 bispecific antibody comprises a first heavy chain (HC1) of SEQ ID NO: 35, a first light chain (LC1) of SEQ ID NO: 36, a second heavy chain (HC2) of SEQ ID NO: 25, and a second light chain (LC2) of SEQ ID NO: 26, and the anti-CD38 antibody comprises the HC of SEQ ID NO: 13 and the LC of SEQ ID NO: 14.

16. The method of claim 15, further comprising subcutaneously administering to the subject one or more first step-up doses of the GPRC5DxCD3 bispecific antibody, wherein said first step up doses comprise 10 µg/kg on day 2 of the treatment and 60 µg/kg on day 4 of the treatment, prior to the initial dose of 400 µg/kg of the GPRC5DxCD3 bispecific antibody, or subcutaneously administering to the subject one or more second step-up doses of the GPRC5DxCD3 bispecific antibody, wherein said second step up doses comprise 10 µg/kg on day 2 of the treatment, 60 µg/kg on day 4 of the treatment, and 300 µg/kg on day 8 of the treatment, prior to the initial dose of 800 µg/kg of the GPRC5DxCD3 bispecific antibody.

17. The method of claim 1, wherein the subject has received at least one prior treatment for multiple myeloma, or the subject is relapsed or refractory to the at least one prior treatment, wherein the prior treatment comprises at least one of a proteasome inhibitor (PI) or an immunomodulatory agent (IMiD).

18. The method of claim 17, wherein the subject is refractory or relapsed to a treatment selected from the group consisting of an anti-CD38 antibody, lenalidomide, bortezomib, pomalidomide, carfilzomib, elotuzumab, ixazomib, isatuximab, melphalan and thalidomide, or any combination thereof.

19. The method of claim 1, further comprising administering to the subject an additional therapeutic, wherein the additional therapeutic is pomalidomide and/or dexamethasone.

20. The method of claim 1, wherein said treating results in T-cell activation, wherein said T-cell activation comprises an increase in at least one of CD25, PD-1, CD38 on CD4 and CD8 T cells, or said treating results in an increase in frequency of at least of CD38+ CD8+ T cells, CD38+ CD4+ T cells and Tregs T cells.


SEQUENCE LISTING (69 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 3 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 4 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 5 - protein, 122 aa, Homo sapiens [sequence omitted]

SEQ ID NO: 6 - protein, 107 aa, Homo sapiens [sequence omitted]

SEQ ID NO: 7 - protein, 5 aa, Homo sapiens
SFAMS

SEQ ID NO: 8 - protein, 17 aa, Homo sapiens
AISGSGGGTYYADSVKG

SEQ ID NO: 9 - protein, 13 aa, Homo sapiens
DKILWFGEPVFDY

SEQ ID NO: 10 - protein, 11 aa, Homo sapiens
RASQSVSSYLA

SEQ ID NO: 11 - protein, 7 aa, Homo sapiens
DASNRAT

SEQ ID NO: 12 - protein, 9 aa, Homo sapiens
QQRSNWPPT

SEQ ID NO: 13 - protein, 452 aa, Homo sapiens
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 330 aa, Homo sapiens [sequence omitted]

SEQ ID NO: 16 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 5 aa, Homo sapiens
TYAMN

SEQ ID NO: 18 - protein, 19 aa, Homo sapiens
RIRSKYNNYATYYAASVKG

SEQ ID NO: 19 - protein, 14 aa, Homo sapiens
HGNFGNSYVSWFAY

SEQ ID NO: 20 - protein, 14 aa, Homo sapiens
RSSTGAVTTSNYAN

SEQ ID NO: 21 - protein, 7 aa, Homo sapiens
GTNKRAP

SEQ ID NO: 22 - protein, 9 aa, Homo sapiens
ALWYSNLWV

SEQ ID NO: 23 - protein, 125 aa, Homo sapiens
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 24 - protein, 112 aa, Homo sapiens
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 25 - protein, 452 aa, Homo sapiens
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 26 - protein, 215 aa, Homo sapiens
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - protein, 5 aa, Homo sapiens
GYTMN

SEQ ID NO: 28 - protein, 17 aa, Homo sapiens
LINPYNSDTNYAQKLQG

SEQ ID NO: 29 - protein, 9 aa, Homo sapiens
VALRVALDY

SEQ ID NO: 30 - protein, 11 aa, Homo sapiens
KASQNVATHVG

SEQ ID NO: 31 - protein, 7 aa, Homo sapiens
SASYRYS

SEQ ID NO: 32 - protein, 9 aa, Homo sapiens
QQYNRYPYT

SEQ ID NO: 33 - protein, 118 aa, Homo sapiens
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 34 - protein, 47 aa, Homo sapiens
RFSGSGSGTEFTLTISNLQPEDFATYYCQQYNRYPYTFGQGTKLEIK

SEQ ID NO: 35 - protein, 445 aa, Homo sapiens
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 210 aa, Homo sapiens
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 37 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 38 - protein, 122 aa, Homo sapiens
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 39 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 40 - protein, 122 aa, Homo sapiens
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 41 - protein, 107 aa, Homo sapiens
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 42 - protein, 120 aa, Homo sapiens
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 43 - protein, 109 aa, Homo sapiens
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 44 - protein, 120 aa, Homo sapiens [sequence omitted]

SEQ ID NO: 45 - protein, 107 aa, Homo sapiens [sequence omitted]

SEQ ID NO: 46 - protein, 7 aa, Homo sapiens
GFTFNSF

SEQ ID NO: 47 - protein, 6 aa, Homo sapiens
SGSGGG

SEQ ID NO: 48 - protein, 10 aa, Homo sapiens
GFTFNSFAMS

SEQ ID NO: 49 - protein, 10 aa, Homo sapiens
AISGSGGGTY

SEQ ID NO: 50 - protein, 8 aa, Homo sapiens
GFTFNSFA

SEQ ID NO: 51 - protein, 8 aa, Homo sapiens
ISGSGGGT

SEQ ID NO: 52 - protein, 15 aa, Homo sapiens
AKDKILWFGEPVFDY

SEQ ID NO: 53 - protein, 6 aa, Homo sapiens
QSVSSY

SEQ ID NO: 54 - protein, 7 aa, Homo sapiens
GYSFTGY

SEQ ID NO: 55 - protein, 6 aa, Homo sapiens
NPYNSD

SEQ ID NO: 56 - protein, 7 aa, Homo sapiens
GFTFNTY

SEQ ID NO: 57 - protein, 8 aa, Homo sapiens
RSKYNNYA

SEQ ID NO: 58 - protein, 10 aa, Homo sapiens
GYSFTGYTMN

SEQ ID NO: 59 - protein, 10 aa, Homo sapiens
LINPYNSDTN

SEQ ID NO: 60 - protein, 10 aa, Homo sapiens
GFTFNTYAMN

SEQ ID NO: 61 - protein, 12 aa, Homo sapiens
RIRSKYNNYATY

SEQ ID NO: 62 - protein, 8 aa, Homo sapiens
GYSFTGYT

SEQ ID NO: 63 - protein, 8 aa, Homo sapiens
INPYNSDT

SEQ ID NO: 64 - protein, 11 aa, Homo sapiens
ARVALRVALDY

SEQ ID NO: 65 - protein, 6 aa, Homo sapiens
QNVATH

SEQ ID NO: 66 - protein, 8 aa, Homo sapiens
GFTFNTYA

SEQ ID NO: 67 - protein, 10 aa, Homo sapiens
IRSKYNNYAT

SEQ ID NO: 68 - protein, 16 aa, Homo sapiens
ARHGNFGNSYVSWFAY

SEQ ID NO: 69 - protein, 9 aa, Homo sapiens
TGAVTTSNY